(19) United States
(12) Patent Application Publication
(10) Pub. No.: US 2025/0161269 A1
(43) Pub. Date: May 22, 2025

(54) COMPOSITIONS AND METHODS FOR PERIPHERAL TARGETING OF MELATONIN RECEPTOR AGONIST

(71) Applicant: Celmatix Inc., New York, NY (US)

(72) Inventors: Caterina CLEMENTI, New York, NY (US); Piraye Yurttas BEIM, New York, NY (US); Stephen S. PALMER, Groton, MA (US); Karen Hunter COHN, New York, NY (US)

(21) Appl. No.: 19/033,093

(22) Filed: Jan. 21, 2025

Related U.S. Application Data
(63) Continuation of application No. PCT/US2023/070722, filed on Jul. 21, 2023.
(60) Provisional application No. 63/391,622, filed on Jul. 22, 2022.

(51) Int. Cl. A61K 31/4045; A61K 31/165; A61K 31/343; A61K 31/4184

(52) U.S. Cl. CPC A61K 31/4045; A61K 31/165; A61K 31/343; A61K 31/4184

(57) ABSTRACT

Described herein are pharmaceutical compositions for melatonin receptor agonists for use in tissues outside of the CNS. Also described herein are methods for using the pharmaceutical compositions described herein for improving menopausal systems and for modulating the symptoms of and improving reproductive outcomes in polycystic ovary syndrome, endometriosis, chemotherapy-induced ovarian dysfunction, and inflammatory and metabolic conditions.

### CROSS-REFERENCE

This application is a continuation of International Application No. PCT/US2023/070722, filed Jul. 21, 2023, which claims the benefit of U.S. Provisional Patent Application No. 63/391,622, filed on Jul. 22, 2022, which are incorporated by reference herein in their entireties.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### TECHNICAL FIELD

The present disclosure relates to melatonin receptor agonist pharmaceutical compositions targeted to peripheral organs or peripheral tissues and/or melatonin receptor agonist pharmaceutical compositions that have reduced accumulation within the central nervous system following administration. The present disclosure also relates to methods for use of these melatonin receptor agonist pharmaceutical compositions for the prevention and/or treatment of symptoms relating to polycystic ovary syndrome (PCOS), endometriosis, menopause, inflammation of a reproductive tract, or chemotherapy-induced ovarian dysfunction (CIOD).

### BACKGROUND

Melatonin, N-acetyl-5-methoxytryptamine, functions as a pleiotropic hormone in numerous animal species. In vertebrates, a main source of melatonin production is the pineal gland which releases melatonin with a circadian production cycle that increases in the evenings, peaks in the middle of the night and then quickly drops until the morning hours and remains low during the daytime. Melatonin is a fast-acting hormone that is rapidly metabolized and maintains a short half-life inside the body (20-40 minutes). As such, serum concentrations of melatonin at any point in time track closely to pineal production levels. Melatonin is known to function to synchronize circadian rhythms and increasing concentrations in serum operate in the central nervous system (CNS) to induce somnolence and promote sleep in diurnal vertebrate species.

In addition to its hormonal functions, melatonin exhibits robust antioxidant properties. Melatonin and its metabolic derivatives possess strong free radical scavenging characteristics against both reactive oxygen species and reactive nitrogen species. By operating to directly scavenge these free radicals, melatonin and its derivatives can protect against cellular-, tissue-, and organ system-oxidative stress.

Melatonin also functions as a high affinity ligand for Melatonin receptor 1 A (MTNR1A, MT₁) and Melatonin receptor 1 B (MTNR1B, MT₂). MTNR1A and MTNR1B are G-protein coupled, 7-transmembrane receptors and are responsible for initiating melatonin-induced signal transduction within target cells throughout the body. Melatonin-induced signal transduction mediated through MTNR1A and MTNR1B is known to regulate mammalian circadian rhythms. In addition to its direct effect as an antioxidant, melatonin elicits an indirect increase in antioxidant mechanisms in target cells. Melatonin signaling through MTNR1A and MTNR1B induces the expression of antioxidant enzymes such as superoxide dismutase (SOD), glutathione reductase (GSR), glutathione peroxidase (GPX), and catalase (CAT).

### SUMMARY

There remains a need for modulation of a phenotype that is affected by melatonin in a subject. Melatonin receptors are expressed in the human ovary and melatonin directly affects ovarian function, without being mediated through the hypothalamus-pituitary-gonad (HPG) axis. In the ovary, melatonin functions to regulate ovarian steroidogenesis and to offset the peripheral (endocrine) and local (paracrine and intracrine) influences of inflammation and oxidative stress on ovarian somatic cells and maturation of the oocyte. Melatonin signaling through its receptors MT₁ and MT₂ regulates ovarian steroidogenesis, inflammation, oxidative stress and oocyte maturation in in vitro human cell studies.

In some instances, delivery of melatonin or a melatonin receptor agonist produces a beneficial modulation of a phenotype in a subject. Yet, a therapeutically effective amount of melatonin or a melatonin receptor agonist distributed systemically may cross the blood-brain barrier and elicit an unwanted neurological phenotype in the subject. As such, delivery of a therapeutically effective amount of melatonin or a melatonin receptor to target tissue outside the CNS may produce a beneficial modulation of the phenotype in the subject without unintentionally affecting the body functions mediated by the CNS or inducing unwanted side effects.

Melatonin, due to its amphiphilic characteristics (high hydrophilicity and lipophilicity), readily diffuses away from sources of production and can easily cross morphophysiological barriers including the blood-brain barrier and cell membranes. Melatonin may be distributed to all tissues via circulation. Extracellular melatonin equilibrates rapidly with cytoplasmic levels due its cell-penetrating characteristics leading to a variety of functional effects such as cell signaling initiated through binding to high affinity cell surface receptors (MT₁ and MT₂) and intracellular interactions such as direct antioxidant effects within cells and within subcellular compartments. Melatonin has two functional groups that determine its specificity and amphiphilicity. These are the 5-methoxy group and the N-acetyl side chain. Despite characteristics of melatonin and its production that propagate widespread distribution and equilibration, local concentrations on melatonin do not necessarily reflect serum melatonin concentration. Once synthesized by the pineal gland, melatonin is released directly into the cerebrospinal fluid (CSF) and the peripheral circulation (where approximately 70% of melatonin remains bound to serum albumin). Melatonin concentrations in the third ventricle are much higher than in other ventricular locations due to direct secretion from the pineal recess. Brain regions not directly in contact with the third ventricle may receive melatonin through circulating CSF or through the blood supply in which serum melatonin crosses the blood-brain barrier into local CNS regions. The pineal gland is not the sole source of melatonin production in vertebrates. Extrapineal sources of melatonin production include: retina, cerebellum, Harderian gland, cochlea, skin, testis, ovary, bone marrow, thymus, placenta, liver, kidney, heart, enterochromaffin cells of the digestive tract, peripheral blood mononuclear cells, and mast cells. Apart from in the retina, the extrapineal sources of melatonin production do not appear to follow the same circadian production rhythms as in the pineal gland and also do not appear to contribute substantially to circulating melatonin levels. Extrapineally-produced melatonin is believed to function locally in cells types in close proximity to production. Regulated transport of melatonin may also influence local melatonin concentrations or CNS bioavailability through blood-brain barrier transport.

Described herein are pharmaceutical compositions relating to the delivery of melatonin receptor agonists to tissues throughout the body of a subject. In some aspects, a pharmaceutical composition comprises a melatonin receptor agonist, wherein the pharmaceutical composition is formulated for intravaginal or intrauterine administration. In some aspects, the intravaginal or intrauterine administration intensifies a local effect of a melatonin receptor agonist. In some aspects, the intensification of the local effect is relative to a CNS exposure to a melatonin receptor agonist achieved across the blood-brain barrier. In some aspects, a pharmaceutical composition comprises a melatonin receptor agonist, wherein the pharmaceutical composition is formulated for systemic administration with reduced accumulation in the CNS or reduced penetration through the blood brain barrier. In some aspects, a pharmaceutical composition comprises a melatonin receptor agonist, wherein the pharmaceutical composition is formulated for systemic administration with reduced accumulation in the CNS or reduced penetration through the blood brain barrier as compared to an equivalent amount of melatonin. In some aspects, intensification of a local effect by a melatonin receptor agonist relative to a CNS exposure of a melatonin receptor agonist achieved across the blood-brain barrier occurs following local delivery of a melatonin receptor agonist, following delivery of a melatonin receptor agonist from a device, or following systemic delivery of a melatonin receptor agonist formulated for reduced accumulation in the CNS or formulated for reduction penetration through the blood-brain barrier. In some aspects, the pharmaceutical composition comprises a therapeutically effective amount of melatonin receptor agonist. In some aspects, the therapeutically effective amount is effective to treat or ameliorate symptoms of polycystic ovary syndrome (PCOS), endometriosis, menopausal symptoms (e.g. bone density loss, weight gain, hypertension, hair loss, inflammation), chemotherapy-induced ovarian dysfunction, or inflammation of reproductive tract in a subject in need thereof. In some embodiments, the melatonin receptor agonist comprises at least one of Circadin®, Slenyto®, Ramelteon, Tasimelteon, Agomelatine, TIK-301, Piromelatine, N-[2-(5-Chloro-2,6-dimethoxybenzoimidazol-1-yl)ethyl]acetamide (ACH000-143, (compound10b)), N-[3-(5-Chloro-2-ethoxy-6-methoxybenzoimidazol-1-yl) propyl]acetamide (compound 15a), N-[2-(2-methoxy-7,8-dihydro-6-oxa-1,3-diaza-as-indacen-1-yl)ethyl]acetamide (compound 19a), or a salt or derivative thereof, or a combination thereof. In some embodiments, the melatonin receptor agonist comprises Circadin®. In some embodiments, the melatonin receptor agonist comprises Slenyto®. In some embodiments, the melatonin receptor agonist comprises Ramelteon. In some embodiments, the melatonin receptor agonist comprises Tasimelteon. In some embodiments, the melatonin receptor agonist comprises Agomelatine. In some embodiments, the melatonin receptor agonist comprises TIK-301. In some embodiments, the melatonin receptor agonist comprises Piromelatine. In some embodiments, the melatonin receptor agonist comprises N-[2-(5-Chloro-2,6-dimethoxybenzoimidazol-1-yl)ethyl]acetamide. In some embodiments, the melatonin receptor agonist comprises N-[3-(5-Chloro-2-ethoxy-6-methoxybenzoimidazol-1-yl) propyl]acetamide. In some embodiments, the melatonin receptor agonist comprises N-[2-(2-methoxy-7,8-dihydro-6-oxa-1,3-diaza-as-indacen-1-yl)ethyl]acetamide. In some embodiments, the melatonin receptor agonist non-selectively activates type 1A (MT₁) and type 1B (MT₂) melatonin receptors. In some embodiments, the melatonin receptor agonist preferentially activates type 1A (MT₁) melatonin receptors. In some embodiments, the melatonin receptor agonist preferentially activates type 1B (MT₂) melatonin receptors. In some aspects, activating the melatonin receptor is a means to regulate activity of receptor interacting proteins, for example, TMEM33, CALR (calreticulin) and CNP (cyclic nucleotide phosphodiesterase). In some embodiments, the pharmaceutical composition is formulated as a gel, a cream, an ointment, a solution, a powder, a paste, or a foam. In some embodiments, the pharmaceutical composition is formulated to be delivered via a drug delivery device. In some embodiments, the pharmaceutical composition is formulated to be delivered via a vaginal ring, a vaginal tablet, a pessary, a suppository, a patch, or an intrauterine device. In some embodiments, the pharmaceutical composition is formulated for delayed release, sustained release, or extended release. In some aspects, the pharmaceutical composition is used in a timed-release device to achieve daily or nocturnal release of the melatonin receptor agonist. In some embodiments, the pharmaceutical composition comprises melatonin receptor agonist at an amount to be effective at least for 1 week, 2 weeks, 4 weeks, 2 months, or 6 months. In some aspects, the melatonin receptor agonist preferentially accumulates at the periphery compared to the central nervous system (CNS). In some aspects, the concentration of the melatonin receptor agonist is higher at the periphery (e.g. plasma concentration, peripheral tissues) compared to the CNS. In some aspects, the preferential accumulation of the melatonin receptor agonist at the periphery compared to the CNS is achieved by local administration (e.g. intravaginal, intrauterine). In some aspects, the preferential accumulation of the melatonin receptor agonist at the periphery compared to the CNS is due to the physical-chemical properties of the compound. In some aspects, the preferential accumulation of the melatonin receptor agonist at the periphery compared to the CNS is due to limited diffusion through the blood-brain barrier (BBB). In some aspects, the preferential accumulation of the melatonin receptor agonist at the periphery compared to the CNS is due to limited active transport through the BBB. In some aspects, the preferential accumulation of the melatonin receptor agonist at the periphery compared to the CNS is due to expulsion via BBB egress transporters (e.g., ABCB proteins). In some aspects, the preferential accumulation of the melatonin receptor agonist at the periphery compared to the CNS is due to increased metabolism or clearance of the melatonin receptor agonist in the CNS. In some aspects, the preferential accumulation of the melatonin receptor agonist at the periphery compared to the CNS is due to binding to proteins in the CNS that reduce the free concentration of the melatonin receptor agonist. In some aspects, the preferential accumulation of the melatonin receptor agonist at the periphery compared to the CNS is due to binding to proteins in the periphery. In some aspects, less than about 50%, 40%, 30%, 20%, 15%, or 10% of the pharmaceutical composition penetrates through the BBB of the subject. In some aspects, less than about 50%, 40%, 30%, 20%, 15%, or 10% of the melatonin receptor agonist penetrates through the BBB of the subject. In some aspects, decreased passive diffusion of the pharmaceutical composition through the BBB results in decreased CNS accumulation of the pharmaceutical composition. In some aspects, decreased active transport through the BBB results in decreased CNS accumulation of the pharmaceutical composition. In some aspects, increased egress from the BBB results in decreased CNS accumulation of the pharmaceutical composition. In some aspects, increased metabolism of an active component of a pharmaceutical composition results in decreased CNS accumulation of a melatonin receptor agonist. In some aspects, increased CNS clearance results in decreased CNS accumulation of the pharmaceutical composition. In some aspects, binding to protein in the CNS results in decreased CNS accumulation of the pharmaceutical composition. In some aspects, localization of administration of the pharmaceutical composition results in decreased CNS accumulation of the pharmaceutical composition. In some aspects, the melatonin receptor agonist is conjugated or encapsulated with a molecule that has a reduced capacity to cross the BBB. In some aspects, the melatonin receptor agonist is conjugated or encapsulated with a molecule that does not cross the blood brain barrier. In some aspects the molecule is hydrophilic, is a carbohydrate molecule, a peptide, or a synthetic molecule. In some aspects, the melatonin receptor agonist may have a reduced hydrophilicity compared to melatonin. In some aspects, the melatonin receptor agonist may have a reduced lipophilicity compared to melatonin.

Described herein, in certain aspects, are methods relating to the delivery of melatonin receptor agonists to tissues throughout the body of a subject. In some aspects, a method of treating or ameliorating a symptom of polycystic ovary syndrome (PCOS), endometriosis, chemotherapy-induced ovarian dysfunction (CIOD), or inflammation of reproductive tract in a subject in need thereof, comprises administering a composition comprising a melatonin receptor agonist to the subject, wherein the composition is administered via intravaginal, intrauterine, or systemic routes with preferential exposure at the periphery (e.g. higher concentration in plasma vs. CNS) of the subject. In some aspects, a method of preventing, reducing, or inhibiting one or more symptoms of CIOD in a subject in need thereof, comprises administering a composition comprising a melatonin receptor agonist to the subject, wherein the composition is administered via intravaginal, intrauterine, or systemic routes with preferential exposure at the periphery (e.g. higher concentration in plasma vs. CNS) of the subject. In some aspects, the melatonin receptor agonist comprises at least one of Circadin®, Slenyto®, Ramelteon, Tasimelteon, Agomelatine, TIK-301, Piromelatine, N-[2-(5-Chloro-2,6-dimethoxybenzoimidazol-1-yl)ethyl]acetamide (ACH000-143, (compound10b)), N-[3-(5-Chloro-2-ethoxy-6-methoxybenzoimidazol-1-yl) propyl]acetamide (compound 15a), N-[2-(2-methoxy-7,8-dihydro-6-oxa-1,3-diaza-as-indacen-1-yl)ethyl]acetamide (compound 19a), or a salt or derivative thereof, or a combination thereof. In some aspects, the melatonin receptor agonist non-selectively activates type 1A (MT₁) and type 1B (MT₂) melatonin receptors. In some aspects, the melatonin receptor agonist preferentially activates type 1A (MT₁) melatonin receptors. In some aspects, the melatonin receptor agonist preferentially activates type 1B (MT₂) melatonin receptors. In some aspects, the composition is formulated as a gel, a cream, an ointment, a solution, a powder, a paste, or a foam. In some aspects, the composition is administered by applying the composition on the dermal or transmucosal layer of the vaginal wall. In some aspects, the composition is administered via an intrauterine device. In some embodiments, the composition is formulated as to be delivered via a drug delivery device (e.g., a vaginal ring, a vaginal tablet, a pessary, a suppository, or a patch), wherein the composition is administered by placing the intravaginal device in close proximity to the subject's reproductive organ. In some aspects, the symptom comprises an abdominal pain, and the composition is administered in a dose and schedule effective to reduce the pain at least 20%, at least 30%, at least 40% within 24 hours, 48 hours, or 72 hours measured by a numerical rating scales (NRS). In some aspects, the composition is administered at least about every day, every 2 days, every 3 days, every 7 days, every 14 days, every 28 days, every 2 months. In some aspects, the administration of the melatonin receptor agonist has a limited effect (lower intensity and/or for shorter time) on melatonin receptor mediated CNS behavior (e.g., circadian rhythm, addiction, sleep behavior, motor control, memory extinction, etc.) compared to melatonin. In some aspects, the short half-life of the melatonin receptor agonist in the CNS allows a subject to experience improvements of symptoms mediated by CNS melatonin receptors, without experiencing side-effects caused by prolonged CNS exposure.

Provided herein are pharmaceutical compositions comprising a therapeutically effective amount of a melatonin receptor agonist, wherein the pharmaceutical composition is formulated for intravaginal, intrauterine, or systemic administration with reduced accumulation in central nervous system (CNS) compared to melatonin; wherein the therapeutically effective amount is effective to treat or ameliorate symptoms of polycystic ovary syndrome (PCOS), endometriosis, menopause, or inflammation of a reproductive tract in a subject in need thereof. In some aspects, the melatonin receptor agonist comprises at least one of Circadin®, Slenyto®, Ramelteon, Tasimelteon, Agomelatine, TIK-301, Piromelatine, N-[2-(5-Chloro-2,6-dimethoxybenzoimidazol-1-yl)ethyl]acetamide (ACH000-143, (compound10b)), N-[3-(5-Chloro-2-ethoxy-6-methoxybenzoimidazol-1-yl) propyl]acetamide (compound 15a), N-[2-(2-methoxy-7,8-dihydro-6-oxa-1,3-diaza-as-indacen-1-yl)ethyl]acetamide (compound 19a), or a salt or derivative thereof, or a combination thereof. In some aspects, the melatonin receptor agonist comprises Circadin®. In some aspects, the melatonin receptor agonist comprises Slenyto®. In some aspects, the melatonin receptor agonist comprises Ramelteon. In some aspects, the melatonin receptor agonist comprises Tasimelteon. In some aspects, the melatonin receptor agonist comprises Agomelatine. In some aspects, the melatonin receptor agonist comprises TIK-301. In some aspects, the melatonin receptor agonist comprises Piromelatine. In some aspects, the melatonin receptor agonist comprises N-[2-(5-Chloro-2,6-dimethoxybenzoimidazol-1-yl)ethyl]acetamide. In some aspects, the melatonin receptor agonist comprises N-[3-(5-Chloro-2-ethoxy-6-methoxybenzoimidazol-1-yl) propyl]acetamide. In some aspects, the melatonin receptor agonist comprises N-[2-(2-methoxy-7,8-dihydro-6-oxa-1,3-diaza-as-indacen-1-yl)ethyl]acetamide. In some aspects, the melatonin receptor agonist non-selectively activates type 1A (MT₁) and type 1B (MT₂) melatonin receptors. In some aspects, the melatonin receptor agonist preferentially activates type 1A (MT₁) melatonin receptors. In some aspects, the melatonin receptor agonist preferentially activates type 1B (MT₂) melatonin receptors. In some aspects, the pharmaceutical composition is formulated as a gel, an ointment, a solution, a powder, a paste, a foam, a cream or a lotion. In some aspects, the pharmaceutical composition is formulated to be delivered via a drug delivery device. In some aspects, the drug delivery device is a vaginal ring, a vaginal tablet, a pessary, a suppository, a patch, or an intrauterine device. In some aspects, the drug delivery device is configured to be placed in proximity to a reproductive organ of the subject. In some aspects, a physician or a qualified health professional may place the drug delivery device in proximity to a reproductive organ of the subject. In some embodiments, the drug delivery device is the intrauterine device and configured to be placed in proximity to a reproductive organ of the subject. In some aspects, the intrauterine device is configured for time-controlled release or remote-controlled release of one or a plurality of doses of the melatonin receptor agonist. In some aspects, the pharmaceutical composition is formulated for delayed release, sustained release, extended release, prolonged release, or slow release. In some aspects, the pharmaceutical composition comprises the melatonin receptor agonist at an amount to be effective at least about 1 week, 2 weeks, 4 weeks, 2 months, or 6 months. In some aspects, less than about 50%, 40%, 30%, 20%, 15%, 10% or 5% of the pharmaceutical composition accumulates in the CNS of the subject. In some embodiments, less than about 50%, 40%, 30%, 20%, 15%, 10% or 5% of Circadin® accumulates in the CNS of the subject. In some embodiments, less than about 50%, 40%, 30%, 20%, 15%, 10% or 5% of Slenyto® accumulates in the CNS of the subject. In some embodiments, less than about 50%, 40%, 30%, 20%, 15%, 10% or 5% of Ramelteon accumulates in the CNS of the subject. In some embodiments, less than about 50%, 40%, 30%, 20%, 15%, 10% or 5% of Tasimelteon accumulates in the CNS of the subject. In some embodiments, less than about 50%, 40%, 30%, 20%, 15%, 10% or 5% of Agomelatine accumulates in the CNS of the subject. In some embodiments, less than about 50%, 40%, 30%, 20%, 15%, 10% or 5% of TIK-301 accumulates in the CNS of the subject. In some embodiments, less than about 50%, 40%, 30%, 20%, 15%, 10% or 5% of Piromelatine accumulates in the CNS of the subject. In some embodiments, less than about 50%, 40%, 30%, 20%, 15%, 10% or 5% of N-[2-(5-Chloro-2,6-dimethoxybenzoimidazol-1-yl)ethyl]acetamide accumulates in the CNS of the subject. In some embodiments, less than about 50%, 40%, 30%, 20%, 15%, 10% or 5% of N-[3-(5-Chloro-2-ethoxy-6-methoxybenzoimidazol-1-yl) propyl]acetamide accumulates in the CNS of the subject. In some embodiments, less than about 50%, 40%, 30%, 20%, 15%, 10% or 5% of N-[2-(2-methoxy-7,8-dihydro-6-oxa-1,3-diaza-as-indacen-1-yl)ethyl]acetamide accumulates in the CNS of the subject. In some embodiments, the melatonin receptor agonist has a reduced rate of passive diffusion compared to melatonin. In some embodiments, the melatonin receptor agonist has an increased rate of cerebrospinal fluid (CSF) expulsion via blood-brain barrier (BBB) egress transporters compared to melatonin. In some embodiments, the melatonin receptor agonist has an increased rate of CNS metabolism compared to melatonin. In some embodiments, the melatonin receptor agonist has an increased rate of CNS clearance compared to melatonin. In some embodiments, the melatonin receptor agonist has increased binding to one or a plurality of CNS proteins compared to melatonin. In some embodiments, the melatonin receptor agonist has increased binding to a one or a plurality of peripheral proteins compared to melatonin. In some embodiments, the melatonin receptor agonist is conjugated or encapsulated with a molecule that has a low capacity to cross the blood brain barrier. In some embodiments, the molecule is a hydrophilic molecule, a carbohydrate molecule, a peptide, or a synthetic molecule. In some embodiments, pharmaceutical compositions described herein are for use in the treatment of polycystic ovary syndrome (PCOS) in a subject in need thereof. In some embodiments, pharmaceutical compositions described herein are for use in the treatment of endometriosis in a subject in need thereof. In some embodiments, pharmaceutical compositions described herein are for use in the treatment of symptoms of menopause in a subject in need thereof. In some embodiments, pharmaceutical compositions described herein are for use in the treatment of symptoms of chemotherapy-induced ovarian dysfunction (CIOD) in a subject in need thereof. In some embodiments, pharmaceutical compositions described herein are for use in the treatment of inflammation of a reproductive tract in a subject in need thereof. In some embodiments, pharmaceutical compositions described herein are for use in the treatment of symptoms of chemotherapy-induced ovarian failure (CIOF) in a subject in need thereof. In some embodiments, pharmaceutical compositions described herein are for use in the prevention of symptoms of CIOD in a subject in need thereof. In some embodiments, pharmaceutical compositions described herein are for use in the reduction of symptoms of CIOD in a subject in need thereof. In some embodiments, pharmaceutical compositions described herein are for use in the inhibition of symptoms of CIOD in a subject in need thereof.

Provided herein are methods of treating or ameliorating a symptom of polycystic ovary syndrome (PCOS), endometriosis, menopause, chemotherapy-induced ovarian dysfunction (CIOD), or inflammation of a reproductive tract in a subject in need thereof, comprising administering a pharmaceutical composition comprising a therapeutically effective amount of a melatonin receptor agonist, wherein the pharmaceutical composition is formulated for intravaginal, intrauterine, or systemic administration with reduced accumulation in central nervous system (CNS) compared to melatonin. In some embodiments, the pharmaceutical composition is administered intravaginally, intrauterine or systemically with reduced accumulation in the CNS of the subject compared to melatonin. In some embodiments, the pharmaceutical composition is administered intravaginally and comprises administering the pharmaceutical composition to a dermal or a transmucosal layer of a vaginal wall of the subject. In some embodiments, the symptom comprises an abdominal pain, a back pain, a chronic pelvic pain, a dysmenorrhea, an amenorrhea, an oligomenorrhea, glucose intolerance, insulin resistance, hyperandrogenemia, liver steatosis, hirsutism, infertility, weight gain, bone density loss, hair loss, hypertension or a combination thereof. In some embodiments, the administering comprises administration according to a dose and a schedule effective to reduce abdominal pain, back pain, chronic pelvic pain, dysmenorrhea, an amenorrhea, an oligomenorrhea, glucose intolerance, insulin resistance, hyperandrogenemia, liver steatosis, hirsutism, infertility, weight gain, bone density loss, hair loss, hypertension or a combination thereof at least 20%, at least 30%, at least 40% within 24 hours, 48 hours, or 72 hours measured by a numerical rating scales (NRS). In some embodiments, the administering comprises administration at least about every day, every 2 days, every 3 days, every 7 days, every 14 days, every 28 days, every 2 months, or every 6 months. In some embodiments, the administering comprises administration continuously for a duration of treatment. In some embodiments, the administering comprises administration at certain times of day or night according to a circadian rhythm of the subject. In some embodiments, the pharmaceutical composition regulates activity of melatonin receptors outside of the CNS according to the circadian rhythm of the subject. In some embodiments, the administering comprises administration at certain times of day or night irrespective of circadian fluctuations in plasma melatonin levels of the subject. In some embodiments, the administering does not disrupt a circadian rhythm of plasma melatonin levels. In some embodiments, the administering comprises administration at any time during a 24 hour period. In some embodiments, the administering has a reduced effect on a melatonin receptor mediated CNS behavior compared to administering an equivalent amount of melatonin. In some embodiments, the administering has a reduced effect on a melatonin receptor mediated CNS behavior during nighttime hours compared to administering an equivalent amount of melatonin. In some embodiments, the melatonin receptor mediated CNS behavior comprises circadian rhythm, addiction, sleep behavior, motor control, or memory extinction. In some aspects, the melatonin receptor agonist comprises at least one of Circadin®, Slenyto®, Ramelteon, Tasimelteon, Agomelatine, TIK-301, Piromelatine, N-[2-(5-Chloro-2,6-dimethoxybenzoimidazol-1-yl)ethyl]acetamide (ACH000-143, (compound10b)), N-[3-(5-Chloro-2-ethoxy-6-methoxybenzoimidazol-1-yl) propyl]acetamide (compound 15a), N-[2-(2-methoxy-7,8-dihydro-6-oxa-1,3-diaza-as-indacen-1-yl)ethyl]acetamide (compound 19a), or a salt or derivative thereof, or a combination thereof. In some embodiments, the melatonin receptor agonist comprises Circadin®. In some embodiments, the melatonin receptor agonist comprises Slenyto®. In some embodiments, the melatonin receptor agonist comprises Ramelteon. In some embodiments, the melatonin receptor agonist comprises Tasimelteon. In some embodiments, the melatonin receptor agonist comprises Agomelatine. In some embodiments, the melatonin receptor agonist comprises TIK-301. In some embodiments, the melatonin receptor agonist comprises Piromelatine. In some embodiments, the melatonin receptor agonist comprises N-[2-(5-Chloro-2,6-dimethoxybenzoimidazol-1-yl)ethyl]acetamide. In some embodiments, the melatonin receptor agonist comprises N-[3-(5-Chloro-2-ethoxy-6-methoxybenzoimidazol-1-yl) propyl]acetamide. In some embodiments, the melatonin receptor agonist comprises N-[2-(2-methoxy-7,8-dihydro-6-oxa-1,3-diaza-as-indacen-1-yl)ethyl]acetamide. In some embodiments, the melatonin receptor agonist non-selectively activates type 1A (MT₁) and type 1B (MT₂) melatonin receptors. In some embodiments, the melatonin receptor agonist preferentially activates type 1A (MT₁) melatonin receptors. In some embodiments, the melatonin receptor agonist preferentially activates type 1B (MT₂) melatonin receptors. In some embodiments, the pharmaceutical composition is formulated as a gel, an ointment, a solution, a powder, a paste, a foam, a cream or a lotion. In some embodiments, the pharmaceutical composition is formulated to be delivered via a drug delivery device. In some embodiments, the drug delivery device is a vaginal ring, a vaginal tablet, a pessary, a suppository, a patch, or an intrauterine device. In some embodiments, a physician or a qualified health professional may place the drug delivery device in proximity to a reproductive organ of the subject. In some embodiments, the administration comprises placing the drug delivery device in proximity to a reproductive organ of the subject. In some embodiments, the administration comprises placing the intrauterine device in proximity to a reproductive organ of the subject. In some embodiments, the intrauterine device is configured for time-controlled release or remote-controlled release of one or a plurality of doses of melatonin receptor agonist. In some embodiments, the pharmaceutical composition is formulated for delayed release, sustained release, extended release, prolonged release, or slow release. In some embodiments, the pharmaceutical composition comprises melatonin receptor agonist at an amount to be effective at least for 1 week, 2 weeks, 4 weeks, 2 months, or 6 months. In some embodiments, less than about 50%, 40%, 30%, 20%, 15%, 10% or 5% of the pharmaceutical composition accumulates in the CNS of the subject. In some embodiments, less than about 50%, 40%, 30%, 20%, 15%, 10% or 5% of Circadin® accumulates in the CNS of the subject. In some embodiments, less than about 50%, 40%, 30%, 20%, 15%, 10% or 5% of Slenyto® accumulates in the CNS of the subject. In some embodiments, less than about 50%, 40%, 30%, 20%, 15%, 10% or 5% of Ramelteon accumulates in the CNS of the subject. In some embodiments, less than about 50%, 40%, 30%, 20%, 15%, 10% or 5% of Tasimelteon accumulates in the CNS of the subject. In some embodiments, less than about 50%, 40%, 30%, 20%, 15%, 10% or 5% of Agomelatine accumulates in the CNS of the subject. In some embodiments, less than about 50%, 40%, 30%, 20%, 15%, 10% or 5% of TIK-301 accumulates in the CNS of the subject. In some embodiments, less than about 50%, 40%, 30%, 20%, 15%, 10% or 5% of Piromelatine accumulates in the CNS of the subject. In some embodiments, less than about 50%, 40%, 30%, 20%, 15%, 10% or 5% of N-[2-(5-Chloro-2,6-dimethoxybenzoimidazol-1-yl)ethyl]acetamide accumulates in the CNS of the subject. In some embodiments, less than about 50%, 40%, 30%, 20%, 15%, 10% or 5% of N-[3-(5-Chloro-2-ethoxy-6-methoxybenzoimidazol-1-yl) propyl]acetamide accumulates in the CNS of the subject. In some embodiments, less than about 50%, 40%, 30%, 20%, 15%, 10% or 5% of N-[2-(2-methoxy-7,8-dihydro-6-oxa-1,3-diaza-as-indacen-1-yl)ethyl]acetamide accumulates in the CNS of the subject. In some embodiments, the melatonin receptor agonist has a reduced rate of passive diffusion compared to melatonin. In some embodiments, the melatonin receptor agonist has an increased rate of cerebrospinal fluid (CSF) expulsion via BBB egress transporters compared to melatonin. In some embodiments, the melatonin receptor agonist has an increased rate of CNS metabolism compared to melatonin. In some embodiments, the melatonin receptor agonist has an increased rate of CNS clearance compared to melatonin. In some embodiments, the melatonin receptor agonist has increased binding to a one or a plurality of CNS proteins compared to melatonin. In some embodiments, the melatonin receptor agonist has increased binding to a one or plurality of peripheral proteins compared to melatonin. In some embodiments, the melatonin receptor agonist is conjugated or encapsulated with a molecule that has a low capacity to cross the blood brain barrier. In some embodiments, the molecule is a hydrophilic molecule, a carbohydrate molecule, a peptide, or a synthetic molecule.

Provided herein are uses of pharmaceutical compositions comprising a therapeutically effective amount of a melatonin receptor agonist, wherein the pharmaceutical composition is formulated for intravaginal, intrauterine, or systemic administration with reduced accumulation in central nervous system (CNS) compared to melatonin, for the manufacture of a medicament for treating polycystic ovary syndrome (PCOS) in a subject in need thereof. Provided herein are uses of pharmaceutical compositions comprising a therapeutically effective amount of a melatonin receptor agonist, wherein the pharmaceutical composition is formulated for intravaginal, intrauterine, or systemic administration with reduced accumulation in central nervous system (CNS) compared to melatonin, for the manufacture of a medicament for treating endometriosis in a subject in need thereof. Provided herein are uses of pharmaceutical compositions comprising a therapeutically effective amount of a melatonin receptor agonist, wherein the pharmaceutical composition is formulated for intravaginal, intrauterine, or systemic administration with reduced accumulation in central nervous system (CNS) compared to melatonin, for the manufacture of a medicament for treating symptoms of menopause in a subject in need thereof. Provided herein are uses of pharmaceutical compositions comprising a therapeutically effective amount of a melatonin receptor agonist, wherein the pharmaceutical composition is formulated for intravaginal, intrauterine, or systemic administration with reduced accumulation in central nervous system (CNS) compared to melatonin, for the manufacture of a medicament for treating inflammation of a reproductive tract in a subject in need thereof. Provided herein are uses of pharmaceutical compositions comprising a therapeutically effective amount of a melatonin receptor agonist, wherein the pharmaceutical composition is formulated for intravaginal, intrauterine, or systemic administration with reduced accumulation in central nervous system (CNS) compared to melatonin, for the manufacture of a medicament for treating chemotherapy-induced ovarian dysfunction (CIOD) in a subject in need thereof. In some embodiments, the medicament is prepared to be administered in a dosage regime comprising administering at least about every day, every 2 days, every 3 days, every 7 days, every 14 days, every 28 days, every 2 months, or every 6 months. In some embodiments, the medicament is prepared to be administered in a dosage regime comprising pretreatment that begins about 15 days, 14 days, 13 days, 12 days, 11 days, 10 days, 9 days, 8 days, 7 days, 6 days, 5 days, 4 days, 3 days, 2 days, 1 day, 18 hours, 12 hours, 8 hours, 6 hours, 4 hours, 2 hours, or 1 hour prior to initiation of a chemotherapy treatment in the subject. In some embodiments, the medicament is prepared to be administered in a dosage regime comprising administering continuously for a duration of treatment. In some embodiments, the medicament is prepared to be administered in a dosage regime comprising administering at certain times of day or night according to a circadian rhythm of the subject. In some embodiments, the medicament is prepared to be administered in a dosage regime comprising administering at certain times of day or night irrespective of circadian fluctuations in plasma melatonin levels. In some embodiments, the medicament is prepared to be administered in a dosage regime comprising administering that does not disrupt a circadian rhythm of plasma melatonin levels. In some embodiments, the medicament is prepared to be administered in a dosage regime comprising administering at any time during a 24 hour period. In some embodiments, the medicament is prepared to be administered in a dosage regime comprising administering having a reduced effect on a melatonin receptor mediated CNS behavior compared to administering an equivalent amount of melatonin. In some embodiments, the medicament is prepared to be administered in a dosage regime comprising administering having a reduced effect on a melatonin receptor mediated CNS behavior during nighttime hours compared to administering an equivalent amount of melatonin.

Provided herein are kits comprising pharmaceutical compositions described herein and instructions for use. In some embodiments, the instructions for use designate one or more indications in a subject in need of treatment, wherein the one or more indications comprise polycystic ovary syndrome (PCOS), endometriosis, menopause, chemotherapy-induced ovarian dysfunction (CIOD), chemotherapy-induced ovarian failure (CIOF), or inflammation of a reproductive tract, or a combination thereof. In some embodiments, the kit further comprises a drug delivery device for administering the pharmaceutical composition to a subject. In some embodiments, the drug delivery device is a vaginal ring, a vaginal tablet, a pessary, a suppository, a patch, or an intrauterine device. In some embodiments, the drug delivery device is configured to be placed in proximity to a reproductive organ of the subject. In some embodiments, the drug delivery device is the intrauterine device and configured to be placed in proximity to a reproductive organ of the subject. In some embodiments, the intrauterine device is configured for time-controlled release or remote-controlled release of one or a plurality of doses of the pharmaceutical composition formulated for delayed release, sustained release, extended release, prolonged release, or slow release.

### BRIEF DESCRIPTION OF THE DRAWINGS

FIG. 1 shows an illustration of primary phenotypic traits associated with polycystic ovary syndrome (PCOS), a subset of which are known to be impacted by MTNR1A or MTRN1B function.

FIG. 2A shows an interconnection diagram of the neuroendocrine, reproductive, and metabolic systems and cell types and hormones utilized by these systems in subjects with untreated PCOS.

FIG. 2B shows an interconnection diagram of the neuroendocrine, reproductive, and metabolic systems and cell types and hormones utilized by these systems in subjects with treated PCOS. Also shown are modulations that melatonin receptor agonist treatment would exhibit at various nodes.

The novel features of the disclosure are set forth with particularity in the appended claims. A better understanding of the features and advantages of the present disclosure will be obtained by reference to the following detailed description that sets forth illustrative embodiments.

### DETAILED DESCRIPTION

### Overview

In many instances, women would welcome and/or benefit from a therapy that treats polycystic ovary syndrome (PCOS) to relieve symptoms. In many instances, effective PCOS treatment of symptoms that also preserves or increases fertility would be preferred. PCOS phenotypic traits can affect numerous biological systems including the endocrine, metabolic, reproductive, and immune systems. Disruptions in the endocrine system in subjects with PCOS can affect organs including ovaries, adrenal glands, the pituitary gland, and the hypothalamus. Disruptions in the endocrine system in subjects with PCOS can lead to increased serum androgen levels, ovarian hyperandrogenism, high luteinizing hormone (LH) to follicle-stimulating hormone (FSH) ratios in the blood (high LH:FSH ratio), hirsutism, and acne. Abnormal interactions within the hypothalamic-pituitary-gonadal (HPG) axis can account for these disruptions in subjects with PCOS. Secondary effects of this HPG axis disruption can include adrenal hyperandrogenism and hypercortisolism. In some embodiments described herein, delivering melatonin receptor agonists to tissues with endocrine function can mitigate PCOS symptoms including ovarian hyperandrogenism, increased serum androgen levels, and hirsutism. In some embodiments described herein, delivering melatonin receptor agonists to tissues with metabolic function can mitigate PCOS symptoms including obesity and insulin resistance.

Disruptions in the reproductive system in subjects with PCOS primarily affect ovaries. Disruption of ovarian function in subjects with PCOS can lead to polycystic ovaries, oligoovulation, anovulation, low egg quality, low embryo quality, and infertility. Ovarian dysfunction can in turn affect the function of the endometrium. Endometrium dysfunction in PCOS can lead to phenotypes that include oligomenorrhea, amenorrhea, disrupted endometrial receptivity, and infertility. Melatonin is highly concentrated in ovarian follicular fluid and melatonin receptors are expressed in the ovary. Subjects with PCOS have increased melatonin levels in serum and decreased melatonin levels in ovarian follicular fluid. In some embodiments, delivering melatonin receptor agonists to ovarian tissue can mitigate PCOS symptoms including polycystic ovaries, oligoovulation, anovulation (correcting ovulatory disorders addresses amenorrhea or oligomenorrhea), low egg quality, low embryo quality, and infertility, including infertility due to disrupted interval of endometrial receptivity.

Disruptions in the cells of the immune system in subjects with PCOS affect the responsiveness of reproductive tissues as well as the response of multiple tissues to inflammation driven metabolic disturbances throughout the body. This immune system disruption in PCOS can present as an overactive immune response. Additionally, dysfunctional immune cells can impose low grade inflammation in tissues through the subject. In some instances, this low-grade inflammation is the result of increased production of proinflammatory cytokines secreted by dysfunctional immune cells. In some instances, prolonged inflammation of peripheral tissues programs imposes disruption of normal function of immune cells in PCOS patients that leads to increased oxidative stress in certain tissues. This imposed inflammation and increased oxidative stress has a deleterious effect on tissue and organ function and can lead to cellular damage. In some embodiments, delivering melatonin receptor agonists to immune cells and their progenitors throughout the body can mitigate PCOS symptoms including low-grade inflammation and also can reduce oxidative stress.

In many instances, women would welcome and/or benefit from a therapy that treats endometriosis to relieve symptoms. In some instances, an alternative to surgical removal of ectopic endometrial tissue would be preferred. In some instances, subjects with endometriosis present with endometriosis-associated chronic pelvic pain (EACPP). In some instances, subjects with endometriosis present with dysmenorrhea. In some instances, subjects with endometriosis present with back pain. In some instances, endometriosis can lead to decreased fertility or infertility. In some instances, endometriosis can lead to endometriosis-associated ovarian oxidative stress. In some instances, delivering melatonin receptor agonists to reproductive tissue and to sites near to, adjacent to, or proximally close to endometriosis lesions or sites of adenomyosis can mitigate the progression of endometriosis and help to alleviate debilitation and discomfort caused by EACPP, dysmenorrhea, back pain, or a combination thereof. In some instances, delivering melatonin receptor agonists to reproductive tissue and to sites near to, adjacent to, or proximally close to endometriosis lesions or sites of adenomyosis can also maintain or enhance fertility in a subject.

In many instances, women would welcome and/or benefit from a therapy that treats idiopathic inflammation of the reproductive tract. In some cases, acute or chronic inflammation of the reproductive tract can lead to ovulatory dysfunction, luteal insufficiency, increases in insulin resistance in ovarian tissue, hyperandrogenism, pre-eclampsia, disruptions in folliculogenesis, disruptions in ovarian progesterone production including luteal phase production of progesterone, reduced oocyte maturation, and reductions in successful assisted reproduction technology (ART) outcomes. In some instances, delivering melatonin receptor agonists to tissues of the reproductive tract can mitigate ovulatory dysfunction, luteal insufficiency, increases in insulin resistance in ovarian tissue, hyperandrogenism, pre-eclampsia, disruptions in folliculogenesis, disruptions in ovarian progesterone production including luteal phase production of progesterone, reduced oocyte maturation, and reductions in successful ART outcomes.

In many instances, women would welcome and/or benefit from a therapy that treats chemotherapy-induced ovarian dysfunction (CIOD). In some instances, CIOD can lead to hot flashes, osteoporosis, risk of infertility, infertility, sleep disturbances, joint pain, anxiety, depression, sexual dysfunction, increased risk of cardiovascular disease, loss of primordial ovarian follicles, accelerated activation of primordial ovarian follicles, follicular atresia, stromal tissue damage, ovarian vascular damage, ovarian inflammation, reduction in ovarian reserve, increase in rate of age-dependent loss of ovarian reserve, induction of fibrosis, early menopause, premature ovarian insufficiency, premature ovarian failure, induction of apoptosis in mature ovarian follicles, decreased estrogen production, decreased anti-müllerian hormone (AMH) production, follicular stimulating hormone (FSH) levels, decreased plasma levels of estradiol, or any combination thereof. In some instances, delivering melatonin receptor agonists to tissues of the reproductive tract can mitigate hot flashes, osteoporosis, risk of infertility, infertility, sleep disturbances, joint pain, anxiety, depression, sexual dysfunction, increased risk of cardiovascular disease, loss of primordial ovarian follicles, accelerated activation of primordial ovarian follicles, follicular atresia, stromal tissue damage, ovarian vascular damage, ovarian inflammation, reduction in ovarian reserve, increase in rate of age-dependent loss of ovarian reserve, induction of fibrosis, early menopause, premature ovarian insufficiency, premature ovarian failure, induction of apoptosis in mature ovarian follicles, decreased estrogen production, decreased anti-müllerian hormone (AMH) production, follicular stimulating hormone (FSH) levels, decreased plasma levels of estradiol, or any combination thereof.

In many instances, women would welcome and/or benefit from a therapy that prevents, reduces, or inhibits CIOD. In some instances, CIOD can lead to hot flashes, osteoporosis, risk of infertility, infertility, sleep disturbances, joint pain, anxiety, depression, sexual dysfunction, increased risk of cardiovascular disease, loss of primordial ovarian follicles, accelerated activation of primordial ovarian follicles, follicular atresia, stromal tissue damage, ovarian vascular damage, ovarian inflammation, reduction in ovarian reserve, increase in rate of age-dependent loss of ovarian reserve, induction of fibrosis, early menopause, premature ovarian insufficiency, premature ovarian failure, induction of apoptosis in mature ovarian follicles, decreased estrogen production, decreased anti-müllerian hormone (AMH) production, follicular stimulating hormone (FSH) levels, decreased plasma levels of estradiol, or any combination thereof. In some instances, delivering melatonin receptor agonists to tissues of the reproductive tract can mitigate a risk of developing, hot flashes, osteoporosis, infertility, sleep disturbances, joint pain, anxiety, depression, sexual dysfunction, cardiovascular disease, loss of primordial ovarian follicles, accelerated activation of primordial ovarian follicles, follicular atresia, stromal tissue damage, ovarian vascular damage, ovarian inflammation, reduction in ovarian reserve, increase in rate of age-dependent loss of ovarian reserve, induction of fibrosis, early menopause, premature ovarian insufficiency, premature ovarian failure, induction of apoptosis in mature ovarian follicles, decreased estrogen production, decreased anti-müllerian hormone (AMH) production, follicular stimulating hormone (FSH) levels, decreased plasma levels of estradiol, or any combination thereof following administration of a chemotherapy treatment. In some instances, delivering melatonin receptor agonists to tissues of the reproductive tract can prevent the development of hot flashes, osteoporosis, infertility, sleep disturbances, joint pain, anxiety, depression, sexual dysfunction, cardiovascular disease, loss of primordial ovarian follicles, accelerated activation of primordial ovarian follicles, follicular atresia, stromal tissue damage, ovarian vascular damage, ovarian inflammation, reduction in ovarian reserve, increase in rate of age-dependent loss of ovarian reserve, induction of fibrosis, early menopause, premature ovarian insufficiency, premature ovarian failure, induction of apoptosis in mature ovarian follicles, decreased estrogen production, decreased anti-müllerian hormone (AMH) production, follicular stimulating hormone (FSH) levels, decreased plasma levels of estradiol, or any combination thereof following administration of a chemotherapy treatment. In some instances, delivering melatonin receptor agonists to tissues of the reproductive tract can inhibit the development of hot flashes, osteoporosis, infertility, sleep disturbances, joint pain, anxiety, depression, sexual dysfunction, cardiovascular disease, loss of primordial ovarian follicles, accelerated activation of primordial ovarian follicles, follicular atresia, stromal tissue damage, ovarian vascular damage, ovarian inflammation, reduction in ovarian reserve, increase in rate of age-dependent loss of ovarian reserve, induction of fibrosis, early menopause, premature ovarian insufficiency, premature ovarian failure, induction of apoptosis in mature ovarian follicles, decreased estrogen production, decreased anti-müllerian hormone (AMH) production, follicular stimulating hormone (FSH) levels, decreased plasma levels of estradiol, or any combination thereof following administration of a chemotherapy treatment.

In some instances, delivering a melatonin receptor agonist to reproductive tissues can modulate (e.g., delay, inhibit, or prevent) ovarian senescence and/or control (e.g., delay, inhibit, or prevent) ovarian aging (e.g., ovarian senescence). In many instances, ovarian aging can be caused by time, genetics and/or environmental factors. In many instances, women with a genetic predisposition to develop a premature loss of ovarian reserve would welcome and/or benefit from a therapy that modulates (e.g., prevents, reduces, inhibits and/or delays folliculogenesis and/or decreases (e.g., prevents, reduces, inhibits and/or delays) the depletion of their ovarian reserve. In some instances, for example, women with mutations in genes critical for folliculogenesis and ovarian biology (e.g., AR, BMP15, ESR1, FIGLA, FMR1, FOXE1, FOXL2, FOXO3, FSHR, GALT, GDF9, INHA, NOBOX, NR5A1, SYCP2L, TGFBR3) would benefit from a therapy that controls (e.g., prevents, reduces, inhibits and/or delays) the depletion of their ovarian reserve.

### Pharmaceutical Compositions

Melatonin receptors are G-protein coupled, 7-transmembrane cell surface receptors that detect ligand outside the cell and can modulate intracellular responses based on ligand binding. Melatonin (the primary hormone secreted by the pineal gland and also produced in some extrapineal tissues) serves as the naturally produced high affinity ligand for melatonin receptors. Mammals have two types of melatonin receptors, termed MT₁ and MT₂. MT₁ is encoded by the MTNR1A locus and MT₂ is encoded by the MTNR1B locus. Both MT₁ and MT₂ have been shown to control a number of a melatonin-mediated biological functions including CNS regulation of circadian rhythms. MT₁ and MT₂ have been shown to be expressed in various parts of the CNS (suprachiasmatic nuclei, hippocampus, cerebellar cortex, prefrontal cortex, basal ganglia, substantia nigra, ventral tegmental area, nucleus accumbens and retinal horizontal, amacrine and ganglion cells). However, MT₁ and MT₂ are also expressed in cell types throughout the body (e.g., blood vessels, mammary gland, gastrointestinal tract, liver, kidney, bladder, ovary, testis, prostate, skin and the immune system) and can regulate the functions of various cell types, tissues and organs.

The high affinity MTNR1A (MT₁) and MTNR1B (MT₂) melatonin receptors are 350 and 362 amino acids long, respectively, with calculated molecular weights of 39-40 kDa. They are coupled to pertussis toxin-sensitive G proteins leading to the inhibition of adenylyl cyclase activity. MT₁ is a unique receptor relative to MT₂ as they show distinct molecular structures with only 61% amino acid identity and different chromosomal localization. They have two (MTNR1A) and one (MTNR1B) potential glycosylation sites in their N-terminus, and protein kinase C (PKC), casein kinase 1 (CK1), casein kinase 2 (CK2) and protein kinase A (PKA) phosphorylation sites which may participate in their regulation. Their structure consists of seven transmembrane (TM) helices (I-VII) linked by three alternating intracellular (IL1, IL2, and IL3) and extracellular (EL1, EL2, and EL3) loops. They differ from the other G protein-coupled receptor superfamily members as they have an Asparagine-Arginine-Tyrosine (NRY) motif, a variant of an Aspartic acid-Arginine-Tyrosine (DRY) (or glutamic acid-Arginine-Tyrosine (ERY)) that is present in intracellular loop II of all G protein-coupled receptors. This region is believed to be involved in signal transduction through G proteins. Mutation of Asparagine 124 in the NRY motif of the MT₁ melatonin receptor affects receptor trafficking and cell signaling. Melatonin receptors also have what appears to be a leucine zipper in TM IV, with 7 leucines in the MTNR1A and 6 leucines in the MTNR1B, which may be involved in protein-protein interactions. In MTNR1A Gly 20 (TM VI), Val 4 (TM IV), His 7 (TM IV), Ser 8 (TM III), and Ser 12 (TM III) are essential for melatonin binding. In MTNR1B, Cys 113 (in EL1) and Cys 190 (in EL2), two residues that are conserved in most GPCRs, are proposed to form a disulfide bond that is essential for high affinity melatonin binding.

The affinity of melatonin for both MT₁ and MT₂ is similar (Ki approximately 0.1 nM) despite significant structural differences between the two receptors (61% amino acid identity). MT₁ and MT₂ have overlapping roles in some biological functions and distinct roles in certain tissues or contexts. A shared element of activation of MT₁ and MT₂ includes ligand-binding mediated activation of Gᵢ proteins with inhibition of adenylyl cyclase and subsequent decrease of intracellular CAMP levels Distinct functions of MT₁ and MT₂ can be traced back to differences in tissue-specific expression patterns, differential levels of expression in certain cell types, differential localization and intracellular trafficking of MT₁ and MT₂ in certain cells, and distinct downstream signal transduction events. Both MT₁ and MT₂ can form homo-oligomers which facilitate melatonin-mediated signaling. MT₁ and MT₂ can also hetero-oligomerize to facilitate melatonin-mediated signaling. MT₁ and MT₂ have also been found to hetero-oligomerize with other receptors including the 5-HT_{2c} receptor. These different receptor complexes not only can have functional significance for signal diversification depending on cell type and context, but they also present an avenue for improved drug selectivity if one receptor subtype is selectively targeted or if a dual-targeting strategy has differential effects on the downstream activation of a specific receptor or specific receptor-complex.

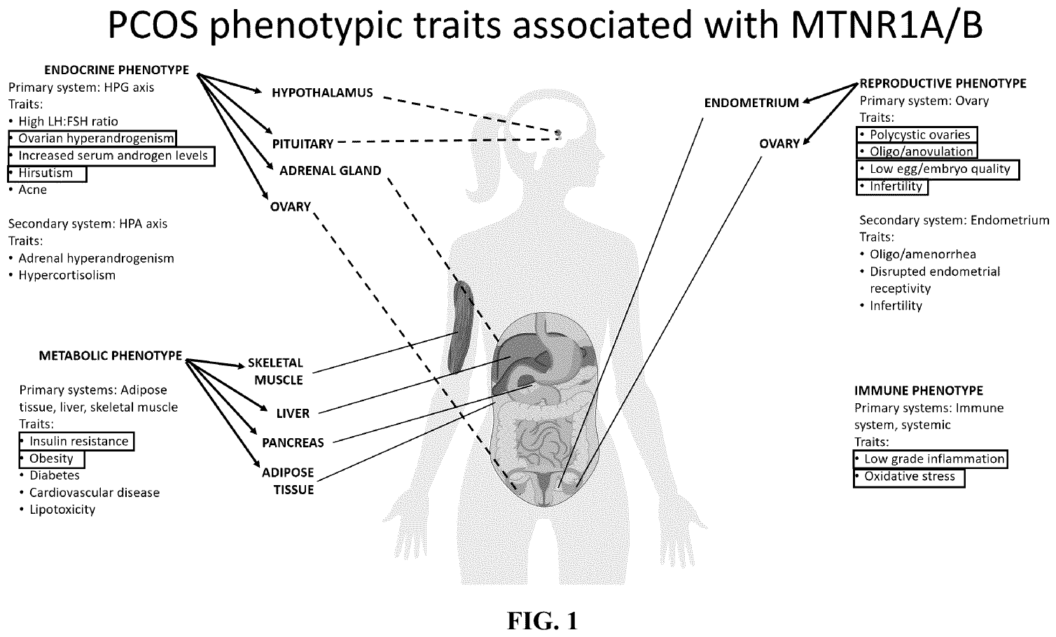

Polymorphisms in both MTNR1A and MTNR1B genes show association with PCOS in case-control studies (Yi S. et al. Biosci Rep. 2020 Jun. 26; 40 (6)) indicating roles for MTNR1A, MTNR1B, and the melatonin-signaling pathway in PCOS symptomatology and pathology. PCOS is an endocrine disorder in which a higher than normal number of small antral follicles form in the ovaries. As illustrated in FIG. 1, PCOS phenotypic traits that have been associated with MTNR1A and MTNR1B can be identified as affecting the endocrine, reproductive, immune, and metabolic systems. Boxes around specific traits that can be modulated through targeting the melatonin pathway within an affected system are shown in FIG. 1 (e.g., polycystic ovaries, oligoovulation, anovulation, low egg quality, low embryo quality, and infertility within the reproductive system). Subjects with PCOS can present with dysfunction in a variety of affected phenotypic traits in the endocrine, reproductive, immune, and metabolic systems. In addition, the extent of dysfunction of any particular phenotypic trait in a subject with PCOS can vary. In some instances, a subject with PCOS would benefit from a treatment that increases signaling through both MT₁ and MT₂ to a similar extent. In some instances, a subject with PCOS can benefit from a treatment that targets both MT₁ and MT₂. In some instances, a subject with PCOS can benefit from a treatment that preferentially targets MT₁ over MT₂. In some instances, a subject with PCOS can benefit from a treatment that preferentially targets MT₂ over MT₁. In some instances, a subject with PCOS can benefit from a treatment that exclusively targets MT₁. In some instances, a subject with PCOS can benefit from a treatment that exclusively targets MT₂. In some instances, the extent of preferentially targeting of MT₁ over MT₂ as an aspect of a treatment can be of additional benefit to a subject with PCOS. In some instances, the extent of preferentially targeting of MT₂ over MT₁ as an aspect of a treatment can be of additional benefit to a subject with PCOS.

Various melatonin receptor agonists have been identified and clinically evaluated. Table 1 and Table 2 list clinically developed melatonin receptor agonists, indications for use and properties of the molecules. Several melatonin receptor agonists (e.g., Circadin® and Slenyto®) contain melatonin as an active substance within a formulation. Several melatonin receptor agonists (e.g., Ramelteon, Tasimelteon, Agomelatine, TIK-301 and piromelatine) contain chemical analogs of melatonin as an active substance within a formulation. In some embodiments, a melatonin receptor agonist can function as a dual melatonin receptor agonist. In some embodiments, a dual melatonin receptor agonist is able to bind to both MT₁ and MT₂ with a high affinity. In some embodiments, a high affinity MT₁ and MT₂-binding dual melatonin receptor agonist can activate melatonin pathway signaling through both MT₁ and MT₂ at a concentration of the dual melatonin receptor agonist similar to a physiological concentration of melatonin found in an active signaling context. In some embodiments, a high affinity MT₁ and MT₂-binding dual melatonin receptor agonist can activate melatonin pathway signaling through both MT₁ and MT₂ at a concentration of the dual melatonin receptor agonist less than a physiological concentration of melatonin found in an active signaling context. In some embodiments, a high affinity MT₁ and MT₂-binding dual melatonin receptor agonist can activate melatonin pathway signaling through both MT₁ and MT₂ to a greater extent at a given concentration than the same concentration of melatonin. In some embodiments, a high affinity MT₁ and MT₂-binding dual melatonin receptor agonist can activate melatonin pathway signaling through both MT₁ and MT₂ to a lesser extent at a given concentration than the same concentration of melatonin. In some embodiments, a high affinity MT₁ and MT₂-binding dual melatonin receptor agonist can activate melatonin pathway signaling through MT₁ to a greater extent than can activate melatonin pathway signaling through MT₂. In some embodiments, a high affinity MT₁ and MT₂-binding dual melatonin receptor agonist can activate melatonin pathway signaling through MT₁ to a lesser extent than can activate melatonin pathway signaling through MT₂. In some embodiments, a high affinity MT₁ and MT₂-binding dual melatonin receptor agonist that can activate melatonin pathway signaling through MT₁ to a greater extent than can activate melatonin pathway signaling through MT₂ is preferred for treatment of a subject. In some embodiments, a high affinity MT₁ and MT₂-binding dual melatonin receptor agonist that can activate melatonin pathway signaling through MT₁ to a lesser extent than can activate melatonin pathway signaling through MT₂ is preferred for treatment of a subject.

In some aspects, a melatonin receptor agonist can function at less than full agonist activity toward a receptor. In some embodiments, a melatonin receptor agonist can function as a partial agonist. In some embodiments, a melatonin receptor agonist can function as a partial agonist to MT₁. In some embodiments, a melatonin receptor agonist can function as a partial agonist to MT₂. In some embodiments, a melatonin receptor agonist can exhibit high affinity binding for MT₁, MT₂, or both MT₁ and MT₂ and function as a partial agonist for MT₁, MT₂, or both MT₁ and MT₂. In some embodiments, a partial agonist for MT₁, MT₂, or both MT₁ and MT₂ may synergize with melatonin to increase overall agonism for MT₁, MT₂, or both MT₁ and MT₂. In some embodiments, a melatonin receptor agonist can exhibit function as an agonist or partial agonist for MT₁, MT₂, or both MT₁ and MT₂ and function as an antagonist for 5-HT_{2B}, or 5-HT_{2C}. In some embodiments, a melatonin receptor agonist that can exhibit function as an agonist or partial agonist for MT₁, MT₂, or both MT₁ and MT₂ and function as an antagonist for 5-HT_{2B}, or 5-HT_{2C} comprises agomelatine. In some embodiments, a melatonin receptor agonist can function as an antagonist and partial agonist (AT/PA). In some embodiments, the AT/PA function can occur simultaneously. In some embodiments, a melatonin receptor agonist can function as an AT/PA with at least 5%, 10%, 15%, 20%, 30%, 40%, 50%, 60%, 70%, 80%, 90% or 95% PA activity.

In some aspects, a melatonin receptor agonist has a binding affinity toward MT₁ or MT₂ that is similar to that of melatonin. In some aspects, a melatonin receptor agonist has a binding affinity toward MT₁ or MT₂ that is different than that of melatonin. In some embodiments, a melatonin receptor agonist has a binding affinity toward MT₁ that is less than at least 99%, 95%, 90%, 85%, 80%, 75%, 70%, 65%, 60%, 55%, 50%, 45%, 40%, 35%, 30%, 25%, 20%, 15%, 10%, 5%, 4%, 3%, 2%, 1%, 0.5%, 0.2%, 0.1%, 0.05%, 0.01%, 0.005%, 0.0001% that of melatonin toward MT₁. In some embodiments, a melatonin receptor agonist has a binding affinity toward MT₂ that is less than at least 99%, 95%, 90%, 85%, 80%, 75%, 70%, 65%, 60%, 55%, 50%, 45%, 40%, 35%, 30%, 25%, 20%, 15%, 10%, 5%, 4%, 3%, 2%, 1%, 0.5%, 0.2%, 0.1%, 0.05%, 0.01%, 0.005%, 0.0001% that of melatonin toward MT₂. In some embodiments, a melatonin receptor agonist has a binding affinity toward MT₁ that is greater than at least 2%, 3%, 4%, 5%, 6%, 7%, 8%, 9%, 10%, 12%, 14%, 15%, 17%, 20%, 22%, 25%, 27%, 30%, 35%, 40%, 45%, 50%, 60%, 70%, 80%, 90%, 100%, 110%, 125%, 150%, 175%, 200%, 250%, 300%, 350%, 400%, 450%, 500%, 600%, 700%, 800%, 900%, 1000%, 1250%, 1500%, 2000%, 3000%, 5000%, 7500%, or 10000% that of melatonin toward MT₁. In some embodiments, a melatonin receptor agonist has a binding affinity toward MT₂ that is greater than at least 2%, 3%, 4%, 5%, 6%, 7%, 8%, 9%, 10%, 12%, 14%, 15%, 17%, 20%, 22%, 25%, 27%, 30%, 35%, 40%, 45%, 50%, 60%, 70%, 80%, 90%, 100%, 110%, 125%, 150%, 175%, 200%, 250%, 300%, 350%, 400%, 450%, 500%, 600%, 700%, 800%, 900%, 1000%, 1250%, 1500%, 2000%, 3000%, 5000%, 7500%, or 10000% that of melatonin toward MT₂. In some embodiments, a melatonin receptor agonist having a binding affinity toward MT₁ that is greater than that of melatonin toward MT₁ is preferred as part of a treatment for a subject. In some embodiments, a melatonin receptor agonist having a binding affinity toward MT₂ that is greater than that of melatonin toward MT₂ is preferred as part of a treatment for a subject. In some embodiments, a melatonin receptor agonist having a binding affinity toward MT₁ that is less than that of melatonin toward MT₁ is preferred as part of a treatment for a subject. In some embodiments, a melatonin receptor agonist having a binding affinity toward MT₂ that is less than that of melatonin toward MT₂ is preferred as part of a treatment for a subject. In some embodiments, a melatonin receptor agonist having a binding affinity to MT₁ that is similar to that of melatonin to MT₁ and a biding affinity to MT₂ is greater than or less than that of melatonin to MT₂ is preferred as part of a treatment for a subject. In some embodiments, a melatonin receptor agonist having a binding affinity to MT₂ that is similar to that of melatonin to MT₂ and a biding affinity to MT₁ is greater than or less than that of melatonin to MT₁ is preferred as part of a treatment for a subject. In some embodiments, Circadin® or Slenyto® has an MT₁ binding affinity that is at least 20% greater than that of melatonin and an MT₂ binding affinity that is at least 70% less than that of melatonin. In some embodiments, Ramelteon has an MT₁ binding affinity that is at least 600% greater than that of melatonin and an MT₂ binding affinity that is similar than that of melatonin. In some embodiments, Tasimelteon has an MT₁ binding affinity that is at least 50% less than that of melatonin and an MT₂ binding affinity that is at least 25% greater than that of melatonin. In some embodiments, Agomelatine has an MT₁ binding affinity that is at least 90% greater than that of melatonin and an MT₂ binding affinity that is similar than that of melatonin. In some embodiments, TIK-301 has an MT₁ binding affinity that is at least 100% greater than that of melatonin and an MT₂ binding affinity that is at least 200% greater than that of melatonin. In some embodiments, Piromelatine has an MT₁ binding affinity that is at least 400% greater than that of melatonin and an MT₂ binding affinity that is at least 250% greater than that of melatonin. In some embodiments, Ramelteon or agomelatine have MT₁ and MT₂ binding affinities that are similar to that of melatonin to MT₁ and MT₂.

### Melatonin Receptor Agonists

In some aspects described herein, pharmaceutical compositions comprise a melatonin receptor agonist. In some instances, the melatonin receptor agonist comprises melatonin, Circadin®, Slenyto®, Ramelteon, Tasimelteon, Agomelatine, TIK-301, Piromelatine, any or salts or derivatives thereof or combinations thereof. In some instances, the melatonin receptor agonist is melatonin. In some instances, the melatonin receptor agonist is Circadin®. In some instances, the melatonin receptor agonist is Slenyto®. In some instances, the melatonin receptor agonist is Tasimelteon. In some instances, the melatonin receptor agonist is agomelatine. In some instances, the melatonin receptor agonist is beta-methyl-6-chloromelatonin (TIK-301). In some embodiments the melatonin receptor agonist is piromelatine. In some instances, the melatonin receptor agonist is N-(2-(5-chloro-2,6-dimethoxy-1H-benzo[d]imidazol-1-yl)ethyl) acetamide (ACH000-143, (compound10b). In some instances, the melatonin receptor agonist is N-(3-(5-chloro-2-ethoxy-6-methoxy-1H-benzo[d]imidazol-1-yl) propyl) acetamide (compound 15a). In some instances, the melatonin receptor agonist is N-2-(2-methoxy-7,8-dihydro-1H-benzofuro[4,5-d]imidazol-1-yl)ethyl) acetamide (compound 19a). In some instances, the melatonin receptor agonist is N-[2-(2-Ethoxy-7,8-dihydro-6-oxa-1,3-diaza-as-indacen-1-yl)ethyl]acetamide (compound 19b). In some instances, the melatonin receptor agonist is N-[2-(2-methoxy-7,8-dihydro-6-oxa-1,3-diaza-as-indacen-1-yl)ethyl]acetamide. In some instances, the melatonin receptor agonist is N-[3-(5-Chloro-2,6-dimethoxybenzoimidazol-1-yl) propyl]acetamide (compound 15b). In some instances, the melatonin receptor agonist is N-[2-(5-chloro-2-ethoxy-6-methoxybenzoimidazol-1-yl)ethyl]acetamide (compound 10a). In some instances, the melatonin receptor agonist is 2-(2-ethoxy-6-methoxybenzoimidazol-1-yl)ethylamine (compound 9). In some instances, the melatonin receptor agonist is N-[2-(2-Amino-5-methoxyphenylamino)ethyl]acetamide (compound 5a). In some instances, the melatonin receptor agonist is N-[2-(6-methoxy-2-methylsulfanylbenzoimidazol-1-yl)ethyl]acetamide (compound 7). In some instances, the melatonin receptor agonist is N-[2-(6-methoxy-2-oxo-2,3-dihydrobenzoimidazol-1-yl)ethyl]acetamide (compound 6). In some instances, the melatonin receptor agonist is N-[2-(5-methoxy-2-nitrophenylamino)ethyl]acetamide (compound 3a). In some instances, the melatonin receptor agonist is N-[2-(6-methoxybenzoimidazol-1-yl)ethyl]acetamide (compound 4). In some instances, the melatonin receptor agonist is N-[2-(2-Ethoxy-5-methoxybenzoimidazol-1-yl)ethyl]acetamide (compound 8j). In some instances, the melatonin receptor agonist is N-[3-(2,6-Dimethoxybenzoimidazol-1-yl) propyl]acetamide (compound 14b). In some instances, the melatonin receptor agonist is N-[3-(2-Ethoxy-6-methoxybenzoimidazol-1-yl) propyl]acetamide (compound 14a). In some instances, the melatonin receptor agonist is 4-oxo-4H-pyran-2-carboxylic acid [2-(2-ethoxy-6-methoxybenzoimidazol-1-yl)ethyl]amide (compound 8i). In some instances, the melatonin receptor agonist is Cyclohexanecarboxylic acid [2-(2-ethoxy-6-methoxybenzoimidazol-1-yl)ethyl]amide (compound 8h). In some instances, the melatonin receptor agonist is Cyclopentanecarboxylic acid [2-(2-ethoxy-6-methoxybenzoimidazol-1-yl)ethyl]amide (compound 8g). In some instances, the melatonin receptor agonist is Cyclobutanecarboxylic acid [2-(2-ethoxy-6-methoxybenzoimidazol-1-yl)ethyl]amide (compound 8f). In some instances, the melatonin receptor agonist is Cyclopropanecarboxylic acid [2-(2-ethoxy-6-methoxybenzoimidazol-1-yl)ethyl]amide (compound 8e). In some instances, the melatonin receptor agonist is N-[2-(2,6-Dimethoxybenzoimidazol-1-yl)ethyl]acetamide (compound 8d). In some instances, the melatonin receptor agonist is N-[2-(2,6-Dimethoxybenzoimidazol-1-yl)ethyl]acetamide (compound 8c). In some instances, the melatonin receptor agonist is N-[2-(2-Ethoxy-6-methoxybenzoimidazol-1-yl)ethyl]propionamide (compound 8b). In some instances, the melatonin receptor agonist is N-[2-(2-ethoxy-6-methoxybenzoimidazol-1-yl)ethyl]acetamide (compound 8a).

### Local Delivery of Pharmaceutical Compositions

In some aspects, the pharmaceutical composition delivery is by oral, transdermal, subcutaneous, intravenous or local administration. In some embodiments, the pharmaceutical composition of melatonin receptor agonist is delivered to the upper reproductive tract. In some embodiments, delivery to the upper reproductive tract allows for elevated local tissue concentration of melatonin receptor agonist without raising plasma concentration of melatonin receptor agonist to about the same extent. In some embodiments, delivery to the upper reproductive tract comprises a local tissue concentration of melatonin receptor agonist that is at least 1%, 2%, 3%, 4%, 5%, 6%, 7%, 8%, 9%, 10%, 12%, 14%, 15%, 16%, 18%, 20%, 22%, 25%, 27%, 30%, 32%, 35%, 37%, 40%, 45%, 50%, 55%, 60%, 65%, 70%, 75%, 80%, 85%, 90%, 95%, 100%, 110%, 120%, 130%, 140%, 150%, 175%, 200%, 250%, 300%, 350%, 400%, 450%, 500%, 600%, 700%, 800%, 900%, 1000%, 2000%, 3000%, 4000%, 5000%, or 10000% greater than that of plasma concentration of the melatonin receptor agonist. In some embodiments, the greater local tissue concentration of melatonin receptor agonist avoids the occurrence or extent of an unwanted drug response in the CNS or other organs of a subject. In some embodiments, the greater local tissue concentration of melatonin receptor agonist avoids the occurrence or extent of an unwanted drug response in the CNS or other organs of a subject and delivers a therapeutical effective amount of the melatonin receptor agonist to a target tissue. In some embodiments, local delivery of the pharmaceutical composition is by a drug delivery device. In some embodiments, local delivery of the pharmaceutical composition is by a vaginal gel, a vaginal ring, or an intrauterine device. In some embodiments, local delivery comprises local injection into a region nearby a target tissue. In some embodiments, the pharmaceutical composition administration can include injection or infusion, including intra-arterial, intracardiac, intradermal, intraduodenal, intramedullary, intramuscular, intraosseous, intraperitoneal, intravascular, intravenous, subcutaneous, inhalational, transdermal, transmucosal, sublingual, buccal and topical (including epicutaneous, dermal, enema, intranasal, vaginal) administration. In some exemplary embodiments, a route of administration can be via an injection such as an intramuscular, intravenous, subcutaneous, or intraperitoneal injection.

Solid dosage forms for oral administration can include capsules, tablets, caplets, pills, troches, lozenges, powders, and granules. A capsule can comprise a core material comprising a nutritive protein or composition and a shell wall that encapsulates a core material. In some embodiments a core material can comprise at least one of a solid, a liquid, and an emulsion. In some embodiments a shell wall material can comprise at least one of a soft gelatin, a hard gelatin, and a polymer. Suitable polymers can include but not limited to: cellulosic polymers such as hydroxypropyl cellulose, hydroxyethyl cellulose, hydroxypropyl methyl cellulose (HPMC), methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, such as those formed from acrylic acid, methacrylic acid, methyl acrylate, ammonio methylacrylate, ethyl acrylate, methyl methacrylate and/or ethyl methacrylate (e.g., those copolymers sold under the trade name “Eudragit”); vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac (purified lac). In some embodiments at least one polymer can function as taste-masking agents.

Tablets, pills, and the like can be compressed, multiply compressed, multiply layered, and/or coated. A coating can be single or multiple. In some embodiments, a coating material can comprise at least one of a saccharide, a polysaccharide, and glycoproteins extracted from at least one of a plant, a fungus, and a microbe. Non-limiting examples can include corn starch, wheat starch, potato starch, tapioca starch, cellulose, hemicellulose, dextrans, maltodextrin, cyclodextrins, inulins, pectin, mannans, gum arabic, locust bean gum, mesquite gum, guar gum, gum karaya, gum ghatti, tragacanth gum, funori, carrageenans, agar, alginates, chitosans, or gellan gum. In some embodiments a coating material can comprise a protein. In some embodiments, a coating material can comprise at least one of a fat and/or an oil. In some embodiments, the at least one of a fat and/or an oil can be high temperature melting. In some embodiments, the at least one of a fat and/or an oil can be hydrogenated or partially hydrogenated. In some embodiments, the at least one of a fat and/or an oil can be derived from a plant. In some embodiments, the at least one of a fat and/or an oil can comprise at least one of glycerides, free fatty acids, and fatty acid esters. In some embodiments, a coating material can comprise at least one edible wax. An edible wax can be derived from animals, insects, or plants. Non-limiting examples can include beeswax, lanolin, bayberry wax, carnauba wax, and rice bran wax. Tablets and pills can additionally be prepared with enteric coatings.

Liquid formulations can include a syrup (for example, an oral formulation), an intravenous formulation, an intranasal formulation, an ocular formulation (e.g. for treating an eye infection), an otic formulation (e.g. for treating an ear infection), an ointment, a cream, an aerosol, and the like. In some instances, a combination of various formulations can be administered. In some embodiments, a tablet, pill, and the like can be formulated for an extended release profile.

In some instances, a melatonin receptor agonist can be administered in a composition for topical administration. For topical administration, the melatonin receptor agonist may be formulated as is known in the art for direct application to a target area. Forms chiefly conditioned for topical application can take the form, for example, of creams, milks, gels, powders, dispersion or microemulsions, lotions thickened to a greater or lesser extent, impregnated pads, ointments or sticks, aerosol formulations (e.g. sprays or foams), hydrogel, soaps, detergents, lotions or cakes of soap. Other forms for this purpose include wound dressings, coated bandages or other polymer coverings, ointments, creams, lotions, pastes, jellies, sprays, and aerosols. Thus, a pharmaceutical composition disclosed herein can be delivered via patches or bandages for dermal administration. Alternatively, a pharmaceutical composition disclosed herein can be formulated to be part of an adhesive polymer, such as polyacrylate or acrylate/vinyl acetate copolymer. For long-term applications it might be desirable to use microporous and/or breathable backing laminates, so hydration or maceration of a skin can be minimized. A backing layer can be any appropriate thickness that will provide a desired protective and support functions. Topical administration may be in the form of a nail coating or lacquer.

Drops, such as eye drops or nose drops, may be formulated with one or more melatonin receptor agonist in an aqueous or non-aqueous base also comprising one or more dispersing agents, solubilizing agents or suspending agents. Liquid sprays can be pumped, or are conveniently delivered from pressurized packs. Drops can be delivered via a simple eye dropper-capped bottle, via a plastic bottle adapted to deliver liquid contents drop-wise, or via a specially shaped closure.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

A percentage by weight of a melatonin receptor agonist in a composition can depend on various factors. In some cases, a melatonin receptor agonist can be from about 0.01% to about 95%, from about 0.01% to about 90%, from about 0.01% to about 85%, from about 0.01% to about 80%, from about 0.01% to about 75%, from about 0.01% to about 70%, from about 0.01% to about 65%, from about 0.01% to about 60%, from about 0.01% to about 55%, from about 0.01% to about 50%, from about 0.01% to about 45%, from about 0.01% to about 40%, from about 0.01% to about 35%, from about 0.01% to about 30%, from about 0.01% to about 25%, from about 0.01% to about 20%, from about 0.01% to about 15%, from about 0.01% to about 10%, from about 0.01% to about 9%, from about 0.01% to about 8%, from about 0.01% to about 7%, from about 0.01% to about 6%, from about 0.01% to about 5%, from about 0.01% to about 4%, from about 0.01% to about 3%, from about 0.01% to about 2%, from about 0.01% to about 1%, from about 0.01% to about 0.9%, from about 0.01% to about 0.8%, from about 0.01% to about 0.7%, from about 0.01% to about 0.6%, from about 0.01% to about 0.5%, from about 0.01% to about 0.4%, from about 0.01% to about 0.3%, from about 0.01% to about 0.2%, or from about 0.01% to about 0.1% by weight with respect to a total weight of a composition.

In some embodiments, the pharmaceutical formulation can be in unit dose form. In some instances, a pharmaceutical formula can be lyophilized. In some exemplary embodiments, a pharmaceutical formulation can be stable for at least about 1 month, 2 months, 3 months, 4 months, 5 months, 6 months, 7 months, 8 months, 9 months, 10 months, 11 months, 1 year, 2 years, 3 years, 4 years, or 5 years when stored in a closed container at 10%, 20%, 30%, 40%, 50%, 60%, 70%, 80%, or 90% relative humidity at a temperature of from about 2° C. to about 30° C., from about 2° C. to about 29° C., from about 2° C. to about 28° C., from about 2° C. to about 27° C., from about 2° C. to about 26° C., from about 2° C. to about 25° C., from about 2° C. to about 24° C., from about 2° C. to about 23° C., from about 2° C. to about 22° C., from about 2° C. to about 21° C., from about 2° C. to about 20° C., from about 2° C. to about 19° C., from about 2° C. to about 18° C., from about 2° C. to about 17° C., from about 2° C. to about 16° C., from about 2° C. to about 15° C., from about 2° C. to about 14° C., from about 2° C. to about 13° C., from about 2° C. to about 12° C., from about 2° C. to about 11° C., from about 2° C. to about 10° C., from about 2° C. to about 9° C., from about 2° C. to about 8° C., from about 2° C. to about 7° C., from about 2° C. to about 6° C., from about 2° C. to about 5° C., from about 2° C. to about 4° C., or from about 2° C. to about 3° C. Stability can be determined by determined by an amount of melatonin receptor agonist remaining after a period of time. In some instances, at least about 10%, 20%, 30%, 40%, 50%, 60%, 70%, 80%, 90%, or 100% remains after a time period. In some cases, an amount of melatonin receptor agonist, salt, or metabolite remaining can be determined by: (a) loading a sample of a melatonin receptor agonist or salt thereof on an HPLC equipped with a size exclusion column that is at least 1, 2, 3, 4, 5, 6, 7, 8, 9, 10, 11, 12, 13, 14, 15, 16, 17, 18, 19, 20, 21, 22, 23, 24, 25, 26, 27, 28, 39, 30, 31, 32, 33, 34, 35, or 36 inches in length and can comprise a silica gel; and (b) performing mass spectroscopy on at least one sample eluted from a size exclusion column. In some cases, an amount of melatonin receptor agonist, salt, or metabolite remaining can be determined by performing an area under the curve (AUC) analysis of an HPLC chromatograph. In some cases, an amount of melatonin receptor agonist, salt, or metabolite remaining can be determined by performing an area under the curve (AUC) analysis of a mass spectra.

### Systemic Delivery of Pharmaceutical Compositions

In some aspects, the pharmaceutical composition delivery is by systemic delivery. In some embodiments, the systemic delivery comprises oral, transdermal, subcutaneous, intravenous or local administration. In some embodiments, the pharmaceutical composition has a diminished ability to accumulate in the CNS. In some embodiments, reduced accumulation in the CNS compared to periphery is caused by limited diffusion through the BBB and/or limited active transport through the BBB and/or expulsion via BBB egress transporters (e.g., ABCB proteins). In some embodiments, reduced accumulation in the CNS compared to periphery is caused by increased metabolism or clearance in the CNS of the melatonin receptor agonist. In some embodiments, reduced accumulation in the CNS compared to periphery is caused by binding to proteins that reduce the free concentration of the compound. In some embodiments, the pharmaceutical composition has a diminished ability to affect the CNS tissue. In some embodiments, the diminished accumulation in the CNS of the melatonin receptor agonist comprises at least 1%, 2%, 3%, 4%, 5%, 6%, 7%, 8%, 9%, 10%, 12%, 14%, 15%, 16%, 18%, 20%, 22%, 25%, 27%, 30%, 32%, 35%, 37%, 40%, 45%, 50%, 55%, 60%, 65%, 70%, 75%, 80%, 85%, 90%, 95%, 100%, 110%, 120%, 130%, 140%, 150%, 175%, 200%, 250%, 300%, 350%, 400%, 450%, 500%, 600%, 700%, 800%, 900%, 1000%, 2000%, 3000%, 4000%, 5000%, or 10000% reduction compared with melatonin's ability to accumulate in the CNS. In some embodiments, the reduced accumulation of the melatonin receptor agonist in the CNS after systemic administration manifests as a lower concentration in the CNS compared to the periphery and/or a shorter half-life in the CNS compared to the periphery. In some embodiments, the reduced accumulation of the melatonin receptor in the CNS after systemic administration manifests as a lower concentration of the melatonin receptor agonist in the CNS compared to the CNS concentration after systemic administration of an equivalent dose of melatonin.

In some aspects, the reduction in the melatonin receptor agonist ability to accumulate in the CNS avoids the occurrence or extent of an undesired drug response in the CNS of a subject. In some embodiments, the reduction in the melatonin receptor agonist ability to accumulate in the CNS avoids the occurrence or extent of an undesired drug response in the CNS of a subject and delivers a therapeutically effective amount of the melatonin receptor agonist to a target tissue. In some embodiments, a characteristic of the formulation of the pharmaceutical composition results in the reduction in the melatonin receptor agonist ability to accumulate in the CNS. In some embodiments, a modification to the melatonin receptor agonist results in the reduction in the melatonin receptor agonist ability to accumulate in the CNS. In some embodiments, the pharmaceutical composition comprises a melatonin receptor agonist-conjugate. In some embodiments, the conjugate comprises a hydrophilic molecule, a carbohydrate molecule, a peptide, or a synthetic molecule that inhibits or reduces the ability of the conjugate or the melatonin receptor agonist to cross the blood brain barrier. In some embodiments, diminished ability of the melatonin receptor agonist to accumulate in the CNS is due to decreased diffusion of the pharmaceutical composition across the BBB compared to an equivalent dose of melatonin. In some embodiments, diminished ability of the melatonin receptor agonist to accumulate in the CNS is due to decreased active transport of the pharmaceutical composition across the BBB compared to an equivalent dose of melatonin. In some embodiments, decreased active transport is through carrier- or receptor-mediated influx. In some embodiments, diminished ability of the melatonin receptor agonist to accumulate in the CNS is due to a higher extent of expulsion of the melatonin receptor agonist via BBB egress transporters compared expulsion of an equivalent dose of melatonin via BBB egress transporters.

In some aspects, when comparing a dose of a melatonin receptor agonist to an equivalent dose of melatonin, a comparison is made by comparing doses of melatonin receptor agonist and melatonin each at about the same molarity in a sample. In some embodiments, the comparison is made by comparing doses of Circadin® and melatonin each at about the same molarity in a sample. In some embodiments, the comparison is made by comparing doses of Slenyto® and melatonin each at about the same molarity in a sample. In some embodiments, the comparison is made by comparing doses of Ramelteon and melatonin each at about the same molarity in a sample. In some embodiments, the comparison is made by comparing doses of Tasimelteon and melatonin each at about the same molarity in a sample. In some embodiments, the comparison is made by comparing doses of Agomelatine and melatonin each at about the same molarity in a sample. In some embodiments, the comparison is made by comparing doses of TIK-301 and melatonin each at about the same molarity in a sample. In some embodiments, the comparison is made by comparing doses of Piromelatine and melatonin each at about the same molarity in a sample. In some embodiments, the comparison is made by comparing doses of N-[2-(5-Chloro-2,6-dimethoxybenzoimidazol-1-yl)ethyl]acetamide and melatonin each at about the same molarity in a sample. In some embodiments, the comparison is made by comparing doses of N-[3-(5-Chloro-2-ethoxy-6-methoxybenzoimidazol-1-yl) propyl]acetamide and melatonin each at about the same molarity in a sample. In some embodiments, the comparison is made by comparing doses of N-[2-(2-methoxy-7,8-dihydro-6-oxa-1,3-diaza-as-indacen-1-yl)ethyl]acetamide and melatonin each at about the same molarity in a sample. In some embodiments, when comparing a dose of a melatonin receptor agonist to an equivalent dose of melatonin, a comparison is made by comparing doses of melatonin receptor agonist and melatonin each at about the same weight percent in an equivalent sample. In some embodiments, an equivalent sample comprises the same ingredients except for the inclusion of melatonin or a melatonin receptor agonist.

In some aspects, the unique features of the melatonin receptor agonist can induce greater or lesser associations with known melatonin receptor interacting proteins. For example, the unique features may comprise alterations in associations with the recognized signaling pathways of the melatonin receptor through inhibitory G-protein coupling interactions that function to reduce cyclic adenosine monophosphate concentrations. In some embodiments, the unique features of the melatonin receptor agonist can induce greater or lesser associations with MT₁, MT₂, Mel1c, CAND2, VDR, QR2, MMP9, Pepsin, PP2A, PEPT1, PEPT2, GLUT1, Hyp-1, LLPR-10.2B, mtPTP, serum albumin, CaM, Calreticulin, or any combination thereof.

### Pharmacokinetic Profile

Various melatonin receptor agonists with different pharmacokinetic characteristics are contemplated herein. In some aspects, a melatonin receptor agonist has had a binding affinity to a receptor measured in vitro. In some embodiments, the receptor comprises MT₁, MT₂, 5-HT_{2B}, or 5-HT_{2C}. In some instances, a melatonin receptor agonist's binding affinity for a specific receptor has been measured and expressed as an inhibition constant (Kᵢ) value. In some embodiments, the Kᵢ value represents the binding affinity for a melatonin receptor agonist to a specific receptor in vivo. Table 1 and Table 2 list calculated Kᵢ values for melatonin receptor agonists towards MT₁, MT₂, 5-HT_{2B}, or 5-HT_{2C}.

In some aspects, the pharmaceutical composition described herein has a desirable pharmacokinetic (PK) profile within a subject when administered to the subject. In some embodiments, PK profile comprises a lower level of melatonin receptor agonist bioavailability in the CNS than in peripheral non-CNS tissues. In some embodiments, the PK profile comprises a higher level of melatonin receptor agonist bioavailability near target cells outside of the CNS. In some embodiments, the PK profile comprises a lower level of melatonin receptor agonist bioavailability in the CNS than near target cells outside of the CNS. In some embodiments, the PK profile comprises sufficient melatonin receptor agonist bioavailability near target cells outside of the CNS to provide a therapeutically effective treatment and melatonin receptor agonist bioavailability in the CNS at a level that does not induce undesired drug response in the CNS. In some embodiments, the target cells outside of the CNS comprise cells involved in endocrine function, cells involved in reproductive function, cells involved in metabolic function, or cells involved in immune function. In some embodiments, the target cells outside of the CNS comprise cells of the upper reproductive tract. In some embodiments, the cells of the upper reproductive tract comprise ovarian cells, egg cells, Fallopian tube cells, uterine cells, endometrial cells, myometrial cells, or cervical cells. In some embodiments, the cells involved in reproductive function comprise ovarian cells, egg cells, Fallopian tube cells, uterine cells, endometrial cells, myometrial cells, or cervical cells. In some embodiments, the target cells outside of the CNS involved in endocrine function comprise cells of the adrenal glands, pancreatic cells, thyroid gland cells, parathyroid gland cells, or ovarian cells. In some embodiments, the target cells outside of the CNS involved in metabolic function comprise skeletal muscle cells, liver cells, pancreatic cells, or adipose tissue cells. In some embodiments, the target cells outside of the CNS involved in immune function comprise lymphocytes, neutrophils, monocytes, or macrophages. In some embodiments, the target cells outside of the CNS involved in immune function comprise the progenitors of lymphocytes, neutrophils, monocytes, or macrophages. In some embodiments, the lymphocytes comprise T-cells, B-cells, or NK cells.

Described herein, in certain aspects, are pharmaceutical compositions comprising a PK profile within a subject that delivers an effective amount of melatonin receptor agonist molecules to target cells outside of the CNS. In some embodiments, the PK profile does not raise the level of bioavailability of melatonin receptor agonists within the CNS to an extent that the pharmaceutical composition induces somnolence in the subject. In some embodiments, the PK profile indicates that melatonin receptor agonist within the CNS does not accumulate to an extent that the pharmaceutical composition induces somnolence in the subject. In some embodiments, the PK profile does not raise the level of bioavailability of melatonin receptor agonists within the CNS to an extent that the pharmaceutical composition induces sleep. In some embodiments, the PK profile does not raise the level of bioavailability of melatonin receptor agonists within the CNS to an extent that the pharmaceutical composition disrupts the natural circadian rhythms of the subject. In some embodiments, the PK profile does not raise the level of bioavailability of melatonin receptor agonist within the CNS to an extent that the pharmaceutical composition disrupts the function of the HPG axis. In some embodiments, the PK profile does not raise the level of bioavailability of melatonin receptor agonist within the CNS to an extent that the pharmaceutical composition produces adverse neurological effects. In some embodiments, the adverse neurological effect may be a change in addiction in the subject. In some embodiments, the PK profile does not raise the level of bioavailability of melatonin receptor agonist within the CNS to an extent that the pharmaceutical composition produces adverse behavioral effects. In some embodiments, the PK profile does not raise the level of bioavailability of melatonin receptor agonist in plasma to elicit CNS effects. In some embodiments, the PK profile does not raise the level of bioavailability of melatonin receptor agonist within the CNS to an extent that the pharmaceutical composition disrupts sleep behavior, circadian rhythm, cognitive function, processing of sensory information, motor control, memory extinction, or other neuronal functions of the CNS. In some embodiments, the PK profile of the pharmaceutical composition maintains levels of the melatonin receptor agonist in plasma of the subject below about 20, 19.5, 19, 18.5, 18, 17.5, 17, 16.5, 16, 15.5, 15, 14.5, 14, 13.5, 13, 12.5, 12, 11.5, 11, 10.5, 10, 9.8, 9.6, 9.4, 9.2, 9.0, 8.8, 8.6, 8.4, 8.2, 8.0, 7.8, 7.6, 7.4, 7.2, 7.0, 6.8, 6.6, 6.4, 6.2, 6.0, 5.8, 5.6, 5.4, 5.2, 5.0, 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.95, 0.9, 0.85, 0.8, 0.75, 0.7, 0.65, 0.6, 0.55, 0.5, 0.45, 0.4, 0.35, 0.30, 0.25, 0.2, 0.19, 0.18, 0.17, 0.16, 0.15, 0.14, 0.13, 0.12, 0.11, 0.1, 0.09, 0.08, 0.07, 0.06, 0.05, 0.04, 0.03, 0.02, 0.01, 0.005, 0.001, 0.0005, or 0.0001 pg/mL.

In some aspects, the melatonin receptor agonist has a half-life (t½) that is greater than that of melatonin. In some embodiments, the t 1, of the melatonin receptor agonist is at least 2.5%, 5%, 10%, 15%, 20%, 25%, 30%, 35%, 40%, 45%, 50%, 60%, 70%<80%, 90%, 100%, 125%, 150%, 200%, 300%, 400%, 500%, 600%, 800%, 1000%, 1250%, 1500%, 2000%, 3000%, 5000%, 10000%, 50000%, or 100000% greater than that of melatonin. In some embodiments, the t½ of Circadin®, Slenyto®, Ramelteon, Tasimelteon, Agomelatine, TIK-301, Piromelatine, N-[2-(5-Chloro-2,6-dimethoxybenzoimidazol-1-yl)ethyl]acetamide (ACH000-143, (compound10b)), N-[3-(5-Chloro-2-ethoxy-6-methoxybenzoimidazol-1-yl) propyl]acetamide (compound 15a), N-[2-(2-methoxy-7,8-dihydro-6-oxa-1,3-diaza-as-indacen-1-yl)ethyl]acetamide (compound 19a) is greater than that of melatonin. In some embodiments, the plasma t½ of the melatonin receptor agonist is greater than the CNS t½. Described herein, in certain embodiments, are pharmaceutical compositions comprising a PK profile within a subject that delivers an effective amount of melatonin receptor agonist to target cells outside of the CNS. In some embodiments, the PK profile comprises a more continuous bioavailability of melatonin receptor agonist outside of the CNS throughout a 24-hour period than compared to the inherent diurnal pattern of melatonin plasma concentrations in the subject. In some embodiments, the area under the curve (AUC) calculation for combined melatonin receptor agonist delivered outside the CNS plus naturally produced melatonin comprises a larger value than the AUC calculation for naturally produced melatonin alone in the subject. In some embodiments, the AUC calculation for melatonin receptor agonist delivered outside the CNS will be measured over a time period of 1, 2, 3, 4, 5, 6, 7, 8, 9, 10, 11, 12, 13, 14, 15, 16, 17, 18, 19, 20, 21, 22, 23, 24, 26, 28, 30, 32, 34, 36, 40, 48, 56, 64, 72, 84, 96, 108, or 120 hours. In some embodiments, the AUC calculation for melatonin receptor agonist delivered outside the CNS will be at least 1%, 2%, 3%, 4%, 5%, 6%, 7%, 8%, 9%, 10%, 11%, 12%, 13%, 14%, 15%, 16%, 17%, 18%, 19%, 20%, 22%, 24%, 26%, 28%, 30%, 32%, 34%, 36%, 38%, 40%, 43%, 46%, 49%, 52%, 55%, 58%, 61%, 64%, 67%, 70%, 75%, 80%, 85%, 90%, 95%, 100%, 110%, 120%, 130%, 140%, 150%, 160%, 170%, 180%, 200%, 250%, 300%, 350%, 400%, 450%, 500%, 600%, 700%, 800%, 900%, or 1000% the value of the AUC calculation for naturally produced melatonin in the subject. In some embodiments, the PK profile of melatonin receptor agonist comprises a delayed-release, a sustained-release, or an extended-release profile. In some embodiments, the melatonin receptor agonist is released from its formulation with first order kinetics. In some embodiments, the melatonin receptor agonist is released from its formulation with zero order kinetics.

Described herein, in certain aspects, are pharmaceutical compositions comprising a PK profile within a subject that delivers melatonin receptor agonists to target cells outside of the CNS. In some embodiments, the PK profile allows for the melatonin receptor agonists to non-selectively activate MTNR1A and MTNR1B in a plurality of target cells outside of the CNS. In some embodiments, the plurality of target cells outside the CNS comprise endocrine, reproductive, metabolic, or immune cells, or any combination thereof. In some embodiments, the endocrine or reproductive cells are ovarian cells. In some embodiments, the PK profile allows for the melatonin receptor agonists to preferentially activate type 1A (MT₁) melatonin receptors. MT₁ receptors are produced from translation of MTNR1A transcripts. In some embodiments, the PK profile allows for the melatonin receptor agonists to preferentially activate type 1B (MT₂) melatonin receptors. MT₂ receptors are produced from translation of MTNR1B transcripts.

Described herein, in certain aspects, are pharmaceutical compositions comprising a PK profile within a subject that delivers melatonin receptor agonist to a target tissue outside of the CNS. In some embodiments, the pharmaceutical composition has a reduced ability to penetrate the BBB. In some embodiments, the pharmaceutical composition has a reduced ability to be actively transported through the BBB. In some embodiments, the pharmaceutical composition has an ability to be expelled via BBB egress transporters (e.g., ABCB proteins). In some embodiments, the pharmaceutical composition is expelled via BBB egress transporters to a greater extent than an equivalent dose of systemic melatonin. In some embodiments, the pharmaceutical composition has increased metabolism and/or clearance of the compound in the CNS. In some embodiments, the pharmaceutical composition has the ability to bind to proteins in the CNS that reduce the free concentration of the compound. In some embodiments, less than about 50%, 45%, 40%, 35%, 30%, 25%, 22%, 20%, 18%, 15%, 13%, 10%, 9%, 8%, 7%, 6%, 5%, 4.5%, 4%, 3.5%, 3%, 2.5%, 2%, 1.5%, 1%, 0.75%, 0.5%, 0.3%, 0.2%, 0.1%, 0.05%, 0.02%, 0.01%, 0.005%, or 0.001% of the pharmaceutical composition accumulates in the CNS of the subject. In some embodiments, less than about 50%, 45%, 40%, 35%, 30%, 25%, 22%, 20%, 18%, 15%, 13%, 10%, 9%, 8%, 7%, 6%, 5%, 4.5%, 4%, 3.5%, 3%, 2.5%, 2%, 1.5%, 1%, 0.75%, 0.5%, 0.3%, 0.2%, 0.1%, 0.05%, 0.02%, 0.01%, 0.005%, or 0.001% of the melatonin receptor agonist accumulates in the CNS of the subject. In some embodiments, less than about 50%, 45%, 40%, 35%, 30%, 25%, 22%, 20%, 18%, 15%, 13%, 10%, 9%, 8%, 7%, 6%, 5%, 4.5%, 4%, 3.5%, 3%, 2.5%, 2%, 1.5%, 1%, 0.75%, 0.5%, 0.3%, 0.2%, 0.1%, 0.05%, 0.02%, 0.01%, 0.005%, or 0.001% of the pharmaceutical composition diffuse through the BBB. In some embodiments, less than about 50%, 45%, 40%, 35%, 30%, 25%, 22%, 20%, 18%, 15%, 13%, 10%, 9%, 8%, 7%, 6%, 5%, 4.5%, 4%, 3.5%, 3%, 2.5%, 2%, 1.5%, 1%, 0.75%, 0.5%, 0.3%, 0.2%, 0.1%, 0.05%, 0.02%, 0.01%, 0.005%, or 0.001% of the melatonin receptor agonist diffuse through the BBB. In some embodiments, less than about 50%, 45%, 40%, 35%, 30%, 25%, 22%, 20%, 18%, 15%, 13%, 10%, 9%, 8%, 7%, 6%, 5%, 4.5%, 4%, 3.5%, 3%, 2.5%, 2%, 1.5%, 1%, 0.75%, 0.5%, 0.3%, 0.2%, 0.1%, 0.05%, 0.02%, 0.01%, 0.005%, or 0.001% of the pharmaceutical composition is actively transported through the BBB. In some embodiments, less than about 50%, 45%, 40%, 35%, 30%, 25%, 22%, 20%, 18%, 15%, 13%, 10%, 9%, 8%, 7%, 6%, 5%, 4.5%, 4%, 3.5%, 3%, 2.5%, 2%, 1.5%, 1%, 0.75%, 0.5%, 0.3%, 0.2%, 0.1%, 0.05%, 0.02%, 0.01%, 0.005%, or 0.001% of the melatonin receptor agonist is actively transported through the BBB. In some embodiments, more than about 80%, 75%, 70%, 65%, 60%, 50%, 45%, 40%, 35%, 30%, 25%, 22%, 20%, 18%, 15%, 13%, 10%, 9%, 8%, 7%, 6%, 5%, 4.5%, 4%, 3.5%, 3%, 2.5%, 2%, 1.5%, 1%, 0.75%, 0.5%, 0.3%, 0.2%, 0.1%, 0.05%, 0.02%, 0.01%, 0.005%, or 0.001% of the pharmaceutical composition is expelled via BBB egress transporters (e.g., ABCB proteins). In some embodiments, more than about 80%, 75%, 70%, 65%, 60%, 50%, 45%, 40%, 35%, 30%, 25%, 22%, 20%, 18%, 15%, 13%, 10%, 9%, 8%, 7%, 6%, 5%, 4.5%, 4%, 3.5%, 3%, 2.5%, 2%, 1.5%, 1%, 0.75%, 0.5%, 0.3%, 0.2%, 0.1%, 0.05%, 0.02%, 0.01%, 0.005%, or 0.001% of the melatonin receptor agonist is expelled via BBB egress transporters (e.g., ABCB proteins). In some embodiments, more than about 80%, 75%, 70%, 65%, 60%, 50%, 45%, 40%, 35%, 30%, 25%, 22%, 20%, 18%, 15%, 13%, 10%, 9%, 8%, 7%, 6%, 5%, 4.5%, 4%, 3.5%, 3%, 2.5%, 2%, 1.5%, 1%, 0.75%, 0.5%, 0.3%, 0.2%, 0.1%, 0.05%, 0.02%, 0.01%, 0.005%, or 0.001% of the pharmaceutical composition is metabolized in the CNS. In some embodiments, more than about 80%, 75%, 70%, 65%, 60%, 50%, 45%, 40%, 35%, 30%, 25%, 22%, 20%, 18%, 15%, 13%, 10%, 9%, 8%, 7%, 6%, 5%, 4.5%, 4%, 3.5%, 3%, 2.5%, 2%, 1.5%, 1%, 0.75%, 0.5%, 0.3%, 0.2%, 0.1%, 0.05%, 0.02%, 0.01%, 0.005%, or 0.001% of the melatonin receptor agonist is metabolized in the CNS. In some embodiments, more than about 80%, 75%, 70%, 65%, 60%, 50%, 45%, 40%, 35%, 30%, 25%, 22%, 20%, 18%, 15%, 13%, 10%, 9%, 8%, 7%, 6%, 5%, 4.5%, 4%, 3.5%, 3%, 2.5%, 2%, 1.5%, 1%, 0.75%, 0.5%, 0.3%, 0.2%, 0.1%, 0.05%, 0.02%, 0.01%, 0.005%, or 0.001% of the pharmaceutical composition is bound by proteins in the CNS that reduce the free concentration of the melatonin receptor agonist. In some embodiments, more than about 80%, 75%, 70%, 65%, 60%, 50%, 45%, 40%, 35%, 30%, 25%, 22%, 20%, 18%, 15%, 13%, 10%, 9%, 8%, 7%, 6%, 5%, 4.5%, 4%, 3.5%, 3%, 2.5%, 2%, 1.5%, 1%, 0.75%, 0.5%, 0.3%, 0.2%, 0.1%, 0.05%, 0.02%, 0.01%, 0.005%, or 0.001% of the melatonin receptor agonist is bound by proteins in the CNS that reduce the free concentration of the melatonin receptor agonist.

In some cases, a melatonin receptor agonist, salt thereof, or pharmaceutical composition comprising a melatonin receptor agonist or salt thereof described herein can be administered at a dose of from about 0.1 to about 1000 mg, from about 1 mg to about 1000 mg, from about 5 mg to about 1000 mg, from about 10 mg to about 1000 mg, from about 15 mg to about 1000 mg, from about 20 mg to about 1000 mg, from about 25 mg to about 1000 mg, from about 30 mg to about 1000 mg, from about 35 mg to about 1000 mg, from about 40 mg to about 1000 mg, from about 45 mg to about 1000 mg, from about 50 mg to about 1000 mg, from about 55 mg to about 1000 mg, from about 60 mg to about 1000 mg, from about 65 mg to about 1000 mg, from about 70 mg to about 1000 mg, from about 75 mg to about 1000 mg, from about 80 mg to about 1000 mg, from about 85 mg to about 1000 mg, from about 90 mg to about 1000 mg, from about 95 mg to about 1000 mg, from about 100 mg to about 1000 mg, from about 150 mg to about 1000 mg, from about 200 mg to about 1000 mg, from about 250 mg to about 1000 mg, from about 300 mg to about 1000 mg, from about 350 mg to about 1000 mg, from about 400 mg to about 1000 mg, from about 450 mg to about 1000 mg, from about 500 mg to about 1000 mg, from about 550 mg to about 1000 mg, from about 600 mg to about 1000 mg, from about 650 mg to about 1000 mg, from about 700 mg to about 1000 mg, from about 750 mg to about 1000 mg, from about 800 mg to about 1000 mg, from about 850 mg to about 1000 mg, from about 900 mg to about 1000 mg, or from about 950 mg to about 1000 mg.

In some cases, a melatonin receptor agonist, salt thereof, or pharmaceutical composition comprising a melatonin receptor agonist or salt thereof described herein can be administered at a dose of from about 0.1 to about 60 mg, from about 0.5 mg to about 60 mg, from about 1 mg to about 60 mg, from about 2 mg to about 60 mg, from about 3 mg to about 60 mg, from about 4 mg to about 60 mg, from about 5 mg to about 60 mg, from about 8 mg to about 60 mg, from about 10 mg to about 60 mg, from about 15 mg to about 60 mg, from about 20 mg to about 60 mg, from about 25 mg to about 60 mg, from about 30 mg to about 60 mg, or from about 40 mg to about 60 mg.

In some cases, a melatonin receptor agonist, salt thereof, or pharmaceutical composition comprising a melatonin receptor agonist or salt thereof described herein can be administered at a dose of about 0.1, 0.2, 0.3, 0.4, 0.5, 0.6, 0.7, 0.8, 0.9, 1, 1.25, 1.5, 1.75, 2, 2.5, 3, 3.5, 4, 4.5, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 184, 186, 187, 188, 189, 190, 191, 192, 193, 194, 195, 196, 197, 198, 199, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 mg.

TABLE 1
Examples of melatonin receptor agonists

| Name | Properties |
|---|---|
| Circadin ® (Prolonged Release | Slow-release melatonin preparations. Melatonin |
| melatonin 2 mg) | receptor agonist: MT₁ (Kᵢ 0.08 nM) and MT₂ |
| Neurin Pharmaceuticals | (Kᵢ 0.38 nM) |
| Slenyto ® (PEDPRM) | Children/adolescent age-appropriate formulation |
| Neurin Pharmaceuticals | of prolonged-release melatonin. Melatonin |
| | receptor agonist: MT₁ (Kᵢ 0.08 nM) and MT₂ |
| | (Kᵢ 0.38 nM) |
| Ramelteon (Brand name Rozerem) | Melatonin receptor agonist: MT₁ (Kᵢ 0.014 nM) |
| Takeda | and MT₂ Kᵢ 0.11 nM) |
| Tasimelteon (Brand name Hetlioz ®) | Melatonin receptor agonist: MT₁ (Kᵢ 0.30 nM) and |
| Vanda Pharmaceuticals | MT₂ Kᵢ 0.07 nM) |
| Agomelatine (Brand name Valdoxan) | Melatonin receptor agonist (MT₁ (Kᵢ 0.01 nM) and |
| Servier | MT₂ (Kᵢ = 0.12 nM)) and serotonin 5-HT_{2C} |
| | (Kᵢ = 631 nM) and 5-HT_{2B} receptor (Kᵢ = 660 nM) |
| | antagonist |
| TIK-301 (LY-156735) | Melatonin receptor agonist (MT₁ (Kᵢ 81 pM) and |
| Tikvah Pharmaceuticals | MT₂ (Kᵢ = 42 pM)) |
| Piromelatine (Neu-P11) | Melatonin receptor agonist (MT₁ (Kᵢ 22 pM) and |
| Neurim Pharmceuticals | MT₂ (Kᵢ = 34 pM)) |
| N-[2-(5-Chloro-2,6- | Melatonin receptor agonist (MT₁ (Kᵢ = 2.1 nM and |
| dimethoxybenzoimidazol-1- | EC₅₀ = 0.063 nM) and MT₂ (Kᵢ = 0.80 nM and |
| yl)ethyl]acetamide (10b) | EC₅₀ = 0.321 nM))) in CHO cells expressing |
| | human MT1 and MT2 |
| N-[3-(5-Chloro-2-ethoxy-6- | Melatonin receptor agonist (MT₁ (Kᵢ = 13 nM and |
| methoxybenzoimidazol-1- | EC₅₀ = 0.937 nM) and MT₂ (Kᵢ = 1.60 nM and |
| yl)propyl]acetamide (15a) | EC₅₀ = 0.141 nM)) in CHO cells expressing |
| | human MT1 and MT2 |
| N-[2-(2-methoxy-7,8-dihydro-6-oxa-1,3- | Melatonin receptor agonist (MT₁ (EC₅₀ = 0.042 |
| diaza-as-indacen-1-yl)ethyl]acetamide (19a) | nM) and MT₂ (EC₅₀ = 0.049) in CHO cells |
| | expressing human MT1 and MT2 |
| N-[2-(5-methoxy-2- | |
| nitrophenylamino)ethyl]acetamide (3a) | |
| N-[2-(6-methoxybenzoimidazol-1- | |
| y1)ethyl]acetamide (4) | |
| N-[2-(2-amino-5- | |
| methoxyphenylamino)ethyl]acetamide 5a | |
| N-[2-(6-methoxy-2-oxo-2,3- | |
| dihydrobenzoimidazol-1-yl)ethyl]acetamide | |
| (6) | |
| N-[2-(6-methoxy-2- | |
| methylsulfanylbenzoimidazol-1- | |
| yl)ethyl]acetamide (7) | |
| N-[2-(2-ethoxy6-methoxybenzoimidazol-1- | |
| yl)ethyl]acetamide (8a) | |
| N-[2-(2-Ethoxy-6-methoxybenzoimidazol-1- | |
| y1)ethyl propionamide (8b) | |
| N-[2-(2-Ethoxy-6-methoxybenzoimidazol-1- | |
| yl)ethyl]butyramide (8c) | |
| N-[2-(2,6-Dimethoxybenzoimidazol-1- | |
| yl)ethyl]acetamide (8d) | |
| Cyclopropanecarboxylic acid [2-(2-ethoxy- | |
| 6-methoxybenzoimidazol-1-yl)ethyl]amide | |
| (8e) | |
| Cyclobutanecarboxylic acid [2-(2-ethoxy-6- | |
| methoxybenzoimidazol-1-yl)ethyl]amide (8f) | |
| Cyclopentanecarboxylic acid [2-(2-ethoxy- | |
| 6-methoxybenzoimidazol-1-yl)ethyl]amide | |
| (8g) | |
| Cyclohexanecarboxylic acid [2-(2-ethoxy-6- | |
| methoxybenzoimidazol-1-yl)ethyl]amide | |
| (8h) | |
| 4-oxo-4H-pyran-2-carboxylic acid [2-(2- | |
| ethoxy-6-methoxybenzoimidazol-1- | |
| yl)ethyl]amide (8i) | |
| N-[2-(2-Ethoxy-5-methoxybenzoimidazol-1- | |
| yl)ethyl]acetamide (8j) | |
| 2-(2-ethoxy-6-methoxybenzoimidazol-1- | |
| yl)ethylamine (9) | |
| N-[2-(5-chloro-2-ethoxy-6- | |
| methoxybenzoimidazol-1-yl)ethyl]acetamide | |
| (10a) | |
| N-[3-(2-Ethoxy-6-methoxybenzoimidazol-1- | |
| y1)propyl]acetamide (14a) | |
| N-[3-(2,6-Dimethoxybenzoimidazol-1- | |
| y1)propyl]acetamide (14b) | |
| N-[3-(5-Chloro-2,6- | |
| dimethoxybenzoimidazol-1- | |
| yl)propyl]acetamide (15b) | |
| N-[2-(2-Ethoxy-7,8-dihydro-6-oxa-1,3- | |
| diaza-as-indacen-1-y1)ethyl]acetamide (19b) | |

TABLE 2
Kᵢ values of melatonin and melatonin receptors
agonist for MT₁ and MT₂

| | | |
|---|---|---|
| Tasilmelteon | MT₁ (Kᵢ 0.304 nM | MT₂ (Kᵢ 0.0692 nM |
| | [NIH-3T3 cells] and | [NIH-3T3 cells] and |
| | Kᵢ 0.35 nM | Kᵢ 0.17 nM |
| | [CHO-K1 cells]) | [CHO-K1 cells]) |
| Melatonin | human MT₁ | human MT₂ |
| | (Ki 0.102 nM | (Kᵢ 0.096 nM |
| | [CHO-K1 cells]) | [CHO-K1 cells] |
| Ramelteon | human MT₁ | human MT₂ |
| | (Ki 0.118 nM | (Kᵢ 0.115 nM |
| | [CHO-K1 cells]) | [CHO-K1 cells] |
| Agomelatine | human MT₁ | human MT₂ |
| | (Ki 0.102 nM | (Kᵢ 0.112 nM |
| | [CHO-K1 cells]) | [CHO-K1 cells] |

### Pharmacodynamic Profile

In some aspects, the pharmaceutical composition will have a desirable pharmacodynamic (PD) profile within the subject. In some embodiments, pharmaceutical compositions comprise a PD profile within a subject that delivers an effective amount of melatonin receptor agonist to target cells outside of the CNS. In some embodiments, the target cells outside of the CNS comprise cells of the endocrine, reproductive, metabolic, or immune systems, or any combination thereof. In some embodiments, the target cells comprise ovarian cells. In some embodiments, the melatonin receptor agonist functions as a direct antioxidant. In some embodiments, the melatonin receptor agonist directly oxidizes reactive oxygen species (ROS) within target cells, reactive nitrogen species (RNS) within target cells, or a combination thereof. In some embodiments, the melatonin receptor agonist directly oxidizes ROS extracellular to target cells, RNS extracellular to target cells, or a combination thereof. In some embodiments, the melatonin receptor agonist directly oxidizes intracellular ROS, intracellular RNS, or a combination thereof within target cells. In some embodiments, the melatonin receptor agonist directly oxidizes ROS, RNS, or a combination thereof within target cell mitochondria. In some embodiments, the melatonin receptor agonist functions to locally upregulate melatonin receptor signal transduction. In some embodiments, the local upregulation of melatonin receptor signal transduction occurs in target cells comprising ovarian cells, oocytes, granulosa cells, theca cells, ovarian stromal cells, ovarian epithelial cells, Fallopian tube cells, uterine cells, endometrial cells, myometrial cells, cervical cells, adrenal gland cells, pancreatic cells, thyroid gland cells, parathyroid gland cells, skeletal muscle cells, liver cells, white adipose tissue cells, brown adipose tissue cells, osteoblasts, osteoclasts, endothelial cells, hair follicle cells, lymphocytes, neutrophils, monocytes, or macrophages, or progenitors of any of the above cells types. In some embodiments, the local upregulation of melatonin receptor signal transduction increases expression of a plurality of cellular antioxidants. In some embodiments, the plurality of cellular antioxidants comprises superoxide dismutase, glutathione reductase, glutathione peroxidase, or catalase, or any combination thereof. In some embodiments, the plurality of cellular antioxidants comprise the genes SOD1, SOD2, GSR, GPX1, GPX2, GPX3, GPX4, GPX5, GPX6, GPX7, GPX8, or CAT. In some embodiments, the local upregulation of melatonin receptor signal transduction increases activity of a plurality of cellular antioxidants. In some embodiments, the plurality of cellular antioxidants comprises superoxide dismutase, glutathione reductase, glutathione peroxidase, or catalase, or any combination thereof. In some embodiments, the local upregulation of melatonin receptor signal transduction results in a change in intracellular cyclic nucleotides in a plurality of target cells. In some embodiments, the intracellular cyclic nucleotides comprise cAMP, cGMP, inositol 1,4,5-trisphosphate (IP₃), or diacylglyerol (DAG), or any combination thereof. In some embodiments, IP₃ and DAG serve as messengers for activation of the PI3K/AKT pathway. In some embodiments, the local upregulation of melatonin receptor signal transduction results in a change in cytosolic calcium levels in a plurality of target cells. In some embodiments, the local upregulation of melatonin receptor signal transduction results in a change in mitochondrial calcium levels in a plurality of target cells. In some embodiments, the local upregulation of melatonin receptor signal transduction results in activation of protein kinase C (PKC) subtypes in a plurality of target cells. In some embodiments, the PKC subtypes comprise conventional, novel, or atypical PKC subtypes. In some embodiments, the local upregulation of melatonin receptor signal transduction results in changes in intracellular localization of steroid hormone receptors. In some embodiments, the local upregulation of melatonin receptor signal transduction results in changes in expression of gonadotrophin-releasing hormone receptors, luteinizing hormone receptors and/or follicle-stimulating hormone receptors. In some embodiments, the changes in expression of hormone receptors comprises increases in expression level of mRNAs coding for hormone receptors. In some embodiments, the local upregulation of melatonin receptor signal transduction results in a change in secretion of progesterone from granulosa cells. In some embodiments, the change in secretion of progesterone comprises elevated secretion of progesterone. In some embodiments, the local upregulation of melatonin receptor signal transduction results in alterations in secretion of a plurality of endocrine hormones comprising insulin, glucagon, somatostatin, pancreatic polypeptide, cholecystokinin, secretin, amylin, gastrin, or thyroxine, or any combination thereof. In some embodiments, the local upregulation of melatonin receptor signal transduction results in activation of MAPK pathway signal transduction. In some embodiments, the MAPK pathway signal transduction pathway activated is the MAPK-JNK/P38 signaling pathway. In some embodiments, the local upregulation of melatonin receptor signal transduction results in activation of a G-protein signaling pathway in a plurality of target cells. In some embodiments, the local upregulation of melatonin receptor signal transduction results in inhibition of adenyl cyclase in a plurality of target cells. In some embodiments, the local upregulation of melatonin receptor signal transduction results in activation of phospholipase C in a plurality of target cells. In some embodiments, the local upregulation of melatonin receptor signal transduction results in a change in lipid metabolism in a plurality of target cells. In some embodiments, the local upregulation of melatonin receptor signal transduction results in a change in follicular development. In some embodiments, the change in follicular development comprises prevention of granulosa cell apoptosis in a plurality of granulosa cells. In some embodiments, the prevention of granulosa cell apoptosis improves follicular development. In some embodiments, the local upregulation of melatonin receptor signal transduction results in improved oocyte quality. In some embodiments, the local upregulation of melatonin receptor signal transduction improves folliculogenesis and/or ovulation.

### Melatonin Receptor Agonist Formulation

Described herein are pharmaceutical compositions formulated to deliver a therapeutically effective amount of a melatonin receptor agonist to tissues in the endocrine, reproductive, metabolic, or immune systems, or in any combination thereof. In some embodiments, the melatonin receptor agonist may be formulated as a gel. In some embodiments, the melatonin receptor agonist may be formulated as a cream. In some embodiments, the melatonin receptor agonist may be formulated as an ointment. In some embodiments, the melatonin receptor agonist may be formulated as a solution. In some embodiments, the melatonin receptor agonist may be formulated as a powder. In some embodiments, the melatonin receptor agonist may be formulated as a paste. In some embodiments, the melatonin receptor agonist may be formulated as a foam. In some embodiments, the melatonin receptor agonist may be formulated as an emulsion. In some embodiments, the melatonin receptor agonist may be formulated as a lotion. In some embodiments, the formulation may comprise polyethylene glycol (PEG). In some embodiments, the melatonin receptor agonist may be PEGylated. In some embodiments, the melatonin receptor agonist may be formulated in a hydrogel. In some embodiments, the melatonin receptor agonist formulation comprises a liposome. In some embodiments, melatonin receptor agonists loaded into liposomes leads to a superior PK profile due to poor solubility of melatonin receptor agonists in aqueous solution. In some embodiments, the melatonin receptor agonist formulation comprises a nanoparticle. In some embodiments, the melatonin receptor agonist formulation comprises a nanosuspension. In some embodiments, the melatonin receptor agonist formulation comprises a nanoemulsion. In some embodiments, the nanoparticle comprises a magnetic nanoparticle, a zinc oxide nanoparticle, a selenium-coated nanoparticle, a solid lipid nanoparticle, a nanostructured lipid carrier, a chitosan-coated nanoparticle, polymeric micelles, a cyclodextrin, or a dendrimer. In some embodiments, the formulation comprises a delayed-release, a sustained-release, an extended-release, a prolonged-release, or a slow-release formulation. In some embodiments, the melatonin receptor agonist is at least partially encapsulated in a liposome, a magnetic nanoparticle, a zinc oxide nanoparticle, a selenium-coated nanoparticle, a solid lipid nanoparticle, a nanostructured lipid carrier, a chitosan-coated nanoparticle, polymeric micelles, a cyclodextrin, or a dendrimer. In some embodiments, the formulation protects the melatonin receptor agonist from premature oxidation. In some embodiments, the protection from premature oxidation increases the potency of the pharmaceutical composition due to a greater retention of direct antioxidant and signaling properties of non-oxidized melatonin receptor agonists. In some embodiments, the formulation delivers higher melatonin receptor agonist exposure following local delivery into the reproductive tract. In some embodiments, the formulation comprises a slower release of melatonin receptor agonist than a conventional oral formulation. In some embodiments, the formulation comprises a slower release of melatonin receptor agonist than a conventional liquid formulation. In some embodiments, the melatonin receptor agonist may be formulated as a matrix. In some embodiments, the matrix comprises a bioresorbable polymer. In some embodiments, the bioresorbable polymer is poly (lactic-co-glycolic acid) (PLGA). In some embodiments, the bioresorbable polymer is poly (ethylene glycol) (PEG). In some embodiments, the bioresorbable polymer is poly (vinyl alcohol) (PVA). In some embodiments, the bioresorbable polymer is poly (glycolic acid) (PGA). In some embodiments, the bioresorbable polymer is poly[2-(dimethylamino)ethyl methacrylate] (DMAEM). In some embodiments, the matrix comprises a bioinert polymer. In some embodiments, the bioinert polymer is ethylene vinyl acetate. In some embodiments, the bioinert polymer is cellulose acetate. In some embodiments, the bioinert polymer is low density polyethylene (LDPE). In some embodiments, the matrix is a solid ethylene vinyl acetate polymer matrix. In some embodiments, the matrix is a mixture of starch with a solid ethylene vinyl acetate polymer. In some embodiments, the matrix is a mixture of starch with cellulose acetate. In some embodiments, the matrix is a mixture of starch with LDPE. In some embodiments, the matrix is a mixture of starch with ethylene vinyl alcohol copolymer. In some embodiments the matrix may release one active ingredient comprising a melatonin receptor agonist. In some embodiments, the matrix may release two or more active ingredients comprising a melatonin receptor agonist. In some embodiments, the matrix may release two or more active ingredients comprising a plurality of melatonin receptor agonists. In some embodiments, the matrix may allow for a release of the melatonin receptor agonist without the need for a membrane to contain the melatonin receptor agonist. In some embodiments, the matrix may allow for a release of the melatonin receptor agonist without the need for a reservoir to contain the melatonin receptor agonist. In some embodiments, the matrix may allow for a delayed release of the melatonin receptor agonist. In some embodiments, the matrix may allow for a sustained release of the melatonin receptor agonist. In some embodiments, the matrix may allow for an extended release of the melatonin receptor agonist. In some embodiments, the matrix may allow for a prolonged release of the melatonin receptor agonist. In some embodiments, the matrix may allow for a slow release of the melatonin receptor agonist. In some embodiments, the matrix is contained within and formulated for administration by a drug delivery device. In some embodiments, the matrix is contained within and formulated for administration by a vaginal ring. In some embodiments, the matrix is contained within and formulated for administration by a vaginal tablet. In some embodiments, the matrix is contained within and formulated for administration by a pessary. In some embodiments, the matrix is contained within and formulated for administration by a suppository. In some embodiments, the matrix is contained within and formulated for administration by a patch. In some embodiments, the matrix is contained within and formulated for administration by an intrauterine device.

Described herein are pharmaceutical compositions formulated to deliver a therapeutically effective amount of a melatonin receptor agonist to peripheral target tissues that have reduced capacity to accumulate in the CNS. In some embodiments, the pharmaceutical compositions are formulated for parenteral administration (e.g., oral delivery, IV administration, subcutaneous administration, etc.). In some embodiments, the formulation comprises a modification that lowers lipophilicity. In some embodiments, lowered lipophilicity results in less cell penetration of melatonin receptor agonist compared to an equivalent amount of melatonin. In some embodiments, lowered lipophilicity results in decreased local tissue diffusion of melatonin receptor agonist compared to an equivalent amount of melatonin. In some embodiments, lowered lipophilicity results in less active transport across the BBB of melatonin receptor agonist compared to an equivalent amount of melatonin. In some embodiments, lowered lipophilicity results in increased expulsion from the CSF via BBB egress transporters of melatonin receptor agonist compared to an equivalent amount of melatonin. In some embodiments, lowered lipophilicity results in increased metabolism within the CNS of melatonin receptor agonist compared to an equivalent amount of melatonin. In some embodiments, lowered lipophilicity results in increased CNS clearance of melatonin receptor agonist compared to an equivalent amount of melatonin. In some embodiments, lowered lipophilicity results in diminished permeability across the BBB of melatonin receptor agonist compared to an equivalent amount of melatonin. In some embodiments, the formulation comprises a modification that lowers hydrophilicity. In some embodiments, lowered hydrophilicity results in less cell penetration of melatonin receptor agonist compared to an equivalent amount of melatonin. In some embodiments, lowered hydrophilicity results in decreased local tissue diffusion of melatonin receptor agonist compared to an equivalent amount of melatonin. In some embodiments, lowered hydrophilicity results in less active transport across the BBB of melatonin receptor agonist compared to an equivalent amount of melatonin. In some embodiments, lowered hydrophilicity results in increased expulsion from the CSF via BBB egress transporters of melatonin receptor agonist compared to an equivalent amount of melatonin. In some embodiments, lowered hydrophilicity results in increased metabolism within the CNS of melatonin receptor agonist compared to an equivalent amount of melatonin. In some embodiments, lowered hydrophilicity results in increased CNS clearance of melatonin receptor agonist compared to an equivalent amount of melatonin. In some embodiments, lowered hydrophilicity results in diminished permeability across the BBB of melatonin receptor agonist compared to an equivalent amount of melatonin. In some embodiments, less than about 50%, 45%, 40%, 35%, 30%, 25%, 22%, 20%, 18%, 15%, 13%, 10%, 9%, 8%, 7%, 6%, 5%, 4.5%, 4%, 3.5%, 3%, 2.5%, 2%, 1.5%, 1%, 0.75%, 0.5%, 0.3%, 0.2%, 0.1%, 0.05%, 0.02%, 0.01%, 0.005%, or 0.001% of the melatonin receptor agonist contained within the pharmaceutical composition penetrates the blood-brain barrier of the subject.

In some aspects, when comparing an amount of a melatonin receptor agonist to an equivalent amount of melatonin, a comparison is made by comparing amounts of melatonin receptor agonist and melatonin each at about the same molarity in a sample. In some embodiments, the comparison is made by comparing amounts of Circadin® and melatonin each at about the same molarity in a sample. In some embodiments, the comparison is made by comparing amounts of Slenyto® and melatonin each at about the same molarity in a sample. In some embodiments, the comparison is made by comparing amounts of Ramelteon and melatonin each at about the same molarity in a sample. In some embodiments, the comparison is made by comparing amounts of Tasimelteon and melatonin each at about the same molarity in a sample. In some embodiments, the comparison is made by comparing amounts of Agomelatine and melatonin each at about the same molarity in a sample. In some embodiments, the comparison is made by comparing amounts of TIK-301 and melatonin each at about the same molarity in a sample. In some embodiments, the comparison is made by comparing amounts of Piromelatine and melatonin each at about the same molarity in a sample. In some embodiments, the comparison is made by comparing amounts of N-[2-(5-Chloro-2,6-dimethoxybenzoimidazol-1-yl)ethyl]acetamide and melatonin each at about the same molarity in a sample. In some embodiments, the comparison is made by comparing amounts of N-[3-(5-Chloro-2-ethoxy-6-methoxybenzoimidazol-1-yl) propyl]acetamide and melatonin each at about the same molarity in a sample. In some embodiments, the comparison is made by comparing amounts of N-[2-(2-methoxy-7,8-dihydro-6-oxa-1,3-diaza-as-indacen-1-yl)ethyl]acetamide and melatonin each at about the same molarity in a sample.

In some aspects, the pharmaceutical composition comprises a melatonin receptor agonist conjugated to another molecule thereby reducing CNS accumulation of melatonin receptor agonist compared to an equivalent amount of melatonin. In some embodiments, the formulation comprises a modification that reduces the capacity of the melatonin receptor agonist-conjugate to cross the blood-brain barrier. In some embodiments, the conjugate comprises a hydrophilic molecule, a carbohydrate molecule, a peptide, or a synthetic molecule. In some embodiments, the formulation comprises a modification that prevents active transport of the agonist through the BBB. In some embodiments, the formulation comprises a modification that increases egress of the melatonin receptor agonist outside of the CNS. In some embodiments, the formulation comprises a modification that accelerates the metabolism and/or clearance of the melatonin receptor agonist in the CNS. In some embodiments, the formulation comprises a modification that increases binding of the melatonin receptor agonist to a plurality of protein that decrease its free concentration in the CNS. In some embodiments, the reduced capacity of the melatonin receptor agonist to accumulate in the CNS allows for a broader therapeutic dosage range for target cells in peripheral tissues. In some embodiments, the broader therapeutic dosage range for target cells in peripheral tissues does not substantially impact or impair CNS function. In some embodiments, the broader therapeutic dosage range for target cells in peripheral tissues has limited impact on CNS function. In some embodiments, the broader therapeutic dosage range for target cells in peripheral tissues may mitigate potential side effects in the CNS such as somnolence and defects in sleep patterns, circadian rhythms, motor control, cognitive functions, or memory extinction. In some embodiments, the reduced capacity of the melatonin receptor agonist to accumulate in the CNS may allow for a higher systemic concentration of melatonin receptor agonist while maintaining a favorable safety profile. In some embodiments, the reduced capacity of the melatonin receptor agonist to accumulate in the CNS may allow for delivery by intravenous injection. In some embodiments, the reduced capacity of the melatonin receptor agonist to accumulate in the CNS may allow for delivery by oral administration. In some embodiments, the reduced capacity of the melatonin receptor agonist to accumulate in the CNS may allow for delivery by a drug delivery device, such as a vaginal ring, a vaginal tablet, a pessary, a suppository, a patch, or a intrauterine device.

Described herein are types of administration of pharmaceutical compositions formulated to deliver a therapeutically effective amount of a melatonin receptor agonist to tissues in the endocrine, reproductive, metabolic, vascular, skeletal, integumentary or immune systems, or in any combination thereof. In some aspects, the pharmaceutical composition is delivered locally near a target tissue. In some embodiments, the target tissue comprises cervical tissue, myometrial tissue, or oviductal tissue, or any combination thereof. In some embodiments, the local delivery comprises local injection. In some embodiments, the local delivery comprises transdermal or subcutaneous delivery. In some embodiments, the local delivery comprises delivery through a drug delivery device. In some embodiments, the local delivery comprises delivery through a vaginal ring, a vaginal gel, a vaginal film, a vaginal tablet, a pessary, a suppository, or a patch. In some embodiments, the local delivery comprises intrauterine or intravaginal delivery. In some embodiments, the local delivery results in a higher concentration of melatonin receptor agonist near the site of delivery than at a peripheral location farther away from the site of local delivery. In some embodiments, local delivery comprises delivery by a device. In some embodiments, the device comprises an intrauterine device. In some embodiments, delivery by a device that allows time-controlled release of the pharmaceutical composition. In some embodiments, time-controlled release comprises remote-controlled release. In some embodiments, time-controlled release permits release from an inserted chronic device that delivers the pharmaceutical composition at a desired frequency of administration. In some embodiments, the desired frequency of administration is a diurnal frequency. In some embodiments, the desired frequency of administration is a nocturnal frequency. In some embodiments, the composition is administered daily before bedtime. In some embodiments, the desired frequency of administration avoids somnolence effects of the melatonin receptor agonist. In some embodiments, the pharmaceutical composition is delivered to result in continuous exposure of the melatonin receptor agonists to a plurality of target cells. In some embodiments, a desired pattern of continuous or diurnal daily exposure to melatonin receptor agonist to address multiple phenotypes of PCOS may differ among indications.

In some aspects, melatonin receptor agonists delivered by vaginal gels or by drug delivery device (e.g., vaginal rings or intrauterine devices) can achieve superior control of the symptoms of PCOS that derive from the endocrine, inflammatory and oxidative stress pathways and avoid the sleep-inducing effects that accompany orally administered melatonin agonists that achieve high concentrations in the CNS.

In some aspects, pharmaceutical compositions described herein are administered to a subject in need thereof according to a treatment regimen. In some embodiments, the treatment regimen comprises a single administration. In some embodiments, the treatment regimen comprises at least one administration. In some embodiments, the treatment regimen comprises 1, 2, 3, 4, 5, 6, 7, 8, 9, 10, 11, 12, 13, 14, 15, 16, 17, 18, 19, 20 or more administrations. In some embodiments, an administration comprises delivery of an effective amount of melatonin receptor agonist to target cells. In some embodiments, the effective amount contained within one administration is an effective amount for at least 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 35, 42, 49, 56, 60, 61, 62, or 90 days. In some embodiments, one administration comprises an effective amount for at least 1 week, at least 2 weeks, at least 4 week, at least 2 months, or at least 6 months.

### Uses of Pharmaceutical Compositions

In some aspects, pharmaceutical compositions described herein may be used to modulate a phenotype affected by melatonin in a subject. In some embodiments, the phenotype affected by melatonin is caused by melatonin system dysfunction. In some embodiments, the phenotype affected by melatonin comprises a symptom that can be alleviated or treated by increasing local melatonin concentration or activation of local melatonin receptors. In some embodiments, the phenotype affected by melatonin comprises a symptom that can be alleviated or treated by activating the melatonin receptor mediated signaling pathway. In some embodiments, the phenotype affected by melatonin is susceptible to age-related changes in melatonin distribution. In some embodiments, age-related changes in melatonin distribution comprise decreased melatonin production. In some embodiments, age-related changes in melatonin distribution comprise low melatonin levels in some peripheral tissues and normal or high plasma concentrations of melatonin. In some embodiments, age-related changes in melatonin distribution comprise decreased extrapineal melatonin production. In some embodiments, age-related changes in melatonin distribution comprise decreased ovarian melatonin production.

In some aspects, tissue damage as a result of disease or other insult results in a phenotype affected by melatonin in the subject. In some embodiments, administration of a pharmaceutical composition comprising a melatonin receptor agonist in a subject with a phenotype affected by melatonin results in a slowdown of disease progression. In some embodiments, administration of a pharmaceutical composition comprising a melatonin receptor agonist in a subject with a phenotype affected by melatonin results in a halting of disease progression. In some embodiments, administration of a pharmaceutical composition comprising a melatonin receptor agonist in a subject with a phenotype affected by melatonin results in an improvement in an aspect of a disease. In some embodiments, administration of a pharmaceutical composition comprising a melatonin receptor agonist in a subject with a phenotype affected by melatonin results in alleviation or reduction of intensity or frequency of a symptom of a disease (e.g., pain, infertility, hyperandrogenism, endocrine unbalance, ovulatory dysfunction, weight gain, insulin insensitivity, glucose intolerance, bone density loss, hair loss, or hypertension). In some embodiments, the tissue damage develops following administration of a chemotherapy to the subject. In some embodiments, the phenotype caused by the tissue damage comprises chemotherapy-induced ovarian dysfunction (CIOD). In some embodiments, the phenotype caused by the tissue damage comprises chemotherapy-induced ovarian failure (CIOF). In some embodiments, CIOD or CIOF are induced in the subject by exposure to one or more chemotherapy treatments. In some aspects, CIOD comprises disruption of ovarian reproductive function. In some aspects, CIOD comprises disruption of ovarian endocrine function. In some aspects, CIOD comprises disruption of ovarian reproductive and endocrine function. In some aspects, CIOF comprises disruption of ovarian reproductive and endocrine function. Examples of chemotherapy treatment agents that can induce ovarian tissue damage in the subject include altretamine, bendamustine, busulfan, barboplatin, barmustine, chlorambucil, cisplatin, cyclophosphamide, dacarbazine, doxorubicin, ifosfamide, lomustine, mechlorethamine, melphalan, oxaliplatin, temozolomide, thiotepa, and trabectedin. In some instances, the chemotherapy treatment agent is an alkylating agent. In some instances, the chemotherapy treatment agent causes premature ovarian insufficiency by inducing death and/or accelerated activation of primordial follicles and increased atresia of growing follicles. In some instances, the subject has received one or more chemotherapy treatments comprising administration of one or more chemotherapy treatment agents. In some instances, the subject is receiving a chemotherapy treatment comprising administration of one or more chemotherapy treatment agents. In some instances, the subject will receive a chemotherapy treatment comprising administration of one or more chemotherapy treatment agents. In some aspects, the use of pharmaceutical compositions described herein comprises administering the pharmaceutical composition comprising a melatonin receptor agonist to the subject following one or more chemotherapy treatments in the subject. In some aspects, the use of pharmaceutical compositions described herein comprises administering the pharmaceutical composition comprising a melatonin receptor agonist to the subject concurrently with one or more chemotherapy treatments in the subject. In some aspects, the use of pharmaceutical compositions described herein comprises administering the pharmaceutical composition comprising a melatonin receptor agonist to the subject prior to one or more chemotherapy treatments in the subject. In some embodiments, use of the pharmaceutical composition comprising a melatonin receptor agonist attenuates cyclophosphamide-induced primordial follicle loss. In some embodiments, use of the pharmaceutical composition comprising a melatonin receptor agonist attenuates cisplatin-induced primordial follicle loss. In some embodiments, use of the pharmaceutical composition comprising a melatonin receptor agonist attenuates doxorubicin-induced primordial follicle loss. In some embodiments, pretreatment with the pharmaceutical composition comprising a melatonin receptor agonist prevents primordial follicle loss due to CIOD. In some embodiments, pretreatment with the pharmaceutical composition comprising a melatonin receptor agonist attenuates primordial follicle loss due to CIOD. In some embodiments, pretreatment with the pharmaceutical composition comprising a melatonin receptor agonist decreases growing follicle atresia due to CIOD. In some embodiments, pretreatment with the pharmaceutical composition comprising a melatonin receptor agonist decreases ovarian mitochondrial damage due to CIOD. In some embodiments, pretreatment with the pharmaceutical composition comprising a melatonin receptor agonist reduces ovarian apoptosis following administration of cyclophosphamide. In some embodiments, pretreatment with the pharmaceutical composition comprising a melatonin receptor agonist reduces ovarian oxidative damage following administration of cyclophosphamide. In some embodiments, pretreatment with the pharmaceutical composition comprising a melatonin receptor agonist preserves ovarian hormonal levels following administration of cyclophosphamide. In some embodiments, pretreatment with the pharmaceutical composition comprising a melatonin receptor agonist improves follicular morphology following administration of cyclophosphamide. In some embodiments, pretreatment with the pharmaceutical composition comprising a melatonin receptor agonist preserves granulosa cell proliferation following administration of cyclophosphamide. In some embodiments, pretreatment with the pharmaceutical composition comprising a melatonin receptor agonist prevents ovarian reserve decline following administration of cisplatin. In some embodiments, pretreatment with the pharmaceutical composition comprising a melatonin receptor agonist maintains a threshold level of AMH and BMP15 ovarian expression following administration of cisplatin. In some embodiments, pretreatment with the pharmaceutical composition comprising a melatonin receptor agonist inhibits ovarian inflammation following administration of cisplatin. In some embodiments, cisplatin-induced ovarian inflammation is measured by assaying ovarian expression levels of IL-1β and/or IL-18. In some embodiments, pretreatment with the pharmaceutical composition comprising a melatonin receptor agonist protects an ovary of the subject from cisplatin-induced mitochondrial damage following administration of cisplatin to the subject. In some embodiments, pretreatment with the pharmaceutical composition comprising a melatonin receptor agonist decreases levels of reactive oxygen species following administration of cisplatin. In some embodiments, pretreatment with the pharmaceutical composition comprising a melatonin receptor agonist increases antioxidant enzyme activity following administration of cisplatin. In some embodiments, pretreatment with the pharmaceutical composition comprising a melatonin receptor agonist inhibits the development of mitochondrial oxidative stress following administration of cisplatin. In some embodiments, pretreatment with the pharmaceutical composition comprising a melatonin receptor agonist inhibits ovarian apoptosis administration of cisplatin. In some embodiments, pretreatment with the pharmaceutical composition comprising a melatonin receptor agonist reduces an extent of serum estradiol (E2) and AMH decrease following administration of cisplatin to the subject. In some embodiments, pretreatment with the pharmaceutical composition comprising a melatonin receptor agonist reduces an extent of serum LH and FSH increase following administration of cisplatin to the subject. In some embodiments, pretreatment with the pharmaceutical composition comprising a melatonin receptor agonist reduces damage to the follicles and corpus luteum following administration of cisplatin to the subject. In some embodiments, pretreatment with the pharmaceutical composition comprising a melatonin receptor agonist reduces inflammatory infiltrates in the ovary following administration of cisplatin to the subject. In some embodiments, pretreatment with the pharmaceutical composition comprising a melatonin receptor agonist mitigates an extent of increased ovarian collagen deposition following administration of cisplatin to the subject. In some embodiments, pretreatment with the pharmaceutical composition comprising a melatonin receptor agonist prevent ovarian fibrosis following administration of cisplatin to the subject. In some embodiments, pretreatment with the pharmaceutical composition comprising a melatonin receptor agonist reduces apoptosis in granulosa and theca cells, stroma, and oocytes following administration of cisplatin to the subject.

In some aspects, the phenotype affected by melatonin in the subject comprises impairment in endocrine cells, in reproductive cells, in metabolic cells, in vascular cells, in skeletal cells, in integumentary cells or in immune cells, or any combination thereof. In some embodiments, the phenotype affected by melatonin in the subject responds to therapy comprising administration of a melatonin receptor agonist. In some embodiments, the phenotype in the subject that responds to melatonin receptor agonist therapy comprises an ovarian phenotype, an endometrial phenotype, an inflammatory phenotype, a metabolic phenotype, a vascular phenotype, a skeletal phenotype, an integumentary phenotype or a liver phenotype. In some embodiments, the ovarian phenotype comprises abnormal or reduced folliculogenesis, altered ovarian progesterone production, altered luteal phase production of progesterone, or poor oocyte maturation. In some embodiments, the endometrial phenotype comprises endometriosis, endometriosis-associated ovarian oxidative stress, or adenomyosis. In some embodiments, the vascular phenotype comprises abnormal blood pressure or hypertension. In some embodiments, the skeletal phenotype comprises bone density loss. In some embodiments, the integumentary phenotype comprises hair loss. In some embodiments, the inflammatory phenotype comprises low grade systemic inflammatory, local idiopathic inflammation, salpingitis, abnormal immune response, autoimmune damage, or poor embryo development. In some embodiments, the metabolic phenotype comprises elevated insulin resistance, dysregulated glucose homeostasis, excess weight or excess body mass index (BMI), or increased oxidative stress.

In some embodiments, the phenotype in the subject that responds to melatonin receptor agonist therapy comprises an aspect or a symptom of a particular disease indication or condition. In some embodiments, the aspect of a particular disease indication or condition that responds to melatonin receptor agonist therapy comprises ovulatory dysfunction, luteal insufficiency, PCOS, anovulatory PCOS, insulin resistance in PCOS, glucose tolerance in PCOS, risk of nonalcoholic fatty liver disease (NAFLD) in PCOS patients, weight control in PCOS, uterine inflammatory diseases, inflammation in PCOS, hyperandrogenism, hirsutism, diminished uterine receptivity, an ART outcome, pre-eclampsia, endometriosis, endometriosis-associated infertility, and endometriosis-associated chronic pelvic pain (EACPP), menopause-related bone density loss, menopause-related hypertension, menopause-related hair loss, menopause-related weight gain, menopause-related glucose homeostasis disruption. In some embodiments, the aspect of a particular disease indication or condition that responds to melatonin receptor agonist therapy comprises chemotherapy-induced ovarian dysfunction (CIOD).

In some aspects, use of pharmaceutical compositions described herein may yield an improved ART outcome. In some embodiments, the improved ART outcome comprises favorable embryo implantation and continuation of pregnancy, higher egg quality, higher embryo quality, a well-vascularized uterus, a quiescent uterus permitting adequate transport of blood at normal blood pressures to the placenta and developing embryo, or improved ovarian health. In some embodiments, improved ovarian health is measured by ovarian follicular reserve, ovarian follicular growth and maturation, and regular frequency of ovulation, oocyte quality and viability. In some embodiments, use of pharmaceutical compositions described herein may allow women taking a melatonin receptor agonist for treatment of the symptoms of PCOS, endometriosis, menopause, inflammation of a reproductive tract, or CIOD to conceive while on therapy. Other methods of treatment use oral contraceptive, clomiphene, or GnRH analog treatments in PCOS, endometriosis, menopause, inflammation of a reproductive tract, or CIOD and may not allow women to conceive while on therapy. In some embodiments, use of pharmaceutical compositions described herein may allow women to maintain regular ovarian and menstrual cycles with prolonged treatment cycles of 1 year or 2 years. In some embodiments, use of pharmaceutical compositions described herein may lead to improved glucose utilization which can in turn benefit fertility and conception.

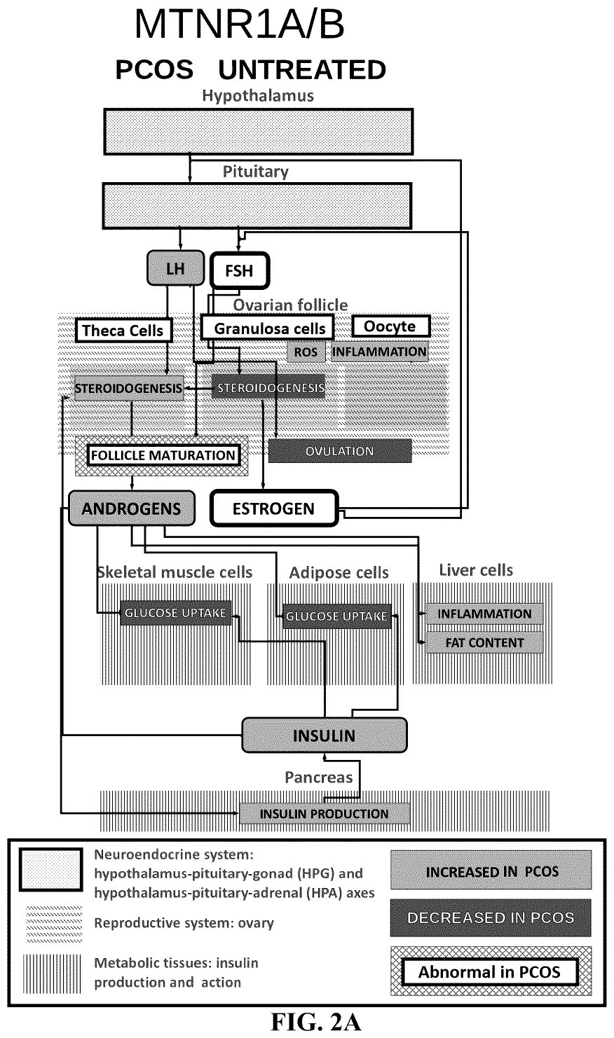

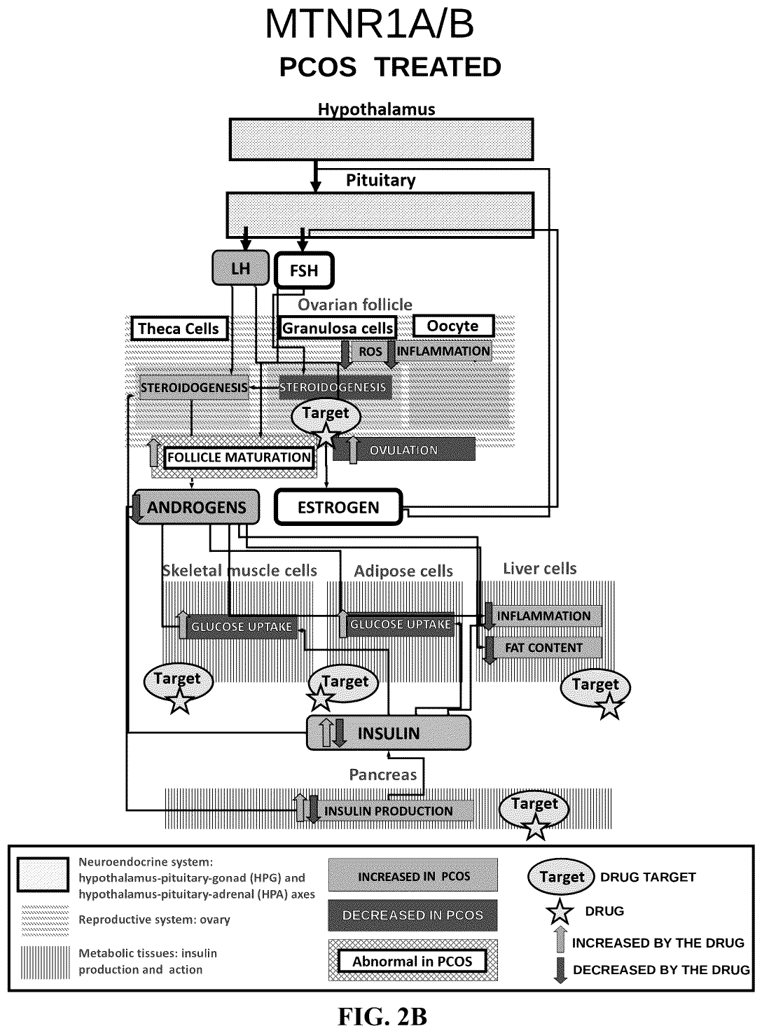

FIG. 2 illustrates a mode of action for a melatonin receptor agonist in PCOS. FIG. 2A lists in an untreated PCOS subject, a diagram linking biological systems, cell types, biological processes, and hormones that interact in PCOS pathology and symptomatology and are either increased, decreased, or abnormal in PCOS. FIG. 2B lists in a melatonin receptor agonist-treated PCOS subject, a diagram linking biological systems, cell types, biological processes, and hormones that interact in PCOS pathology and symptomatology, points of drug target sites, and changes elicited by drug treatment. Melatonin receptor agonist-treatment can have a positive effect on oocyte and embryo maturation and quality. Melatonin receptor agonist-treatment can yield a more favorable ART outcome. This favorable ART outcome can be due to both increased direct ROS scavenging action of melatonin receptor agonist in target tissue and increased melatonin receptor signaling mediated by melatonin receptor agonist in target tissue. The increased melatonin receptor signaling can produce anti-inflammatory and antioxidant effects in ovarian follicles that impact a favorable ART outcome and oocyte and embryo maturation and quality. The increased melatonin receptor signaling can have an anti-apoptotic effect on ovarian granulosa cells which impacts a favorable ART outcome and oocyte and embryo maturation and quality. The increased melatonin receptor signaling can help restore reproductive function by effective steroidogenesis (e.g., controlling progesterone production or decreasing androgen levels). This regulation of androgen levels indicates that melatonin receptor agonist treatment can improve hyperandrogenism, or hirsutism, or both in a subject with PCOS. The increased melatonin receptor signaling can lead to improved glucose tolerance in a subject receiving treatment. The increased melatonin receptor signaling can increase glucose update in adipose and muscle cells. In some subjects with PCOS, increased insulin resistance could be effectively treated by increasing melatonin receptor signaling in target cells. In some subjects with PCOS, increasing melatonin receptor signaling can yield increased insulin secretion from pancreatic beta cells. In some subjects with PCOS, increasing melatonin receptor signaling can yield an improvement in symptoms of non-alcoholic fatty liver disease (NAFLD). In some subjects with PCOS, increasing melatonin receptor signaling can protect the subject from developing NAFLD. In some embodiments, increasing melatonin receptor signaling can protect a subject from liver dysfunction. In some embodiments, increasing melatonin receptor signaling can improve glucose metabolism in a subject.

### Patient Selection and Stratification

In some aspects, a subject with a genetic condition disrupting the melatonin pathway can benefit from treatment including administration of a melatonin receptor agonist. In some embodiments, the subject with a genetic condition disrupting the melatonin pathway can comprise a mutation or an occurrence of an allele with a minor population frequency in MTNR1A or MTNR1B. In some embodiments, a subject with no known genetic conditions disrupting the melatonin pathway can benefit from treatment including administration of a melatonin receptor agonist.

PCOS patients or patients with a metabolic condition affecting reproduction can benefit from a treatment that includes administration of a melatonin receptor agonist. Clinical studies have demonstrated a beneficial effect of melatonin on hyperandrogenism, inflammation, ART outcome, and metabolic function in PCOS patients or patients with metabolic disturbances. Therefore, selection of PCOS patients with these characteristics or risks for these characteristics can be considered in determination of use of a treatment comprising administration of a melatonin receptor agonist.

PCOS patients or patients with a metabolic condition affecting reproduction can benefit from a treatment that includes administration of a melatonin receptor agonist. Clinical studies have demonstrated a beneficial effect of melatonin on hyperandrogenism, inflammation, ART outcome, and metabolic function in PCOS patients or patients with metabolic disturbances. Therefore, selection of PCOS patients with these characteristics or risks for these characteristics can be considered in determination of use of a treatment comprising administration of a melatonin receptor agonist.

Endometriosis patients can benefit from a treatment that includes administration of a melatonin receptor agonist. Clinical studies have demonstrated a beneficial effect of melatonin on pelvic pain in endometriosis patients. Preclinical studies have demonstrated that melatonin promotes endometriosis lesion regression in animals. Therefore, in some embodiments, selection of endometriosis patients with these characteristics or risks for developing these characteristics can be considered in determination of use of a treatment comprising administration of a melatonin receptor agonist.

Perimenopausal and postmenopausal women can benefit from a treatment that includes administration of a melatonin receptor agonist. In some embodiments, there is a beneficial effect of melatonin on menopause-associated symptoms such as bone density loss, weight gain, blood pressure regulation, hair loss and inflammation. Therefore, in some embodiments, selection of perimenopausal and postmenopausal women with these characteristics or risks for developing these characteristics can be considered in determination of use of a treatment comprising administration of a melatonin receptor agonist.

Women about to undergo, currently undergoing, or who have previously undergone a chemotherapy treatment can benefit from a treatment that includes administration of a melatonin receptor agonist. In some embodiments, there is a beneficial effect of melatonin on CIOD symptoms such as hot flashes, osteoporosis, risk of infertility, infertility, sleep disturbances, joint pain, anxiety, depression, sexual dysfunction, increased risk of cardiovascular disease, loss of primordial ovarian follicles, accelerated activation of primordial ovarian follicles, follicular atresia, stromal tissue damage, ovarian vascular damage, ovarian inflammation, reduction in ovarian reserve, increase in rate of age-dependent loss of ovarian reserve, induction of fibrosis, early menopause, premature ovarian insufficiency, premature ovarian failure, induction of apoptosis in mature ovarian follicles, decreased estrogen production, decreased anti-müllerian hormone (AMH) production, follicular stimulating hormone (FSH) levels, decreased plasma levels of estradiol, or any combination thereof. Therefore, in some embodiments, selection of women about to undergo, currently undergoing, or who have previously undergone a chemotherapy treatment with these characteristics or risks for developing these characteristics can be considered in determination of use of a treatment comprising administration of a melatonin receptor agonist.

### Methods for Use of Melatonin Receptor Agonist Pharmaceutical Compositions

Described herein are methods of use of pharmaceutical compositions formulated to deliver a melatonin receptor agonist to tissues in the endocrine, reproductive, metabolic, or immune systems, or in any combination thereof. In some embodiments, the methods comprise local delivery of pharmaceutical compositions described herein to the cells and tissues where the pharmaceutical compositions are administered. In some embodiments, the methods comprise systemic delivery of pharmaceutical compositions described herein to a subject with reduced CNS accumulation of melatonin receptor agonist compared to CNS accumulation of an equivalent amount of melatonin delivered systemically. In some embodiments, the reduced CNS accumulation of melatonin receptor agonist is due to reduce passive diffusion, increased active transport, increased CSF expulsion via BBB egress transporters, increased CNS metabolism, increase CNS clearance, or modulated protein binding of melatonin receptor agonist compared to an equivalent amount of melatonin delivered systemically. In some embodiments, reduced CNS accumulation of melatonin receptor agonist from the pharmaceutical compositions described herein results in effective delivery of melatonin receptor agonist to target tissues without substantially inducing unwanted function, metabolism, or signal transduction in CNS tissues.

Further examples of the use can include a coating containing a melatonin receptor agonist described herein. In some embodiments, the coating can include coating an article such as a medical device. In some cases, the medical device can be an implantable medical device.

### Methods of Administration

Described herein are methods of administration of pharmaceutical compositions formulated to deliver a melatonin receptor agonist to tissues in the endocrine, reproductive, metabolic, vascular, skeletal, integumentary or immune systems, or in any combination thereof. In some aspects, the method of administration delivers a melatonin receptor agonist to peripheral tissues. In some embodiments, the method comprises local delivery near a target tissue. In some embodiments, the target tissue comprises cervical tissue, myometrial tissue, or oviductal tissue, or any combination thereof. In some embodiments, the local delivery comprises local injection. In some embodiments, the local delivery comprises transdermal or subcutaneous delivery. In some embodiments, the local delivery comprises delivery through a drug delivery device. In some embodiments, the local delivery comprises delivery through a vaginal ring, a vaginal gel, a vaginal film, a vaginal tablet, a pessary, a suppository, or a patch. In some embodiments, the local delivery comprises intrauterine or intravaginal delivery. In some embodiments, the local delivery results in a higher concentration of melatonin receptor agonist near the site of delivery than at a peripheral location farther away from the site of local delivery. In some embodiments, local delivery comprises delivery by a device. In some embodiments, the device comprises an intrauterine device. In some embodiments, delivery by a device that allows or enables time-controlled release of the pharmaceutical composition. In some embodiments, time-controlled release comprises remote-controlled release. In some embodiments, time-controlled release permits release from an inserted chronic device that delivers the pharmaceutical composition at a desired frequency or at a desired duration of administration. In some embodiments, the desired frequency of administration is a diurnal frequency. In some embodiments, the desired frequency of administration avoids somnolence effects of the melatonin receptor agonist. In some embodiments, the pharmaceutical composition is delivered to result in continuous exposure of the melatonin receptor agonists to a plurality of target cells. In some embodiments, a desired pattern of continuous or diurnal daily exposure to melatonin receptor agonist to address multiple phenotypes of PCOS may differ among indications. In some embodiments, methods of administration comprising delivery of pharmaceutical compositions described herein by vaginal gels or drug delivery device (e.g., vaginal rings or intrauterine devices) can achieve superior control of the symptoms of PCOS that derive from the endocrine, inflammatory and oxidative stress pathways and avoid the sleep-inducing effects that accompany orally administered melatonin agonists that achieve high concentrations in the CNS.

Described herein are methods of administration of pharmaceutical compositions formulated to deliver a melatonin receptor agonist to peripheral target tissues that have reduced capacity to accumulate in the CNS. In some embodiments, the methods comprise oral administration of the pharmaceutical composition formulation. In some embodiments, the methods comprise a diminished permeability of the pharmaceutical composition formulation across blood-brain barrier. In some embodiments, the methods comprise a diminished active transport inside the brain through the BBB. In some embodiments, the methods comprise an increased egress outside the brain through the BBB. In some embodiments, the methods comprise an increased metabolism and/or clearance of melatonin receptor agonist in the CNS. In some embodiments, the methods comprise an increased binding to proteins in the CNS that decrease the free concentration of melatonin receptor agonist. In some embodiments, less than about 50%, 45%, 40%, 35%, 30%, 25%, 22%, 20%, 18%, 15%, 13%, 10%, 9%, 8%, 7%, 6%, 5%, 4.5%, 4%, 3.5%, 3%, 2.5%, 2%, 1.5%, 1%, 0.75%, 0.5%, 0.3%, 0.2%, 0.1%, 0.05%, 0.02%, 0.01%, 0.005%, or 0.001% of the melatonin receptor agonist contained within the pharmaceutical composition accumulates in the CNS of the subject. In some embodiments, the method of administration reduces the capacity of the melatonin receptor agonist to accumulate in the CNS and thereby allows for a broader therapeutic dosage range for target cells in peripheral tissues. In some embodiments, the broader therapeutic dosage range for target cells in peripheral tissues may not impact or impairs one or more CNS function. In some embodiments, the broader therapeutic dosage range for target cells in peripheral tissues may mitigate potential side effects in the CNS such as somnolence, defects in sleep patterns, circadian rhythms, motor control, or memory extinction. In some embodiments, the reduced capacity of the melatonin receptor agonist to cross the blood-brain barrier may allow for a higher systemic concentration of melatonin receptor agonist while maintaining a favorable safety profile. In some embodiments, the method of administration comprises delivery by intravenous injection. In some embodiments, the reduced capacity of the melatonin receptor agonist to accumulate in the CNS may allow for delivery by intravenous injection. In some embodiments, the reduced capacity of the melatonin receptor agonist to accumulate in the CNS may allow for delivery by oral administration. In some embodiments, the melatonin receptor agonist is delivered to a target tissue in an amount that is therapeutically effective. In some embodiments, the melatonin receptor agonist is delivered to a target cell in an amount that is therapeutically effective.

### Treatment Regimens

Described herein are methods of administration of pharmaceutical compositions described herein that comprise a treatment regimen. In some embodiments, the method comprises a treatment regimen delivering a pharmaceutical composition described herein according to the needs of a subject. In some embodiments, the method comprises a treatment regimen delivering a therapeutically effective amount of a pharmaceutical composition described herein according to the needs of a subject. In some embodiments, the treatment regimen comprises a single administration. In some embodiments, the treatment regimen comprises at least one administration. In some embodiments, the treatment regimen comprises 1, 2, 3, 4, 5, 6, 7, 8, 9, 10, 11, 12, 13, 14, 15, 16, 17, 18, 19, 20 or more administrations. In some embodiments, an administration comprises delivery of an effective amount of melatonin receptor agonist to target cells. In some embodiments, the effective amount contained within one administration is an effective amount for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 35, 42, 49, 56, 60, 61, 62, or 90 days. In some embodiments, one administration comprises an effective amount for at least 1 week, at least 2 weeks, at least 4 week, at least 2 months, or at least 6 months. In some embodiments, the method further comprises a treatment cycle. In some embodiments, the treatment cycle comprises 1, 2, 3, 4, 5, 6, 7, 8, 9, 10, 11, 12, 13, 14, 15, 16, 17, 18, 19, 20 or more administrations of the pharmaceutical composition. In some embodiments, the treatment regimen comprises a plurality of treatment cycles. In some embodiments, the plurality of treatment cycles is 1, 2, 3, 4, 5, 6, 7, 8, 9, or 10 more treatment cycles. In some embodiments, a treatment cycle comprises a length of time. In some embodiments, the length of time comprises 1, 2, 3, 4, 5, 6, 7, 8, 9, 10, 11, 12, 13, 14, 15, 16, 17, 18, 19, 20, 21, 22, 23, 24, 30, or 36 months. In some embodiments, the melatonin receptor agonist is delivered to a target tissue over a period of time that is therapeutically effective. In some embodiments, the melatonin receptor agonist is delivered to a target cell over a period of time that is therapeutically effective.

In some aspects, the methods of administration of pharmaceutical compositions described herein comprise initiating administering at various points of time according to a treatment regimen. In some embodiments, the subject is pretreated by initiating administering of the pharmaceutical composition prior to undergoing a chemotherapy treatment. In some embodiments, the pretreatment is initiated at least about 15 days, 14 days, 13 days, 12 days, 11 days, 10 days, 9 days, 8 days, 7 days, 6 days, 5 days, 4 days, 3 days, 2 days, 1 day, 18 hours, 12 hours, 8 hours, 6 hours, 4 hours, 2 hours, or 1 hour prior to initiation of a chemotherapy treatment in the subject. In some embodiments, the subject is concurrently treated by initiating administering of the pharmaceutical composition while undergoing a chemotherapy treatment. In some embodiments, the subject is treated by maintaining administering of the pharmaceutical composition following the completion of a chemotherapy treatment.

In some aspects, the methods of administration of pharmaceutical compositions described herein comprise administrating at various points of time according to a treatment regimen. In some embodiments, pharmaceutical compositions are administered at certain times of day or night. In some embodiments, pharmaceutical compositions are administered according to a circadian rhythm of the subject. In some embodiments, pharmaceutical compositions are administered at times in a circadian rhythm of the subject in which plasma melatonin levels are low. In some embodiments, pharmaceutical compositions are administered at a time in a circadian rhythm of the subject in which a plasma melatonin level of the subject is below about 50 pg/mL, 45 pg/mL, 40 pg/mL, 35 pg/mL, 30 pg/mL, 25 pg/mL, 20 pg/mL, 15 pg/mL, 14 pg/mL, 13 pg/mL, 12 pg/mL, 11 pg/mL, 10 pg/mL, 9 pg/mL, 8 pg/mL, 7 pg/mL, 6 pg/mL, or 5 pg/mL. In some embodiments, pharmaceutical compositions are administered at a time throughout a day according to a presumed circadian rhythm of the subject. In some embodiments, pharmaceutical compositions are administered at about 12:00 AM, 1:00 AM, 2:00 AM, 3:00 AM, 4:00 AM, 5:00 AM, 6:00 AM, 7:00 AM, 8:00 AM, 9:00 AM, 10:00 AM, 11:00 AM, 12:00 PM, 1:00 PM, 2:00 PM, 3:00 PM, 4:00 PM, 5:00 PM, 6:00 PM, 7:00 PM, 8:00 PM, 9:00 PM, 10:00 PM, or 11:00 PM according to the current time zone of the subject. In some embodiments, pharmaceutical compositions are administered every day, every 2 days, every 3 days, every 7 days, every 28 days, or every 2 months. In some embodiments, the method administration of pharmaceutical compositions comprises continuous administration of melatonin receptor agonist to the subject. In some embodiments, continuous administration of a pharmaceutical composition described herein to the subject comprises a delayed release, a sustained release, an extended release, a prolonged release, or a slow release of melatonin receptor agonist. In some embodiments, local administration of a pharmaceutical composition described herein has a reduced effect on a melatonin receptor mediated CNS behavior compared to an administration of an equivalent amount of melatonin. In some embodiments, the melatonin receptor mediated CNS behavior comprises circadian rhythm, addiction, sleep behavior, motor control, or memory extinction. In some embodiments, local delivery of a pharmaceutical composition described herein may require delivery according to a peripheral circadian rhythm of the subject. In some embodiments, the peripheral circadian rhythm may operate independently of pineal gland-mediated circadian regulation of plasma melatonin levels.

In some aspects, pharmaceutical compositions are administered irrespective of a circadian rhythm of the subject. In some embodiments, the methods of administration of pharmaceutical compositions described herein comprise greater accumulation in peripheral tissues than in CNS tissues of melatonin receptor agonist. In some embodiments, the greater accumulation in peripheral tissues than in CNS tissues of melatonin receptor agonist decouples melatonin receptor mediated CNS behavior relating to plasma melatonin concentration from effects from administration of melatonin receptor agonist. In some embodiments, the methods described herein comprise peripheral tissue restriction of melatonin receptor agonist. In some embodiments, peripheral tissue restriction of melatonin receptor agonist decouples melatonin receptor mediated CNS behavior relating to plasma melatonin concentration from effects from administration of melatonin receptor agonist. In some embodiments, pharmaceutical compositions described herein may be administered according to a time schedule irrespective of circadian-regulated plasma melatonin levels in the subject. In some embodiments, methods of administering pharmaceutical compositions described herein which result in reduced CNS accumulation of melatonin receptor agonist allow for administration at any time of day. In some embodiments, methods of administering pharmaceutical compositions described herein which result in reduced CNS accumulation of melatonin receptor agonist allow for administration at any time of day without significant unwanted drug response in the CNS. In some embodiments, methods of administering pharmaceutical compositions described herein which result in reduced CNS accumulation of melatonin receptor agonist allow for administration irrespective of circadian-regulated plasma melatonin levels in the subject.

### Methods Producing a Desirable Pharmacodynamic Response

In some aspects, the methods described herein comprise administration of a pharmaceutical composition described herein that produces a desirable PD profile within the subject. In some embodiments, the method yields a PD profile within a subject that delivers melatonin receptor agonist to target cells outside of the CNS. In some embodiments, the method yields a PD profile within a subject that delivers an effective amount of melatonin receptor agonist to target cells outside of the CNS. In some embodiments, the target cells outside of the CNS comprise cells of the endocrine, reproductive, metabolic, vascular, skeletal, integumentary or immune systems, or any combination thereof. In some embodiments, the target cells comprise ovarian cells. In some embodiments, the melatonin receptor agonist functions as a direct antioxidant. In some embodiments, the melatonin receptor agonist directly oxidizes ROS within target cells, RNS within target cells, or a combination thereof. In some embodiments, the melatonin receptor agonist directly oxidizes ROS within mitochondria of target cells, RNS within mitochondria of target cells, or a combination thereof. In some embodiments, the melatonin receptor agonist directly oxidizes ROS extracellular to target cells, RNS extracellular to target cells, or a combination thereof. In some embodiments, the melatonin receptor agonist functions to locally upregulate melatonin receptor signal transduction. In some embodiments, the local upregulation of melatonin receptor signal transduction occurs in target cells comprising ovarian cells, oocytes, granulosa cells, theca cells, ovarian stromal cells, ovarian epithelial cells, Fallopian tube cells, uterine cells, endometrial cells, myometrial cells, cervical cells, adrenal gland cells, pancreatic cells, thyroid gland cells, parathyroid gland cells, skeletal muscle cells, liver cells, white adipose tissue cells, brown adipose tissue cells, osteoblasts, osteoclasts, endothelial cells, hair follicle cells, lymphocytes, neutrophils, monocytes, or macrophages, or progenitors of any of the above cells types. In some embodiments, the local upregulation of melatonin receptor signal transduction increases expression of a plurality of cellular antioxidants. In some embodiments, the plurality of cellular antioxidants comprises superoxide dismutase, glutathione reductase, glutathione peroxidase, or catalase, or any combination thereof. In some embodiments, the plurality of cellular antioxidants comprise the genes SOD1, SOD2, GSR, GPX1, GPX2, GPX3, GPX4, GPX5, GPX6, GPX7, GPX8, or CAT. In some embodiments, the local upregulation of melatonin receptor signal transduction increases activity of a plurality of cellular antioxidants. In some embodiments, the plurality of cellular antioxidants comprises superoxide dismutase, glutathione reductase, glutathione peroxidase, or catalase, or any combination thereof. In some embodiments, the local upregulation of melatonin receptor signal transduction results in a change in intracellular cyclic nucleotides in a plurality of target cells. In some embodiments, the intracellular cyclic nucleotides comprise cAMP, cGMP, IP₃, or DAG, or any combination thereof. In some embodiments, IP₃ and DAG serve as messengers for activation of the PI3K/AKT pathway. In some embodiments, the local upregulation of melatonin receptor signal transduction results in a change in cytosolic calcium levels in a plurality of target cells. In some embodiments, the local upregulation of melatonin receptor signal transduction results in a change in mitochondrial calcium levels in a plurality of target cells. In some embodiments, the local upregulation of melatonin receptor signal transduction results in activation of PKC subtypes in a plurality of target cells. In some embodiments, the PKC subtypes comprise conventional, novel, or atypical PKC subtypes. In some embodiments, the local upregulation of melatonin receptor signal transduction results in changes in intracellular localization of steroid hormone receptors. In some embodiments, the local upregulation of melatonin receptor signal transduction results in changes in expression of gonadotrophin-releasing hormone receptors, luteinizing hormone receptors, or follicle-stimulating hormone receptors, or any combination thereof. In some embodiments, the changes in expression of hormone receptors comprises increases in expression level of mRNAs coding for hormone receptors. In some embodiments, the local upregulation of melatonin receptor signal transduction results in a change in secretion of progesterone from granulosa cells. In some embodiments, the change in secretion of progesterone comprises elevated secretion of progesterone. In some embodiments, the local upregulation of melatonin receptor signal transduction results in alterations in secretion of a plurality of endocrine hormones comprising insulin, glucagon, somatostatin, pancreatic polypeptide, cholecystokinin, secretin, amylin, gastrin, or thyroxine, or any combination thereof. In some embodiments, the local upregulation of melatonin receptor signal transduction results in activation of MAPK pathway signal transduction. In some embodiments, the MAPK pathway signal transduction pathway activated is the MAPK-JNK/P38 signaling pathway. In some embodiments, the local upregulation of melatonin receptor signal transduction results in activation of a G-protein signaling pathway in a plurality of target cells. In some embodiments, the local upregulation of melatonin receptor signal transduction results in inhibition of adenyl cyclase in a plurality of target cells. In some embodiments, the local upregulation of melatonin receptor signal transduction results in activation of phospholipase C in a plurality of target cells. In some embodiments, the local upregulation of melatonin receptor signal transduction results in a change in lipid metabolism in a plurality of target cells. In some embodiments, the local upregulation of melatonin receptor signal transduction results in a change in follicular development. In some embodiments, the change in follicular development comprises prevention of granulosa cell apoptosis in a plurality of granulosa cells. In some embodiments, the prevention of granulosa cell apoptosis improves follicular development. In some embodiments, the local upregulation of melatonin receptor signal transduction results in improved regulation of blood pressure. In some embodiments, the local upregulation of melatonin receptor signal transduction results in decreased bone density loss. In some embodiments, the local upregulation of melatonin receptor signal transduction results in decreased loss of hair.

### Methods to Modulate a Phenotype

Described herein are methods of administration of pharmaceutical compositions described herein that modulate a phenotype in a subject. In some aspects, the phenotype in the subject is affected by melatonin. In some embodiments, the phenotype affected by melatonin is caused by melatonin system dysfunction. In some embodiments, the phenotype affected by melatonin is susceptible to age-related changes in melatonin distribution. In some embodiments, age-related changes in melatonin distribution comprise decreased melatonin production. In some embodiments, age-related changes in melatonin distribution comprise low melatonin levels in some peripheral tissues and normal or high plasma concentrations of melatonin. In some embodiments, age-related changes in melatonin distribution comprise decreased extrapineal melatonin production. In some embodiments, age-related changes in melatonin distribution comprise decreased ovarian melatonin production. In some embodiments, tissue damage as a result of disease or other insult results in a phenotype affected by melatonin in the subject. In some embodiments, administration of a pharmaceutical composition comprising a melatonin receptor agonist in a subject with a phenotype affected by melatonin results in a slowdown of disease progression. In some embodiments, administration of a pharmaceutical composition comprising a melatonin receptor agonist in a subject with a phenotype affected by melatonin results in a halting of disease progression. In some embodiments, administration of a pharmaceutical composition comprising a melatonin receptor agonist in a subject with a phenotype affected by melatonin results in an improvement in an aspect of a disease.

In some aspects, the phenotype affected by melatonin in the subject comprises impairment in endocrine cells, in reproductive cells, in metabolic cells, in vascular cells, in skeletal cells, in integumentary cells, or in immune cells, or any combination thereof. In some embodiments, the phenotype affected by melatonin in the subject responds to therapy comprising administration of melatonin receptor agonists. In some embodiments, the phenotype in the subject that responds to melatonin receptor agonist therapy comprises an ovarian phenotype, an endometrial phenotype, a vascular phenotype, a skeletal phenotype, an integumentary phenotype, an inflammatory phenotype, a metabolic phenotype, or a liver phenotype. In some embodiments, the ovarian phenotype comprises abnormal or reduced folliculogenesis, oligoovulation, anovulation, ovulatory dysfunction, altered ovarian progesterone production, altered luteal phase production of progesterone, or poor oocyte maturation. In some embodiments, the endometrial phenotype comprises endometriosis, endometriosis-associated ovarian oxidative stress, pelvic pain, abdominal pain, back pain, dysmenorrhea, or adenomyosis. In some embodiments, the inflammatory phenotype comprises low grade systemic inflammatory, local idiopathic inflammation, salpingitis, abnormal immune response, autoimmune damage, or poor embryo development. In some embodiments, the metabolic phenotype comprises elevated insulin resistance, dysregulated glucose homeostasis, excess weight or excess BMI, or increased oxidative stress. In some embodiments, the vascular phenotype comprises abnormal blood pressure, abnormal blood pressure regulation, increased blood pressure, or hypertension. In some embodiments, the skeletal phenotype comprises increased bone density loss, decreased bone density, accelerated bone density loss, or osteoporosis. In some embodiments, the integumentary phenotype comprises increased hair loss, androgenetic alopecia, female pattern hair loss (FPHL), or hair thinning.

In some embodiments, the phenotype in the subject that responds to melatonin receptor agonist therapy comprises an aspect of a particular disease indication or condition. In some embodiments, the aspect of a particular disease indication or condition that responds to melatonin receptor agonist therapy comprises ovulatory dysfunction, luteal insufficiency, PCOS, anovulatory PCOS, insulin resistance in PCOS, glucose tolerance in PCOS, risk of NAFLD in PCOS patients, weight control in PCOS, uterine inflammatory diseases, inflammation in PCOS, hyperandrogenism, hirsutism, diminished uterine receptivity, an ART outcome, abnormal blood pressure, abnormal blood pressure regulation, increased blood pressure, hypertension, bone density loss, decreased bone density, accelerated bone density loss, osteoporosis, increased hair loss, androgenetic alopecia, female pattern hair loss (FPHL), hair thinning, pre-eclampsia, endometriosis, endometriosis-associated infertility, pelvic pain, abdominal pain, back pain, dysmenorrhea, or EACPP.

Described herein are methods of administration of pharmaceutical compositions described herein that may yield an improved ART outcome. In some the methods, the improved ART outcome comprises an improvement in oocyte and embryo maturation and quality. In some embodiments, the improvement in oocyte and embryo maturation and quality is due to increased ROS-scavenging action of melatonin receptor agonist and/or increased melatonin receptor signaling in target tissue. In some embodiments, increased melatonin receptor signaling in target tissue produces an anti-inflammatory effect, an antioxidant effect, or a combination thereof. In some embodiments, the improved ART outcome comprises a modulation of steroidogenesis by the melatonin receptor agonist. In some embodiments, the modulation of steroidogenesis by the melatonin receptor agonist comprises regulation of progesterone production or a decrease in androgen levels.

Described herein are methods of administration of pharmaceutical compositions described herein to a subject in need thereof. In some embodiments, the method comprises delivery of a pharmaceutical composition by oral, transdermal, subcutaneous, intravenous or local administration. In some embodiments, the method comprises delivery of a pharmaceutical composition to the upper reproductive tract. In some embodiments, delivery of a pharmaceutical composition to the upper reproductive tract allows for elevated local tissue concentration of melatonin receptor agonist without raising plasma concentration of melatonin receptor agonist to about the same extent. In some embodiments, delivery of a pharmaceutical composition to the upper reproductive tract comprises an local tissue concentration of melatonin receptor agonist that is at least 1%, 2%, 3%, 4%, 5%, 6%, 7%, 8%, 9%, 10%, 12%, 14%, 15%, 16%, 18%, 20%, 22%, 25%, 27%, 30%, 32%, 35%, 37%, 40%, 45%, 50%, 55%, 60%, 65%, 70%, 75%, 80%, 85%, 90%, 95%, 100%, 110%, 120%, 130%, 140%, 150%, 175%, 200%, 250%, 300%, 350%, 400%, 450%, 500%, 600%, 700%, 800%, 900%, 1000%, 2000%, 3000%, 4000%, 5000%, or 10000% greater than that of plasma concentration of the melatonin receptor agonist. In some embodiments, delivery of a pharmaceutical composition to the upper reproductive tract comprises an local tissue concentration of melatonin receptor agonist that is at least 1%, 2%, 3%, 4%, 5%, 6%, 7%, 8%, 9%, 10%, 12%, 14%, 15%, 16%, 18%, 20%, 22%, 25%, 27%, 30%, 32%, 35%, 37%, 40%, 45%, 50%, 55%, 60%, 65%, 70%, 75%, 80%, 85%, 90%, 95%, 100%, 110%, 120%, 130%, 140%, 150%, 175%, 200%, 250%, 300%, 350%, 400%, 450%, 500%, 600%, 700%, 800%, 900%, 1000%, 2000%, 3000%, 4000%, 5000%, or 10000% greater than the concentration of the melatonin receptor agonist accumulated in the CNS. In some embodiments, the greater local tissue concentration of melatonin receptor agonist avoids the occurrence or extent of an unwanted drug response in the CNS of a subject. In some embodiments, the greater local tissue concentration of melatonin receptor agonist avoids the occurrence or extent of an unwanted drug response in the CNS of a subject and delivers a therapeutical effective amount of the melatonin receptor agonist to a target tissue. In some embodiments, the method comprises local delivery of the pharmaceutical composition by a drug delivery device. In some embodiments, the method comprises local delivery of the pharmaceutical composition by a vaginal gel, a vaginal ring, a vaginal tablet, a pessary, a suppository, a patch, or an intrauterine device. In some embodiments, local delivery comprises local injection into a region nearby a target tissue.

Described herein are methods of administration of pharmaceutical compositions described herein to a subject in need thereof. In some aspects, the method comprises delivery of a pharmaceutical composition by systemic delivery. In some embodiments, the systemic delivery comprises oral, transdermal, subcutaneous, intravenous or local administration. In some embodiments, the pharmaceutical composition has a diminished ability to accumulate in the CNS. In some embodiments, the diminished ability to accumulate in the CNS comprises at least 1%, 2%, 3%, 4%, 5%, 6%, 7%, 8%, 9%, 10%, 12%, 14%, 15%, 16%, 18%, 20%, 22%, 25%, 27%, 30%, 32%, 35%, 37%, 40%, 45%, 50%, 55%, 60%, 65%, 70%, 75%, 80%, 85%, 90%, 95%, 100%, 110%, 120%, 130%, 140%, 150%, 175%, 200%, 250%, 300%, 350%, 400%, 450%, 500%, 600%, 700%, 800%, 900%, 1000%, 2000%, 3000%, 4000%, 5000%, or 10000% reduction in the melatonin receptor agonist ability to accumulate in the CNS as compared to CNS accumulation of an equivalent dose of melatonin. In some embodiments, the reduction in the melatonin receptor agonist ability to accumulate in the CNS avoids the occurrence or extent of an unwanted drug response in the CNS of a subject. In some embodiments, the reduction in the melatonin receptor agonist ability to accumulate in the CNS avoids the occurrence or extent of an unwanted drug response in the CNS of a subject and delivers a therapeutical effective amount of the melatonin receptor agonist to a target tissue. In some embodiments, an aspect of the formulation of the pharmaceutical composition results in the reduction in the melatonin receptor agonist ability to accumulate in the CNS. In some embodiments, a modification to the melatonin receptor agonist results in the reduction in the melatonin receptor agonist ability to accumulate in the CNS. In some embodiments, the pharmaceutical composition comprises a melatonin receptor agonist-conjugate. In some embodiments, the conjugate comprises a hydrophilic molecule, a carbohydrate molecule, a peptide, or a synthetic molecule.

In some aspects, the method described herein yields a desirable PK profile of the pharmaceutical composition within the subject. In some embodiments, the PK profile will comprise a higher level of melatonin receptor agonist bioavailability near target cells outside of the CNS. In some embodiments, the PK profile will comprise a lower level of melatonin receptor agonist bioavailability in the CNS than near target cells outside of the CNS. In some embodiments, the target cells outside of the CNS comprise cells involved in endocrine function, cells involved in reproductive function, cells involved in metabolic function, cells involved in vascular function, cells involved in skeletal function, cells involved in integumentary function, or cells involved in immune function. In some embodiments, the target cells outside of the CNS comprise cells of the upper reproductive tract. In some embodiments, the cells of the upper reproductive tract comprise ovarian cells, egg cells, Fallopian tube cells, uterine cells, endometrial cells, myometrial cells, or cervical cells. In some embodiments, the cells involved in reproductive function comprise ovarian cells, oocytes, granulosa cells, theca cells, ovarian stromal cells, ovarian epithelial cells, Fallopian tube cells, uterine cells, endometrial cells, myometrial cells, or cervical cells. In some embodiments, the target cells outside of the CNS involved in endocrine function comprise cells of the adrenal glands, pancreatic cells, thyroid gland cells, parathyroid gland cells, or ovarian cells. In some embodiments, the target cells outside of the CNS involved in metabolic function comprise skeletal muscle cells, liver cells, pancreatic cells, or adipose tissue cells. In some embodiments, the target cells outside of the CNS involved in immune function comprise lymphocytes, neutrophils, monocytes, or macrophages. In some embodiments, the target cells outside of the CNS involved in immune function comprise the progenitors of lymphocytes, neutrophils, monocytes, or macrophages. In some embodiments, the lymphocytes comprise T-cells, B-cells, or NK cells. In some embodiments, the target cells involved in vascular function comprise endothelial cells. In some embodiments, the target cells involved in skeletal function comprise osteoblasts and osteoclasts. In some embodiments, the target cells involved in integumentary function comprise hair follicle cells.

### Kits

Disclosed herein are kits. A kit can comprise a melatonin receptor agonist, salt thereof, formulation, or composition described herein. In some aspects, melatonin receptor agonist, formulation, or composition can be packaged in a container. In some embodiments, a kit can further comprise instructions that direct administration of a melatonin receptor agonist, or of a unit dose of melatonin receptor agonist or formulation to a subject. In some embodiments, a kit can comprise melatonin receptor agonist disclosed herein and instructions for the use thereof. In some embodiments, the instructions for use designate one or more indications in a subject in need of treatment, wherein the one or more indications comprise polycystic ovary syndrome (PCOS), endometriosis, menopause, chemotherapy-induced ovarian dysfunction (CIOD), chemotherapy-induced ovarian failure (CIOF), or inflammation of a reproductive tract, or a combination thereof. In some embodiments, the kit further comprises a drug delivery device for administering the pharmaceutical composition to a subject. In some embodiments, the drug delivery device is a vaginal ring, a vaginal tablet, a pessary, a suppository, a patch, or an intrauterine device. In some embodiments, the drug delivery device is configured to be placed in proximity to a reproductive organ of the subject. In some embodiments, the drug delivery device is the intrauterine device and configured to be placed in proximity to a reproductive organ of the subject. In some embodiments, the intrauterine device is configured for time-controlled release or remote-controlled release of one or a plurality of doses of the pharmaceutical composition formulated for delayed release, sustained release, extended release, prolonged release, or slow release.

Methods of making a kit can include placing a melatonin receptor agonist, salt thereof, formulation, or composition described herein in a container for packaging. A method can further comprise an inclusion of instructions for use. In some cases, instructions for use can direct administration of a melatonin receptor agonist, or of a unit dose of a melatonin receptor agonist or formulation to a subject.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular. Accordingly, terms such as “first” and “second” do not necessarily imply priority, order of importance, or order of acts.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease. Designation as a “subject,” “individual” or “patient” does not necessarily entail supervision of a medical professional.

The term “in vivo” is used to describe an event that takes place in a subject's body.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to ±10% of a stated number or value.

Throughout this application, various aspects may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The terms “increased”, “increasing”, or “increase” are used herein to generally mean an increase by a statically significant amount. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease by a statistically significant amount. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

The terms “follicle” and “ovarian follicle” refer to aggregations of cells found in the ovary containing an oocyte or immature oocyte that develop inside the follicle (e.g., comprising a densely packed shell of somatic cells that contains an immature oocyte).

The term “folliculogenesis” refers to a process, or any stage thereof, in which ovarian follicles containing (immature) oocytes mature, including any stage of the progression from primordial follicle to preovulatory follicle and/or from immature oocyte to ovum.

The term “oocyte” refers to a cell capable of maturing to a female haploid egg cell (ovum) by meiosis.

The terms “menopause symptoms” and “menopausal symptoms” refer to symptoms and diseases that occur in women before, during and after menopause caused at least in part by ovarian aging, hormonal changes, and/or other biological processes related to menopause.

The term “ovarian reserve” refers to the capacity of the ovary to provide egg cells that are capable of fertilization resulting in a healthy and successful pregnancy and ovarian follicle cells that are capable of generating ovarian hormones and signaling molecules that underly the endocrine function of the ovary.

The section headings used herein are for organizational purposes only and are not to be construed as limiting the subject matter described.

While preferred aspects of the present invention have been shown and described herein, it will be obvious to those skilled in the art that such aspects are provided by way of example only. It is not intended that the invention be limited by the specific examples provided within the specification. While the invention has been described with reference to the aforementioned specification, the descriptions and illustrations of the aspects herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the aspects of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### EXAMPLES

The following illustrative examples are representative of embodiments of the stimulation, systems, and methods described herein and are not meant to be limiting in any way.

### Example 1: In Vitro Pharmacology: Receptor Binding Assay (IC50) and In Vitro Potency Assay (EC50)

Ligand binding assays are performed to determine the concentrations producing half-maximal inhibition of control specific binding (IC50) at MT₁ and MT₂. Compounds are incubated in a serial dilution with the cells and the ligand. Samples are incubated for a predetermined time at a predetermined temperature. Nonspecific binding is evaluated through the incubation with melatonin or known analog at 1 μM for MT₁ and MT₂. After incubation, the % of ligand binding inhibition is quantified. A dose-response to the compound is measured in a cell functional assay measuring CAMP levels. EC50 is the half-maximal agonism response at MT₁ and MT₂.

### Example 2: In Vitro Selectivity Screening

Selectivity of the compounds for melatonin receptors against a GPCR panel is tested using SelectScreen Cell-based GPCR Profiling by ThermoFisher and/or Eurofins DiscoveRx platform. Of particular interest in the selectivity determinations are those G-protein coupled receptors with known similarity to MT₁ and MT₂ and known ligands to use in assessing selectivity. For comparison, the similarity of other species melatonin receptors are included in Table 3 below.

TABLE 3
Percent similarity of G-protein coupled receptors to MT₁ and MT₂

| MT₁ Receptor | % similarity | MT₂ Receptor | % similarity |
|---|---|---|---|
| NPY5R | 20 | | |
| MCHR2 | 18.5714 | GPR50 | 47.4286 |
| MCHR1 | 18.8571 | PRLHR | 22.2857 |
| GPR75 | 18.8571 | NPY4R2 | 21.7143 |
| NPY1R | 22 | NPY4R | 21.7143 |
| TACR1 | 21.1429 | NPYR2 | 23.7143 |
| TACR3 | 22.2857 | TACR2 | 21.1429 |
| PROKR2 | 20.8571 | PROKR1 | 20.2857 |

### Example 3: Safety Screening

Safety evaluation compatible with ICH S2A/B and ICHS7 guidelines include functional observable behaviors (FOB) (e.g. modified Irwin test) and are used to test the impact of the compound on the CNS, evaluation of risks to respiratory function, and cardiovascular assessment including the in vitro hERG assay (to identify potential risk of QT interval prolongation) and in vivo evaluation as commonly implemented for ICH safety evaluation.

### Example 4: MDR1 Permeability Cell-Based Assay

MDR1-expressing cells (e.g., MDR1-MDCK cells) are seeded onto a trans well filter insert and incubated with a solution containing the melatonin receptor agonist with or without MDR1 inhibitor. After incubation for a predetermined time, samples from the apical and basolateral compartments are tested for the concentration of the compound.

### Example 5: Synthetic Gastric Fluid Fed Vs Fasted

Drug dissolution and absorption in the physiological environment of the gastrointestinal (GI) tract is evaluated in biorelevant dissolution media. In this instance, drug refers to a selected pharmaceutical composition comprising a melatonin receptor agonist.

### Example 6: In Vitro MTNR1A/B Activation Assays

In vitro assays can determine effectiveness of a compound (e.g., a melatonin receptor agonist) in MTNR1A/B activation. The effects of the compound on melatonin signaling can be assessed by testing the ability of the compound to increase the following readouts of melatonin signaling. Target validation can be investigated on melatonin-mediated metabolic processes that have been characterized in cell lines. These include:
Treatment of C2C12 cell line with receptor agonists will stimulate glucose uptake via activation of the PI3K pathway. This process will be inhibited by co-treating cells with luzindole.
Treatment of insulin resistant 3T3-Lladipocytes (induced by exposure to palmitic acids) with receptor agonists will stimulate an increase in glucose uptake and GLUT4 levels and a decrease in phosphorylation of IRS1 at Ser307 (inhibitory).
Treatment of rat insulin-secreting cells (INS1) with receptor agonists will inhibit insulin secretion. This process will be diminished by the use of luzindole.
Treatment of granulosa cells (KGN or primary cells) with melatonin will increase expression of several cyclic nucleotide phosphodiesterase isoforms, phosphorylation of protein kinase B, expression of SIRT1, and to inhibit measures of oxidative phosphorylation and mitochondrial dysfunction [source: PMCID: PMC8950389; PMCID: PMC8582167].
Treatment of macrophages (RAW264.7 or primary cells) with LPS induces the expression of pro-inflammatory molecules (e.g. TNFα, IL1B, IL6, IL8) and melatonin reduces their LPS-induced expression.

### Example 7: In Vivo Pharmacology

Concentration of the compounds in the brain, cerebrospinal fluid (CNF), plasma and peripheral tissues at serial time points is determined following oral and/or i.v. and/or local administration of the compound to rats and/or mice. Samples are analyzed via LC-MS and/or microdialysis. Bound and free concentration of the compound in different samples at different time points are evaluated.

### Example 8: In Vivo Disease Models

Melatonin has been shown to improve metabolic and reproductive traits in PCOS animal models. A compound functioning as a melatonin receptor agonist will cause one or more of the following phenotypic changes in PCOS rat/mouse models (both DHT-induced or continuous light-stimulated models have been used to test effects of melatonin):
Interrupt prolonged estrus cycle
Increase the number of corpora lutea
Decrease the number of cyst-like follicles
Decrease levels of testosterone and AMH
Improved glucose tolerance and increased glucose uptake
Decreased leptin levels
Relieved hyperinsulinemia
Decreased serum TNFα levels
Increased antioxidant capacity (e.g. superoxide dismutase, catalase and glutathione-reductase activities, reducing power (GSH/GSSG ratio).

The following PCOS animal models are provided for determining the effects of melatonin receptor agonist treatment.

In a PCOS mouse model, mice are treated with a local injection of melatonin receptor agonist into the myometrium (either ultrasound guided, or visually-guided after incision in peritoneum) or saline vehicle at Day 0. Mice are separated into Groups 1-7 according to this example and treatment groups follow the animal model experimental group and treatment dose design listed in Table 4. Mice are assayed for the above features on Day 1, 2, 3, 7, 10, 14, 17, 21, 24, 28, and/or 35.

### PCOS Induction in Androgen-Induced Model:

For PCOS induction in an androgen-induced model using rats, the follow protocol is implemented. PCOS is induced in rats by daily subcutaneous administration of testosterone propionate (TP) (10 mg/kg) for 35 days. Groups 2-7 have disease induction. Group 1 is a vehicle control.

Prophylactic treatment is administered from Day 1 to Day 35 of the study design. Rats are randomized by birth weight (BW) on Day 0 and allocated to Groups 1-7. All treatments (on Day 1 through Day 35) start when the rats are 21 days old and are given once daily by oral gavage for 35 consecutive days. For animals in treatment groups receiving once daily (QD) treatment, melatonin analog is administered via oral gavage 1 hour after onset of darkness. For animals in treatment groups receiving twice daily (BID) treatment, melatonin analog is first administered via oral gavage 1 hour after onset of darkness, and second, administered via oral gavage about 5 hours after the first administration. For the androgen-induced PCOS model, animals are maintained on a 12 hour light/12 hour dark daily light schedule.

### PCOS Induction in Light-Induced (or Darkness-Induced) Model:

PCOS is induced in rats by exposure to constant light (or constant darkness) for 8 weeks. Groups 2-7 have disease induction. Group 1 is a vehicle control.

All treatments start when the rats are 5 weeks old and given once daily for 8 weeks. Prophylactic treatment is administered on Day 1 to Day 56 (randomization by BW on Day 0) of the study design. Melatonin analog is administered via oral gavage at the same time of day on treatment days (Day 1 through Day 56).

For both the PCOS induction in androgen-induced model and the PCOS induction in light-induced (or darkness-induced) model, Groups 1-7 receive the treatments according to Experimental Group listed in Table 4 on study design days of treatment. Group 1 animals receive vehicle control treatment of Sunflower oil according to study design days of treatment. Group 2 androgen-induced PCOS induction model animals receive TP administration for disease induction but no treatment. Group 2 PCOS induction in light-induced (or darkness-induced) model animals are subject to PCOS induction but receive no treatment. Group 3 animals receive daily metformin hydrochloride treatment as a positive control for the treatment of metabolic symptoms according to study design days of treatment. Group 4 animals receive a low dose melatonin analog treatment once daily (QD) according to study design days of treatment. Group 5 animals receive a high dose melatonin analog treatment once daily (QD) according to study design days of treatment. Group 6 animals receive a melatonin analog treatment twice daily (BID) according to study design days of treatment. Group 7 animals receive a daily melatonin treatment according to study design days of treatment.

TABLE 4
PCOS model experimental group and treatment dose

| Experimental Group | Treatment Dose |
|---|---|
| 1—Non disease control | N/A |
| 2—PCOS Disease Control | N/A |
| 3—PCOS + Metformin hydrochloride | 500 mg/kg body weight |
| 4—PCOS + Melatonin analog QD low | 30 mg/kg body weight |
| 5—PCOS + Melatonin analog QD high | 60 mg/kg body weight |
| 6—PCOS + Melatonin analog BID | 30 mg/kg body weight |
| 7—PCOS + Melatonin | 2 mg/kg body weight |

The following routes of administration are used according to the agent administered to the subjects: Oral gavage (Metformin, Melatonin, Melatonin analog), subcutaneous (Sunflower oil, TP). The melatonin analog administered to animals in Groups 4-6 on study design days of treatment is N-[2-(5-Chloro-2,6-dimethoxybenzoimidazol-1-yl)ethyl]acetamide (ACH000-143), (compound 10b). Treatment dose for each treatment administration for Groups 3-7 is listed in Table 4.

Androgen-induced PCOS induction model animals from Groups 1-7 are assayed for the PCOS phenotypic changes listed above in this example on Day 1, 2, 3, 7, 10, 14, 17, 21, 24, 28, and/or 35. At end of study, remaining animals from Groups 1-7 are assayed for extent of PCOS phenotypic changes and symptoms listed above in this example.

PCOS induction in light-induced (or darkness-induced) model animals from Groups 1-7 are assayed for the PCOS phenotypic changes listed above in this example on. Day 1, 2, 3, 7, 10, 14, 17, 21, 24, 28, 35, 42, 49 and/or 56. At end of study, remaining animals from Groups 1-7 are assayed for extent of PCOS phenotypic changes and symptoms listed above in this example. Animals are also assayed according to the parameters listed in Example 7: In vivo pharmacology. Table 5 lists expected results in PCOS Control Animals and Experimental Groups receiving melatonin analog therapy.

TABLE 5
expected results in PCOS induction models at end of study.

| | | PCOS with |
|---|---|---|
| | | melatonin analog |
| Endpoints | PCOS | treatment |
| Body weight (g) | Increased | Decreased |
| BMI | Increased | Decreased |
| AC/TC ratio | Increased | Decreased |
| IAF (g) | Increased | Decreased |
| Ovary weight (g) | Decreased | Increased |
| Ovary volume | Decreased | Increased |
| Uterus weight (g) | Decreased | Increased |
| Corpora lutea (% follicles) | Decreased | Increased |
| Cysts (% follicles) | Increased | Decreased |
| LH/FSH | Increased | Decreased |
| Insulin (uIU/mL) | Increased | Decreased |
| Testosterone (ng/dL) | Increased | Decreased |
| CRP (mg/dL) | Increased | Decreased |
| Glucose (mg/dL) | Increased | Decreased |
| HOMA-IR | Increased | Decreased |

An in vivo pharmacology study is conducted in one or more PCOS induction models. Concentration of compounds assayed in the brain, cerebrospinal fluid (CNF), plasma and peripheral tissues at serial time points is determined following oral and/or i.v. and/or local administration of the melatonin analog treatment to rats and/or mice. Samples are analyzed via LC-MS and/or microdialysis. Bound and free concentration of the melatonin analog in different samples at different time points are evaluated. The in vivo pharmacology study is conducting using i) subjects with no disease induction (e.g., wild-type mice or wild-type rats), and ii) subjects with PCOS induction as indicated in this example. The following data are collected during the pharmacology study:
In life anthropometrical parameters (BMI, abdominal circumference to thoracic circumference (AC/TC) ratio)
In life colpocytological examination (estrus cycle)
PK profiling (T0, 30′, 1 h, 2 h, 4 h, 8 h, 24 h)
Serum biomarkers: FSH, LH, Testosterone, CRP, insulin, glucose
Histopathology: Ovary, Uterus, and IAF (gross morphology+ovarian histology and follicle count) at end of study

Expected results in PCOS induction models at end of study are listed in Table 5.

Conclusions: Prophylactic treatment with the melatonin analog in subjects prevents the induction of an extent of PCOS phenotypic changes and symptoms compared to PCOS disease control subjects. Prophylactic and concurrent treatment with a melatonin analog in subjects improves induced PCOS phenotypic changes and symptoms compared to PCOS disease control subjects. Prophylactic and concurrent treatment with a melatonin analog in subjects improves at least 1, 2, 3, 4, 5, 6, 7, 8, 9, 10, 11, 12, 13, 14, or all 15 of induced PCOS phenotypic changes, symptoms, or outcomes listed in Table 5 compared to PCOS disease control subjects.

### Endometriosis Model:

An endometriosis mouse model is generated by inoculating endometrial fragments collected from a donor mouse into the peritoneal cavity of a recipient mouse. Endometriosis mice are treated with melatonin receptor agonist for a predetermined time (e.g. 10, 15, 20, 30 days) and the effect of treatment is measured by evaluating:
Lesion regression
Decrease in proMMP9 levels and/or expression and/or activity
Increase in TIMP1 levels and/or expression and/or activity and/or decreased proMMP9/TIMP1 ratio
Decrease in proMMP3 levels and/or expression and/or activity
Decrease in TNFα expression and/or levels
Increase in apoptosis and/or apoptosis markers (e.g. caspase3, caspase9)
reduction of pain and/or hyperalgesia and/or allodynia
decrease in inflammation and/or inflammation biomarkers.

Mice are allocated into Groups 1-6 (randomization by BW on Day 0). Groups 2-6 have disease induction. Group 1 is a vehicle control. Groups 1-6 receive the treatments according to Experimental Group listed in Table 6 on study design days of treatment.

TABLE 6
Endometriosis model experimental group and treatment dose

| Experimental Group | Treatment Dose |
|---|---|
| 1—Non disease control | N/A |
| 2—endometriosis Disease Control | N/A |
| 3—endometriosis + Melatonin analog QD low | 30 mg/kg body weight |
| 4—endometriosis + Melatonin analog QD high | 60 mg/kg body weight |
| 5—endometriosis + Melatonin analog BID | 30 mg/kg body weight |
| 6—endometriosis + Melatonin | 2 mg/kg body weight |

At end of study, remaining animals from Groups 1-6 are assayed for extent of endometriosis phenotypic changes and symptoms listed above in this example.

### Example 9: In Vivo Chemotherapy-Induced Ovarian Dysfunction Model

Melatonin has been shown to mitigate and protect metabolic and reproductive traits in chemotherapy tissue damage animal models. A compound functioning as a melatonin receptor agonist will cause one or more of the following phenotypic changes in CIOD rat/mouse models:
Attenuation of CIOD-induced primordial follicle loss
Decrease in growing follicle atresia
Decrease in CIOD-induced mitochondrial damage
Normalization of inflammatory biomarkers (C reactive protein (CRP), IL1 and TNF expression and plasma malondialdehyde (MDA) levels) and antioxidant biomarkers (total antioxidant capacity (TAC) levels and total glutathione (GSH) following chemotherapy administration
Reduction in extent of CIOD-induced ovarian apoptosis
Reduction in extent of CIOD-induced oxidative damage
Normalization of sex hormone levels
Improvement of follicular morphology
Normalization of granulosa cell proliferation.
Reduction of fertility loss following CIOD
Prevention of chemotherapy-induced ovarian reserve decline
Protection from chemotherapy-induced mitochondrial damage in the ovary
Restoration of ovarian mitochondrial function
Decrease in ovarian collagen deposition

A concurrent treatment CIOD model is made by dosing female rats with a single dose of cisplatin (7 mg/kg body weight; i.p. injection) on Day 1. Test animals additionally receive treatment of a melatonin receptor agonist (30 or 60 mg/kg body weight; via oral administration) once or twice daily for seven consecutive days starting on Day 1. Control animals do not receive treatment of a melatonin receptor agonist. All animals are maintained on a 12 hr light/12 hr dark cycle beginning at 6:00 AM. Following completion of all treatments, blood samples are taken on Day 8 from all animals and assayed for serum hormone levels, inflammatory biomarkers, and antioxidant biomarkers. Following collection of blood samples, animals are necropsied and ovarian cellular and tissue morphology is assayed by histological analysis to allow measurements of extent of tissue damage and CIOD phenotypes.

A pretreatment CIOD model is made. For test animals, female mice are pretreated with a melatonin receptor agonist (30 or 60 mg/kg body weight; via oral administration) once or twice daily for three days. On Day 3, all mice are administered a single dose of cyclophosphamide (200 mg/kg body weight; via i.p. injection). Test animals receive the cyclophosphamide dose 30 minutes after their final melatonin receptor agonist pretreatment. All animals are maintained on a 12 hr light/12 hr dark cycle beginning at 6:00 AM. On Day 10, blood samples are taken from all animals and assayed for serum hormone levels, inflammatory biomarkers, and antioxidant biomarkers. Following collection of blood samples, animals are necropsied and ovarian cellular and tissue morphology is assayed by histological analysis to allow measurements of extent of tissue damage and CIOD phenotypes.

Results will indicate that both concurrent treatment and pretreatment with a melatonin receptor agonist mitigate the effects of CIOD.

### Example 10: Patient Selection and Stratification and Biomarker Evaluation

There is a beneficial effect of melatonin on hyperandrogenism, inflammation, ART outcome, and metabolic function in PCOS patients or patients with metabolic disturbances. PCOS patients with these characteristics will be considered for treatment.

### Biochemical Endpoints:

Evaluation of melatonin receptor activation can be evaluated by looking at:
normalization of metabolic phenotype biomarkers, including HbA1c, fasting glucose, fasting insulin, homeostatic model assessment of insulin resistance (HOMO-IR), along with lipid profile measures
improvement of hyperandrogenism
normalization of inflammatory biomarkers (C reactive protein (CRP), IL1 and TNF expression and plasma malondialdehyde (MDA) levels) and antioxidant biomarkers (total antioxidant capacity (TAC) levels and total glutathione (GSH))
amelioration of ovulatory defects could be expected considering the positive effects of melatonin on ART outcomes and in improving ovarian function in vitro.

Results will indicate that PCOS patients selected having at least one of: hyperandrogenism, inflammation, unsuccessful ART outcome, or metabolic disturbances will benefit from undergoing a treatment comprising administering of a therapeutically effective amount of a melatonin receptor agonist according to a dosage regimen that continues at least until the patient demonstrates improvement in a biochemical endpoint. The improvement will include an improvement in at least one feature for which the patients were selected. Non-limiting examples of improvements include improvement of features of hyperandrogenism, normalization of one or more inflammatory biomarkers (e.g., C reactive protein (CRP), IL1 and TNF expression and plasma malondialdehyde (MDA) levels), normalization of one or more antioxidant biomarkers (e.g., total antioxidant capacity (TAC) levels and total glutathione (GSH)), improvement in ovulatory defects leading to improved chance of achieving successful ART outcomes, normalization of metabolic phenotype markers (e.g., HbA1c, fasting glucose, fasting insulin, homeostatic model assessment of insulin resistance (HOMO-IR), or normalization of lipid profile measure, or any combination thereof.

While the foregoing disclosure has been described in some detail for purposes of clarity and understanding, it will be clear to one skilled in the art from a reading of this disclosure that various changes in form and detail can be made without departing from the true scope of the disclosure. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

### ASPECTS

Some embodiments relate to any of the following aspects:
Aspect 1: A pharmaceutical composition comprising a therapeutically effective amount of a melatonin receptor agonist, wherein the pharmaceutical composition is formulated for:
   a. intravaginal;
   b. intrauterine; or
   c. systemic administration with reduced accumulation in central nervous system (CNS) compared to melatonin;
      wherein the therapeutically effective amount is effective to treat or ameliorate symptoms of polycystic ovary syndrome (PCOS), endometriosis, menopause, chemotherapy-induced ovarian dysfunction, or inflammation of a reproductive tract in a subject in need thereof.
Aspect 2: A pharmaceutical composition comprising a therapeutically effective amount of a melatonin receptor agonist, wherein the pharmaceutical composition is formulated for:
   a. intravaginal;
   b. intrauterine; or
   c. systemic administration with reduced accumulation in central nervous system (CNS) compared to melatonin;
wherein the therapeutically effective amount is effective to prevent, reduce, or inhibit one or more symptoms associated with chemotherapy-induced ovarian dysfunction, in a subject in need thereof.
Aspect 3: The pharmaceutical composition of aspect 1 or 2, wherein the melatonin receptor agonist comprises at least one of Circadin®, Slenyto®, Ramelteon, Tasimelteon, Agomelatine, TIK-301, Piromelatine, N-[2-(5-Chloro-2,6-dimethoxybenzoimidazol-1-yl)ethyl]acetamide (ACH000-143, (compound10b)), N-[3-(5-Chloro-2-ethoxy-6-methoxybenzoimidazol-1-yl) propyl]acetamide (compound 15a), N-[2-(2-methoxy-7,8-dihydro-6-oxa-1,3-diaza-as-indacen-1-yl)ethyl]acetamide (compound 19a), or a salt or derivative thereof, or a combination thereof.
Aspect 4: The pharmaceutical composition of aspect 1 or 2, wherein the melatonin receptor agonist comprises Circadin®.
Aspect 5: The pharmaceutical composition of aspect 1 or 2, wherein the melatonin receptor agonist comprises Slenyto®.
Aspect 6: The pharmaceutical composition of aspect 1 or 2, wherein the melatonin receptor agonist comprises Ramelteon.
Aspect 7: The pharmaceutical composition of aspect 1 or 2, wherein the melatonin receptor agonist comprises Tasimelteon.
Aspect 8: The pharmaceutical composition of aspect 1 or 2, wherein the melatonin receptor agonist comprises Agomelatine.
Aspect 9: The pharmaceutical composition of aspect 1 or 2, wherein the melatonin receptor agonist comprises TIK-301.
Aspect 10: The pharmaceutical composition of aspect 1 or 2, wherein the melatonin receptor agonist comprises Piromelatine.
Aspect 11: The pharmaceutical composition of aspect 1 or 2, wherein the melatonin receptor agonist comprises N-[2-(5-Chloro-2,6-dimethoxybenzoimidazol-1-yl)ethyl]acetamide.
Aspect 12: The pharmaceutical composition of aspect 1 or 2, wherein the melatonin receptor agonist comprises N-[3-(5-Chloro-2-ethoxy-6-methoxybenzoimidazol-1-yl) propyl]acetamide.
Aspect 13: The pharmaceutical composition of aspect 1 or 2, wherein the melatonin receptor agonist comprises N-[2-(2-methoxy-7,8-dihydro-6-oxa-1,3-diaza-as-indacen-1-yl)ethyl]acetamide.
Aspect 14: The pharmaceutical composition of any one of aspects 1-13, wherein the melatonin receptor agonist non-selectively activates type 1A (MT₁) and type 1B (MT₂) melatonin receptors.
Aspect 15: The pharmaceutical composition of any one of aspects 1-13, wherein the melatonin receptor agonist preferentially activates type 1A (MT₁) melatonin receptors.
Aspect 16: The pharmaceutical composition of any one of aspects 1-13, wherein the melatonin receptor agonist preferentially activates type 1B (MT₂) melatonin receptors.
Aspect 17: The pharmaceutical composition of any one of aspects 1-16, wherein the pharmaceutical composition is formulated as a gel, an ointment, a solution, a powder, a paste, a foam, a cream or a lotion.
Aspect 18: The pharmaceutical composition of any one of aspects 1-16, wherein the pharmaceutical composition is formulated to be delivered via a drug delivery device.
Aspect 19: The pharmaceutical composition of aspect 18, wherein the drug delivery device is a vaginal ring, a vaginal tablet, a pessary, a suppository, a patch, or an intrauterine device.
Aspect 20: The pharmaceutical composition of aspect 18 or 19, wherein the drug delivery device is configured to be placed in proximity to a reproductive organ of the subject.
Aspect 21: The pharmaceutical composition of aspect 19 or 20, wherein the drug delivery device is the intrauterine device and configured to be placed in proximity to a reproductive organ of the subject.
Aspect 22: The pharmaceutical composition of aspect 21, wherein the intrauterine device is configured for time-controlled release or remote-controlled release of one or a plurality of doses of the melatonin receptor agonist.
Aspect 23: The pharmaceutical composition of any one of aspects 1-22, wherein the pharmaceutical composition is formulated for delayed release, sustained release, extended release, prolonged release, or slow release.
Aspect 24: The pharmaceutical composition of any one of aspects 1-23, wherein the pharmaceutical composition comprises the melatonin receptor agonist at an amount to be effective at least about 1 week, 2 weeks, 4 weeks, 2 months, or 6 months.
Aspect 25: The pharmaceutical composition of any one of aspects 1-24, wherein less than about 50%, 40%, 30%, 20%, 15%, 10% or 5% of the pharmaceutical composition accumulates in the CNS of the subject.
Aspect 26: The pharmaceutical composition of any one of aspects 4 or 14-24, wherein less than about 50%, 40%, 30%, 20%, 15%, 10% or 5% of Circadin® accumulates in the CNS of the subject.
Aspect 27: The pharmaceutical composition of any one of aspects 5 or 14-24, wherein less than about 50%, 40%, 30%, 20%, 15%, 10% or 5% of Slenyto® accumulates in the CNS of the subject.
Aspect 28: The pharmaceutical composition of any one of aspects 6 or 14-24, wherein less than about 50%, 40%, 30%, 20%, 15%, 10% or 5% of Ramelteon accumulates in the CNS of the subject.
Aspect 29: The pharmaceutical composition of any one of aspects 7 or 14-24, wherein less than about 50%, 40%, 30%, 20%, 15%, 10% or 5% of Tasimelteon accumulates in the CNS of the subject.
Aspect 30: The pharmaceutical composition of any one of aspects 8 or 14-24, wherein less than about 50%, 40%, 30%, 20%, 15%, 10% or 5% of Agomelatine accumulates in the CNS of the subject.
Aspect 31: The pharmaceutical composition of any one of aspects 9 or 14-24, wherein less than about 50%, 40%, 30%, 20%, 15%, 10% or 5% of TIK-301 accumulates in the CNS of the subject.
Aspect 32: The pharmaceutical composition of any one of aspects 10 or 14-24, wherein less than about 50%, 40%, 30%, 20%, 15%, 10% or 5% of Piromelatine accumulates in the CNS of the subject.
Aspect 33: The pharmaceutical composition of any one of aspects 11 or 14-24, wherein less than about 50%, 40%, 30%, 20%, 15%, 10% or 5% of N-[2-(5-Chloro-2,6-dimethoxybenzoimidazol-1-yl)ethyl]acetamide accumulates in the CNS of the subject.
Aspect 34: The pharmaceutical composition of any one of aspects 12 or 14-24, wherein less than about 50%, 40%, 30%, 20%, 15%, 10% or 5% of N-[3-(5-Chloro-2-ethoxy-6-methoxybenzoimidazol-1-yl) propyl]acetamide accumulates in the CNS of the subject.
Aspect 35: The pharmaceutical composition of any one of aspects 13-24, wherein less than about 50%, 40%, 30%, 20%, 15%, 10% or 5% of N-[2-(2-methoxy-7,8-dihydro-6-oxa-1,3-diaza-as-indacen-1-yl)ethyl]acetamide accumulates in the CNS of the subject.
Aspect 36: The pharmaceutical composition of any one of aspects 1-35, wherein the melatonin receptor agonist has a reduced rate of passive diffusion compared to melatonin.
Aspect 37: The pharmaceutical composition of any one of aspects 1-36, wherein the melatonin receptor agonist has an increased rate of cerebrospinal fluid (CSF) expulsion via blood-brain barrier (BBB) egress transporters compared to melatonin.
Aspect 38: The pharmaceutical composition of any one of aspects 1-37, wherein the melatonin receptor agonist has an increased rate of CNS metabolism compared to melatonin.
Aspect 39: The pharmaceutical composition of any one of aspects 1-38, wherein the melatonin receptor agonist has an increased rate of CNS clearance compared to melatonin.
Aspect 40: The pharmaceutical composition of any one of aspects 1-39, wherein the melatonin receptor agonist has increased binding to one or a plurality of CNS proteins compared to melatonin.
Aspect 41: The pharmaceutical composition of any one of aspects 1-40, wherein the melatonin receptor agonist has increased binding to one or a plurality of peripheral proteins compared to melatonin.
Aspect 42: The pharmaceutical composition of any one of aspects 1-41, wherein the melatonin receptor agonist is conjugated or encapsulated with a molecule that has a low capacity to cross the blood brain barrier.
Aspect 43: The pharmaceutical composition of aspect 42, wherein the molecule is a hydrophilic molecule, a carbohydrate molecule, a peptide, or a synthetic molecule.
Aspect 44: The pharmaceutical composition of any one of aspects 1 or 3-43, for use in the treatment of polycystic ovary syndrome (PCOS) in a subject in need thereof.
Aspect 45: The pharmaceutical composition of any one of aspects 1 or 3-43, for use in the treatment of endometriosis in a subject in need thereof.
Aspect 46: The pharmaceutical composition of any one of aspects 1 or 3-43, for use in the treatment of symptoms of menopause in a subject in need thereof.
Aspect 47: The pharmaceutical composition of any one of aspects 1-43, for use in the treatment of symptoms of chemotherapy-induced ovarian dysfunction in a subject in need thereof.
Aspect 48: The pharmaceutical composition of any one of aspects 2-43, for use in the prevention of symptoms of chemotherapy-induced ovarian dysfunction in a subject in need thereof.
Aspect 49: The pharmaceutical composition of any one of aspects 2-43, for use in the reduction of symptoms of chemotherapy-induced ovarian dysfunction in a subject in need thereof.
Aspect 50: The pharmaceutical composition of any one of aspects 2-43, for use in the inhibition of symptoms of chemotherapy-induced ovarian dysfunction in a subject in need thereof.
Aspect 51: The pharmaceutical composition of any one of aspects 1-43, for use in the treatment of inflammation of a reproductive tract in a subject in need thereof.
Aspect 52: The pharmaceutical composition of any one of aspects 1-51, wherein the therapeutically effective amount when administered to the subject is effective to prevent, reduce, or inhibit one or more symptoms associated with polycystic ovary syndrome (PCOS), endometriosis, menopause, chemotherapy-induced ovarian dysfunction, or inflammation of a reproductive tract in the subject.
Aspect 53: A pharmaceutical composition comprising a therapeutically effective amount of a melatonin receptor agonist, wherein the pharmaceutical composition is formulated for:
a. intravaginal;
b. intrauterine; or
c. systemic administration with reduced accumulation in central nervous system (CNS) compared to melatonin;
wherein the therapeutically effective amount is effective to prevent, reduce, or inhibit
one or more symptoms associated with chemotherapy-induced ovarian dysfunction, in a subject in need thereof.
Aspect 54: A method of treating or ameliorating a symptom of polycystic ovary syndrome (PCOS), endometriosis, menopause, chemotherapy-induced ovarian dysfunction, or inflammation of a reproductive tract in a subject in need thereof, comprising:
administering the pharmaceutical composition of any one of aspects 1-52 to a subject in need thereof.
Aspect 55: The method of aspect 54, wherein the pharmaceutical composition is administered intravaginally, intrauterine or systemically with reduced accumulation in the CNS of the subject compared to melatonin.
Aspect 56: The method of aspect 54 or 55, wherein the pharmaceutical composition is administered intravaginally and comprises administering the pharmaceutical composition to a dermal or a transmucosal layer of a vaginal wall of the subject.
Aspect 57: The method of any one of aspects 54-56, wherein the symptom comprises an abdominal pain, a back pain, a chronic pelvic pain, a dysmenorrhea, an amenorrhea, an oligomenorrhea, glucose intolerance, insulin resistance, hyperandrogenemia, liver steatosis, hirsutism, infertility, weight gain, bone density loss, hair loss, hypertension, or a combination thereof.
Aspect 58: The method of any one of aspects 54-57, wherein the symptom comprises hot flashes, osteoporosis, risk of infertility, infertility, sleep disturbances, joint pain, anxiety, depression, sexual dysfunction, increased risk of cardiovascular disease, loss of primordial ovarian follicles, accelerated activation of primordial ovarian follicles, follicular atresia, stromal tissue damage, ovarian vascular damage, ovarian inflammation, reduction in ovarian reserve, increase in rate of age-dependent loss of ovarian reserve, induction of fibrosis, early menopause, premature ovarian insufficiency, premature ovarian failure, induction of apoptosis in mature ovarian follicles, decreased estrogen production, decreased anti-müllerian hormone (AMH) production, follicular stimulating hormone (FSH) levels, decreased plasma levels of estradiol, or any combination thereof.
Aspect 59: The method of any one of aspects 54-58, wherein the administering comprises administration according to a dose and a schedule effective to reduce abdominal pain, back pain, chronic pelvic pain, dysmenorrhea, an amenorrhea, an oligomenorrhea, glucose intolerance, insulin resistance, hyperandrogenemia, liver steatosis, hirsutism, infertility, weight gain, bone density loss, hair loss, hypertension or a combination thereof at least 20%, at least 30%, at least 40% within 24 hours, 48 hours, or 72 hours measured by a numerical rating scales (NRS).
Aspect 60: The method of any one of aspects 54-59, wherein the administering comprises administration at least about every day, every 2 days, every 3 days, every 7 days, every 14 days, every 28 days, every 2 months, or every 6 months.
Aspect 61: The method of any one of aspects 54-60, wherein the administering comprises administration continuously for a duration of treatment.
Aspect 62: The method of any one of aspects 54-61, wherein the administering is initiated about 15 days, 14 days, 13 days, 12 days, 11 days, 10 days, 9 days, 8 days, 7 days, 6 days, 5 days, 4 days, 3 days, 2 days, 1 day, 18 hours, 12 hours, 8 hours, 6 hours, 4 hours, 2 hours, or 1 hour prior to initiation of a chemotherapy treatment in the subject.
Aspect 63: The method of any one of aspects 54-62, wherein the administering comprises administration at certain times of day or night according to a circadian rhythm of the subject.
Aspect 64: The method of any one of aspects 54-63, wherein the pharmaceutical composition regulates activity of melatonin receptors outside of the CNS according to the circadian rhythm of the subject.
Aspect 65: The method of any one of aspects 54-64, wherein the administering comprises administration at certain times of day or night irrespective of circadian fluctuations in plasma melatonin levels of the subject.
Aspect 66: The method of any one of aspects 54-65, wherein the administering does not disrupt a circadian rhythm of plasma melatonin levels of the subject.
Aspect 67: The method of any one of aspects 54-66, wherein the administering comprises administration at any time during a 24 hour period.
Aspect 68: The method of any one of aspects 54-67, wherein the administering has a reduced effect on a melatonin receptor mediated CNS behavior compared to administering an equivalent amount of melatonin.
Aspect 69: The method of any one of aspects 54-68, wherein the administering has a reduced effect on a melatonin receptor mediated CNS behavior during nighttime hours compared to administering an equivalent amount of melatonin.
Aspect 70: The method of aspect 68 or 69, wherein the melatonin receptor mediated CNS behavior comprises circadian rhythm, addiction, sleep behavior, motor control, or memory extinction.
Aspect 71: Use of the pharmaceutical composition of any one of aspects 1-52, for manufacture of a medicament for treating polycystic ovary syndrome (PCOS) in a subject in need thereof.
Aspect 72: Use of the pharmaceutical composition of any one of aspects 1-52, for manufacture of a medicament for treating endometriosis in a subject in need thereof.
Aspect 73: Use of the pharmaceutical composition of any one of aspects 1-52, for manufacture of a medicament for treating symptoms of menopause in a subject in need thereof
Aspect 74: Use of the pharmaceutical composition of any one of aspects 1-53, for manufacture of a medicament for treating symptoms of chemotherapy-induced ovarian dysfunction in a subject in need thereof.
Aspect 75: Use of the pharmaceutical composition of any one of aspects 1-53, for manufacture of a medicament for treating symptoms of chemotherapy-induced ovarian failure in a subject in need thereof.
Aspect 76: Use of the pharmaceutical composition of any one of aspects 1-53, for manufacture of a medicament for treating inflammation of a reproductive tract in a subject in need thereof.
Aspect 77: Use of any one of aspects 70-75, wherein the medicament is prepared to be administered in a dosage regime comprising administering at least about every day, every 2 days, every 3 days, every 7 days, every 14 days, every 28 days, every 2 months, or every 6 months.
Aspect 78: Use of any one of aspects 71-77, wherein the medicament is prepared to be administered in a dosage regime comprising administering continuously for a duration of treatment.
Aspect 79: Use of any one of aspects 71-78, the medicament is prepared to be administered in a dosage regime comprising initiation of administering about 15 days, 14 days, 13 days, 12 days, 11 days, 10 days, 9 days, 8 days, 7 days, 6 days, 5 days, 4 days, 3 days, 2 days, 1 day, 18 hours, 12 hours, 8 hours, 6 hours, 4 hours, 2 hours, or 1 hour prior to initiation of a chemotherapy treatment in the subject.
Aspect 80: Use of any one of aspects 71-79, wherein the medicament is prepared to be administered in a dosage regime comprising administering at certain times of day or night according to a circadian rhythm of the subject.
Aspect 81: Use of any one of aspects 71-79, wherein the medicament is prepared to be administered in a dosage regime comprising administering at certain times of day or night irrespective of circadian fluctuations in plasma melatonin levels.
Aspect 82: Use of any one of aspects 71-81, wherein the medicament is prepared to be administered in a dosage regime comprising administering that does not disrupt a circadian rhythm of plasma melatonin levels.
Aspect 83: Use of any one of aspects 71-82, wherein the medicament is prepared to be administered in a dosage regime comprising administering at any time during a 24 hour period.
Aspect 84: Use of any one of aspects 71-83, wherein the medicament is prepared to be administered in a dosage regime comprising administering having a reduced effect on a melatonin receptor mediated CNS behavior compared to administering an equivalent amount of melatonin.
Aspect 85: Use of any one of aspects 70-84, wherein the medicament is prepared to be administered in a dosage regime comprising administering having a reduced effect on a melatonin receptor mediated CNS behavior during nighttime hours compared to administering an equivalent amount of melatonin.
Aspect 86: A kit comprising the pharmaceutical composition of any one of aspects 1-52, and instructions for use.
Aspect 87: The kit of aspect 86, wherein the instructions for use designate one or more indications in a subject in need of treatment, wherein the one or more indications comprise polycystic ovary syndrome (PCOS), endometriosis, menopause, chemotherapy-induced ovarian dysfunction (CIOD), or inflammation of a reproductive tract, or a combination thereof.
Aspect 88: The kit of aspect 86 or 87, further comprising a drug delivery device for administering the pharmaceutical composition to a subject.
Aspect 89: The kit of any one of aspects 86-88, wherein the drug delivery device is a vaginal ring, a vaginal tablet, a pessary, a suppository, a patch, or an intrauterine device.
Aspect 90: The kit of any one of aspects 86-89, wherein the drug delivery device is configured to be placed in proximity to a reproductive organ of the subject.
Aspect 91: The kit of any one of aspects 86-90, wherein the drug delivery device is the intrauterine device and configured to be placed in proximity to a reproductive organ of the subject.
Aspect 92: The kit of any one of aspects 86-91, wherein the intrauterine device is configured for time-controlled release or remote-controlled release of one or a plurality of doses of the pharmaceutical composition.

What is claimed is:

1. A pharmaceutical composition comprising a therapeutically effective amount of a melatonin receptor agonist, wherein the pharmaceutical composition is formulated for:
a. intravaginal;
b. intrauterine; or
c. systemic administration with reduced accumulation in central nervous system (CNS) compared to melatonin;
wherein the therapeutically effective amount is effective to treat or ameliorate symptoms of polycystic ovary syndrome (PCOS), endometriosis, menopause, chemotherapy-induced ovarian dysfunction, or inflammation of a reproductive tract in a subject in need thereof.

2. The pharmaceutical composition of claim 1, wherein the melatonin receptor agonist comprises at least one of Circadin®, Slenyto®, Ramelteon, Tasimelteon, Agomelatine, TIK-301, Piromelatine, N-[2-(5-Chloro-2,6-dimethoxybenzoimidazol-1-yl)ethyl]acetamide (ACH000-143, (compound10b)), N-[3-(5-Chloro-2-ethoxy-6-methoxybenzoimidazol-1-yl) propyl]acetamide (compound 15a), N-[2-(2-methoxy-7,8-dihydro-6-oxa-1,3-diaza-as-indacen-1-yl)ethyl]acetamide (compound 19a), or a salt or derivative thereof, or a combination thereof.

3. The pharmaceutical composition of claim 1, wherein the melatonin receptor agonist comprises Circadin®.

4. The pharmaceutical composition of claim 1, wherein the melatonin receptor agonist comprises Slenyto®.

5. The pharmaceutical composition of claim 1, wherein the melatonin receptor agonist comprises Ramelteon.

6. The pharmaceutical composition of claim 1, wherein the melatonin receptor agonist comprises Tasimelteon.

7. The pharmaceutical composition of claim 1, wherein the melatonin receptor agonist comprises Agomelatine.

8. The pharmaceutical composition of claim 1, wherein the melatonin receptor agonist comprises TIK-301.

9. The pharmaceutical composition of claim 1, wherein the melatonin receptor agonist comprises Piromelatine.

10. The pharmaceutical composition of claim 1, wherein the melatonin receptor agonist comprises N-[2-(5-Chloro-2,6-dimethoxybenzoimidazol-1-yl)ethyl]acetamide.

11. The pharmaceutical composition of claim 1, wherein the melatonin receptor agonist comprises N-[3-(5-Chloro-2-ethoxy-6-methoxybenzoimidazol-1-yl) propyl]acetamide.

12. The pharmaceutical composition of claim 1, wherein the melatonin receptor agonist comprises N-[2-(2-methoxy-7,8-dihydro-6-oxa-1,3-diaza-as-indacen-1-yl)ethyl]acetamide.

13. The pharmaceutical composition of claim 1, wherein the melatonin receptor agonist non-selectively activates type 1A (MT₁) and type 1B (MT₂) melatonin receptors.

14. The pharmaceutical composition of claim 1, wherein the melatonin receptor agonist preferentially activates type 1A (MT₁) melatonin receptors.

15. The pharmaceutical composition of claim 1, wherein the melatonin receptor agonist preferentially activates type 1B (MT₂) melatonin receptors.

16. The pharmaceutical composition of claim 1, wherein the pharmaceutical composition is formulated as a gel, an ointment, a solution, a powder, a paste, a foam, a cream or a lotion.

17. The pharmaceutical composition of claim 1, wherein the pharmaceutical composition is formulated to be delivered via a drug delivery device.

18. The pharmaceutical composition of claim 17, wherein the drug delivery device is a vaginal ring, a vaginal tablet, a pessary, a suppository, a patch, or an intrauterine device.

19. The pharmaceutical composition of claim 18, wherein the drug delivery device is configured to be placed in proximity to a reproductive organ of the subject.

20. The pharmaceutical composition of claim 18, wherein the drug delivery device is the intrauterine device and configured to be placed in proximity to a reproductive organ of the subject.

21. The pharmaceutical composition of claim 20, wherein the intrauterine device is configured for time-controlled release or remote-controlled release of one or a plurality of doses of the melatonin receptor agonist.

22. The pharmaceutical composition of claim 17, wherein the pharmaceutical composition is formulated for delayed release, sustained release, extended release, prolonged release, or slow release.

23. The pharmaceutical composition of claim 1, wherein the pharmaceutical composition comprises the melatonin receptor agonist at an amount to be effective at least about 1 week, 2 weeks, 4 weeks, 2 months, or 6 months.

24. The pharmaceutical composition of claim 1, wherein less than about 50%, 40%, 30%, 20%, 15%, 10% or 5% of the pharmaceutical composition accumulates in the CNS of the subject.

25. The pharmaceutical composition of claim 3, wherein less than about 50%, 40%, 30%, 20%, 15%, 10% or 5% of Circadin® accumulates in the CNS of the subject.

26. The pharmaceutical composition of claim 4, wherein less than about 50%, 40%, 30%, 20%, 15%, 10% or 5% of Slenyto® accumulates in the CNS of the subject.

27. The pharmaceutical composition of claim 5, wherein less than about 50%, 40%, 30%, 20%, 15%, 10% or 5% of Ramelteon accumulates in the CNS of the subject.

28. The pharmaceutical composition of claim 6, wherein less than about 50%, 40%, 30%, 20%, 15%, 10% or 5% of Tasimelteon accumulates in the CNS of the subject.

29. The pharmaceutical composition of claim 7, wherein less than about 50%, 40%, 30%, 20%, 15%, 10% or 5% of Agomelatine accumulates in the CNS of the subject.

30. The pharmaceutical composition of claim 8, wherein less than about 50%, 40%, 30%, 20%, 15%, 10% or 5% of TIK-301 accumulates in the CNS of the subject.

31. The pharmaceutical composition of claim 9, wherein less than about 50%, 40%, 30%, 20%, 15%, 10% or 5% of Piromelatine accumulates in the CNS of the subject.

32. The pharmaceutical composition of claim 10, wherein less than about 50%, 40%, 30%, 20%, 15%, 10% or 5% of N-[2-(5-Chloro-2,6-dimethoxybenzoimidazol-1-yl)ethyl]acetamide accumulates in the CNS of the subject.

33. The pharmaceutical composition of claim 11, wherein less than about 50%, 40%, 30%, 20%, 15%, 10% or 5% of N-[3-(5-Chloro-2-ethoxy-6-methoxybenzoimidazol-1-yl) propyl]acetamide accumulates in the CNS of the subject.

34. The pharmaceutical composition of claim 12, wherein less than about 50%, 40%, 30%, 20%, 15%, 10% or 5% of N-[2-(2-methoxy-7,8-dihydro-6-oxa-1,3-diaza-as-indacen-1-yl)ethyl]acetamide accumulates in the CNS of the subject.

35. The pharmaceutical composition of claim 1, wherein the melatonin receptor agonist has a reduced rate of passive diffusion compared to melatonin.

36. The pharmaceutical composition of claim 1, wherein the melatonin receptor agonist has an increased rate of cerebrospinal fluid (CSF) expulsion via blood-brain barrier (BBB) egress transporters compared to melatonin.

37. The pharmaceutical composition of claim 1, wherein the melatonin receptor agonist has an increased rate of CNS metabolism compared to melatonin.

38. The pharmaceutical composition of claim 1, wherein the melatonin receptor agonist has an increased rate of CNS clearance compared to melatonin.

39. The pharmaceutical composition of claim 1, wherein the melatonin receptor agonist has increased binding to one or a plurality of CNS proteins compared to melatonin.

40. The pharmaceutical composition of claim 1, wherein the melatonin receptor agonist has increased binding to one or a plurality of peripheral proteins compared to melatonin.

41. The pharmaceutical composition of claim 1, wherein the melatonin receptor agonist is conjugated or encapsulated with a molecule that has a low capacity to cross the blood brain barrier.

42. The pharmaceutical composition of claim 41, wherein the molecule is a hydrophilic molecule, a carbohydrate molecule, a peptide, or a synthetic molecule.

43. The pharmaceutical composition of claim 1, for use in the treatment of polycystic ovary syndrome (PCOS) in a subject in need thereof.

44. The pharmaceutical composition of claim 1, for use in the treatment of endometriosis in a subject in need thereof.

45. The pharmaceutical composition of claim 1, for use in the treatment of symptoms of menopause in a subject in need thereof.

46. The pharmaceutical composition of claim 1, for use in the treatment of symptoms of chemotherapy-induced ovarian dysfunction (CIOD) in a subject in need thereof.

47. The pharmaceutical composition of claim 1, for use in the treatment of inflammation of a reproductive tract in a subject in need thereof.

48. The pharmaceutical composition of claim 1, for use in the treatment of symptoms of chemotherapy-induced ovarian failure (CIOF) in a subject in need thereof.

49. The pharmaceutical composition of claim 1, for use in the prevention of symptoms of chemotherapy-induced ovarian dysfunction in a subject in need thereof.

50. The pharmaceutical composition of claim 1, for use in the reduction of symptoms of chemotherapy-induced ovarian dysfunction in a subject in need thereof.

51. The pharmaceutical composition of claim 1, for use in the inhibition of symptoms of chemotherapy-induced ovarian dysfunction in a subject in need thereof.

52. The pharmaceutical composition of any one of claims 1-51, wherein the therapeutically effective amount when administered to the subject is effective to prevent, reduce, or inhibit one or more symptoms associated with polycystic ovary syndrome (PCOS), endometriosis, menopause, chemotherapy-induced ovarian dysfunction, or inflammation of a reproductive tract in the subject.

53. A method of treating or ameliorating a symptom of polycystic ovary syndrome (PCOS), endometriosis, menopause, chemotherapy-induced ovarian dysfunction, or inflammation of a reproductive tract in a subject in need thereof, comprising:
administering the pharmaceutical composition of any one of claims 1-52 to a subject in need thereof.

54. The method of claim 53, wherein the pharmaceutical composition is administered intravaginally, intrauterine or systemically with reduced accumulation in the CNS of the subject compared to melatonin.

55. The method of claim 54, wherein the pharmaceutical composition is administered intravaginally and comprises administering the pharmaceutical composition to a dermal or a transmucosal layer of a vaginal wall of the subject.

56. The method of any one of claims 53-55, wherein the symptom comprises an abdominal pain, a back pain, a chronic pelvic pain, a dysmenorrhea, an amenorrhea, an oligomenorrhea, glucose intolerance, insulin resistance, hyperandrogenemia, liver steatosis, hirsutism, infertility, weight gain, bone density loss, hair loss, hypertension or a combination thereof.

57. The method of any one of claims 53-56, wherein the administering comprises administration according to a dose and a schedule effective to reduce abdominal pain, back pain, chronic pelvic pain, dysmenorrhea, an amenorrhea, an oligomenorrhea, glucose intolerance, insulin resistance, hyperandrogenemia, liver steatosis, hirsutism, infertility, weight gain, bone density loss, hair loss, hypertension or a combination thereof at least 20%, at least 30%, at least 40% within 24 hours, 48 hours, or 72 hours measured by a numerical rating scales (NRS).

58. The method of any one of claims 53-57, wherein the administering comprises administration at least about every day, every 2 days, every 3 days, every 7 days, every 14 days, every 28 days, every 2 months, or every 6 months.

59. The method of any one of claims 53-58, wherein the administering comprises administration continuously for a duration of treatment.

60. The method of any one of claims 53-59, wherein the administering comprises administration at certain times of day or night according to a circadian rhythm of the subject.

61. The method of claim 60, wherein the pharmaceutical composition regulates activity of melatonin receptors outside of the CNS according to the circadian rhythm of the subject.

62. The method of any one of claims 53-61, wherein the administering comprises administration at certain times of day or night irrespective of circadian fluctuations in plasma melatonin levels of the subject.

63. The method of any one of claims 53-62, wherein the administering does not disrupt a circadian rhythm of plasma melatonin levels of the subject.

64. The method of any one of claims 53-63, wherein the administering comprises administration at any time during a 24 hour period.

65. The method of any one of claims 53-64, wherein the administering has a reduced effect on a melatonin receptor mediated CNS behavior compared to administering an equivalent amount of melatonin.

66. The method of any one of claims 53-65, wherein the administering has a reduced effect on a melatonin receptor mediated CNS behavior during nighttime hours compared to administering an equivalent amount of melatonin.

67. The method of claim 65 or 66, wherein the melatonin receptor mediated CNS behavior comprises circadian rhythm, addiction, sleep behavior, motor control, or memory extinction.

68. Use of the pharmaceutical composition of any one of claims 1-52, for manufacture of a medicament for treating polycystic ovary syndrome (PCOS) in a subject in need thereof.

69. Use of the pharmaceutical composition of any one of claims 1-52, for manufacture of a medicament for treating endometriosis in a subject in need thereof.

70. Use of the pharmaceutical composition of any one of claims 1-52, for manufacture of a medicament for treating symptoms of menopause in a subject in need thereof.

71. Use of the pharmaceutical composition of any one of claims 1-52, for manufacture of a medicament for treating inflammation of a reproductive tract in a subject in need thereof.

72. Use of the pharmaceutical composition of any one of claims 1-52, for manufacture of a medicament for treating chemotherapy-induced ovarian dysfunction (CIOD) in a subject in need thereof.

73. Use of any one of claims 68-72, wherein the medicament is prepared to be administered in a dosage regime comprising administering at least about every day, every 2 days, every 3 days, every 7 days, every 14 days, every 28 days, every 2 months, or every 6 months.

74. Use of any one of claims 68-73, wherein the medicament is prepared to be administered in a dosage regime comprising pretreatment that begins about 15 days, 14 days, 13 days, 12 days, 11 days, 10 days, 9 days, 8 days, 7 days, 6 days, 5 days, 4 days, 3 days, 2 days, 1 day, 18 hours, 12 hours, 8 hours, 6 hours, 4 hours, 2 hours, or 1 hour prior to initiation of a chemotherapy treatment in the subject.

75. Use of any one of claims 68-74, wherein the medicament is prepared to be administered in a dosage regime comprising administering continuously for a duration of treatment.

76. Use of any one of claims 68-75, wherein the medicament is prepared to be administered in a dosage regime comprising administering at certain times of day or night according to a circadian rhythm of the subject.

77. Use of any one of claims 68-76, wherein the medicament is prepared to be administered in a dosage regime comprising administering at certain times of day or night irrespective of circadian fluctuations in plasma melatonin levels.

78. Use of any one of claims 68-77, wherein the medicament is prepared to be administered in a dosage regime comprising administering that does not disrupt a circadian rhythm of plasma melatonin levels.

79. Use of any one of claims 68-78, wherein the medicament is prepared to be administered in a dosage regime comprising administering at any time during a 24 hour period.

80. Use of any one of claims 68-79, wherein the medicament is prepared to be administered in a dosage regime comprising administering having a reduced effect on a melatonin receptor mediated CNS behavior compared to administering an equivalent amount of melatonin.

81. Use of any one of claims 68-80, wherein the medicament is prepared to be administered in a dosage regime comprising administering having a reduced effect on a melatonin receptor mediated CNS behavior during nighttime hours compared to administering an equivalent amount of melatonin.

82. A kit comprising the pharmaceutical composition of any one of claims 1-52, and instructions for use.

83. The kit of claim 82, wherein the instructions for use designate one or more indications in a subject in need of treatment, wherein the one or more indications comprise polycystic ovary syndrome (PCOS), endometriosis, menopause, chemotherapy-induced ovarian dysfunction (CIOD), chemotherapy-induced ovarian failure (CIOF), or inflammation of a reproductive tract, or a combination thereof.

84. The kit of claim 82 or 83, further comprising a drug delivery device for administering the pharmaceutical composition to a subject.

85. The kit of claim 84, wherein the drug delivery device is a vaginal ring, a vaginal tablet, a pessary, a suppository, a patch, or an intrauterine device.

86. The kit of claim 84 or 85, wherein the drug delivery device is configured to be placed in proximity to a reproductive organ of the subject.

87. The kit of any one of claims 84-86, wherein the drug delivery device is the intrauterine device and configured to be placed in proximity to a reproductive organ of the subject.

88. The kit of any one of claims 84-87, wherein the intrauterine device is configured for time-controlled release or remote-controlled release of one or a plurality of doses of the pharmaceutical composition formulated for delayed release, sustained release, extended release, prolonged release, or slow release.
